SEQUENCE LISTING (103 sequences)

SEQ ID NO: 1 - protein, 417 aa, Escherichia coli [sequence omitted]

SEQ ID NO: 2 - DNA, 1254 bp, Escherichia coli
  misc_feature 1..1254: note = Nucleic acid sequence encoding LacY, GenBank ID: NC_000913.3
  source 1..1254: mol_type = genomic DNA
atgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcgggctgcgcaaatacctgctgtggattattaccggcatgttagtgatgtttgcgccgttctttatttttatcttcgggccactgttacaatacaacattttagtaggatcgattgttggtggtatttatctaggcttttgttttaacgccggtgcgccagcagtagaggcatttattgagaaagtcagccgtcgcagtaatttcgaatttggtcgcgcgcggatgtttggctgtgttggctgggcgctgtgtgcctcgattgtcggcatcatgttcaccatcaataatcagtttgttttctggctgggctctggctgtgcactcatcctcgccgttttactctttttcgccaaaacggatgcgccctcttctgccacggttgccaatgcggtaggtgccaaccattcggcatttagccttaagctggcactggaactgttcagacagccaaaactgtggtttttgtcactgtatgttattggcgtttcctgcacctacgatgtttttgaccaacagtttgctaatttctttacttcgttctttgctaccggtgaacagggtacgcgggtatttggctacgtaacgacaatgggcgaattacttaacgcctcgattatgttctttgcgccactgatcattaatcgcatcggtgggaaaaacgccctgctgctggctggcactattatgtctgtacgtattattggctcatcgttcgccacctcagcgctggaagtggttattctgaaaacgctgcatatgtttgaagtaccgttcctgctggtgggctgctttaaatatattaccagccagtttgaagtgcgtttttcagcgacgatttatctggtctgtttctgcttctttaagcaactggcgatgatttttatgtctgtactggcgggcaatatgtatgaaagcatcggtttccagggcgcttatctggtgctgggtctggtggcgctgggcttcaccttaatttccgtgttcacgcttagcggccccggcccgctttccctgctgcgtcgtcaggtgaatgaagtcgcttaa

SEQ ID NO: 3 - protein, 433 aa, Escherichia coli
  REGION 1..433: note = MISC_FEATURE - Lactose permease, GenBank ID: WP_042094275.1
MYYLKNTNFWMFGLFFFFYFFIMGAYFPFFPIWLHDINHISKSDTGIIFAAISLFSLLFQPLFGLLSDKLGLRKYLLWIITGMLVMFAPFFIFIFGPLLQYNILVGSIVGGIYLGFCFNAGAPAVEAFIEKVSRRSNFEFGRARMFGCVGWALCASIVGIMFTINNQFVFWLGSGCALILAVLLFFAKTDAPSSATVANAVGANHSAFSLKLALELFRQPKLWFLSLYVIGVSCTYDVFDQQFANFFTSFFATGEQGTRVFGYVTTMGELLNASIMFFAPLIINRIGGKNALLLAGTIMSVRIIGSSFATSALEVVILKTLHMFEVPFLLVGCFKYITSQFEVRFSATIYLVCFCFFKQLAMIFMSVLAGNMYESIGFQGAYLVLGLVALGFTLISVFTLSGPGPLSLLRRQVNEVAYGCGASALSDQHIRAE

SEQ ID NO: 4 - protein, 417 aa, Escherichia coli [sequence omitted]

SEQ ID NO: 5 - protein, 417 aa, Campylobacter jejuni
  REGION 1..417: note = MISC_FEATURE - Lactose permease GenBank ID: WP_089607162.1
MYYLKNTNFWMFGLFFFFYFFIMGAYFPFFPIWLHDINHISKSDTGIIFAAISLFSLLFQPLFGLLSDKLGLRKYLLWIITGMLVMFAPFFIFIFGPLLQYNILVGSIVGGIYLGFCFNAGAPAVEAFIEKVSRRSNFEFGRARMFGCVGWALCASIVGIMFTINNQFVFWLGSGCCLILAVLLFFAKTDAPSSATVANAVGANHSAFSLKLALELFRQPKLWFLSLYVIGVSCTYDVFDQQFANFFTSFFATGEQGTRVFGYVTTMGELLNASIMFFAPLIINRIGGKNALLLAGTIMSVRIIGSSFATSALEVVILKTLHMFEVPFLLVGCFKYITSQFEVRFSATIYLVCFCFFKQLAMIFMSVLAGNMYESIGFQGAYLVLGLVALGFTLISVFTLSGPGPLSLLRRQVNEVA

SEQ ID NO: 6 - protein, 417 aa, Klebsiella pneumoniae
  REGION 1..417: note = MISC_FEATURE - Lactose permease, GenBank ID: WP_152280604.1
MYYLKNTNFWMFGLFFFFYFFIMGAYFPFFPIWLHDINHISKSDTGIIFAAISLFSLLFQPLFGLLSDKLGLRKYLLWIITGMLVMFAPFFIFIFGPLLQYNILVGSIVGGIYLGFCFNAGAPAVEAFIEKVSRRSNFEFGRARMFGCVGWALCASIVGIMFTINNQFVFWLGSGCALILAVLLFFAKTDAPSSATVANAVGANHSAFSLKLALELFRQPKLWFLSLYVIGVSCTYDVFDQQFANFFTSFFATGEQGTRVFGYVTTMGELLNASIMFFAPLIINRIGGKNALLLAGTIMSVRIIGSSFATSALEVVILKTLHMFEVPFLLVGCFKYITSQFEVRFSATIYLVCFCFFKQLAMIFMSILAGNMYESIGFQGAYLVLGLVALGFTLISVFTLSGPGPLSLLRRKVNEVA

SEQ ID NO: 7 - protein, 417 aa, Cronobacter sakazakii
  REGION 1..417: note = MISC_FEATURE - Lactose permease, GenBank ID: EGT4952364.1
MYYLKNTNFWMFGLFFFFYFFIMGAYFPFFPIWLHDINHISKSDTGIIFAAISLFSLLFQPLFGLLSDKLGLRKYLLWIITGMLVMFAPFFIFIFGPLLQYNILVGSIVGGIYLGFCFNAGAPAVEAFIEKVSRRSNFEYGRARMFGCVGWALCASIVGIMFTINNQFVFWLGSGCAFILAVLLFFAKTDAPSSATVANAVGANHSAFSLKLALELFRQPKLWFLSLYVIGVSCTYDVFDQQFANFFTSFFATGEQGTRVFGYVTTMGELLNASIMFFAPLIINRIGGKNALLLAGTIMSVRIIGSSFATSALEVVILKTLHMLEVPFLLVGCFKYITSQFEVRFSATIYLVCFCFFKQLAMIFMSILAGNMYESIGFQGAYLVLGLVALGFTLISVFTLSGPGPLSLLRRQVNEVA

SEQ ID NO: 8 - protein, 417 aa, Citrobacter freundii
  REGION 1..417: note = MISC_FEATURE - Lactose permease, GenBank ID NO: WP_134216118.1
MYYLKNTNFWMFGFFFFFYFFIMGAYFPFFPIWLHEVNHISKGDTGIIFACISLFSLLFQPIFGLLSDKLGLRKHLLWVITGMLVMFAPFFIYVFGPLLQVNILLGSIVGGIYLGFIYNAGAPAIEAYIEKASRRSNFEFGRARMFGCVGWALCASIAGIMFTINNQFVFWLGSGCAVILALLLLFSKTDAPSSAKVADAVGANNSAFSLKLALELFKQPKLWFLSLYVVGVSCTYDVFDQQFANFFTSLFATGEQGTRVFGYVTTMGELLNASIMFFAPLIVNRIGGKNALLLAGTIMSVRIIGSSFATTALEVVILKTLHMFEIPFLIVGCFKYITSQFEVRFSATIYLVCFCFFKQLAMIFMSVLAGKMYESIGFQGAYLVLGIIALSFTLISVFTLSGPGPFSLLRRRESVAL

SEQ ID NO: 9 - protein, 418 aa, Citrobacter freundii
  REGION 1..418: note = MISC_FEATURE - Lactose permease, GenBank ID: EDI1749185.1
MYYLKNTNFWMLGFFFFFYFFIMGAYFPFFPIWLHDINHISKGDTGIIFACISLFSLLFQPVFGLMSDKLGLRKNLLWIITGMLVMFAPFFIYVFGPLLHFNILLGSIVGGIYLGFIYNAGAPAIEAYIEKASRRSHFEFGRARMFGCVGWALCASIVGVMFTINNEFVFWLGSGCAVILALLLFFSRTDSGSSAVVADAVGANSSPFSLKLALELFKQPKLWFLSLYVVGVSCTYDVFDQQFANFFTSFFASGEQGTRVFGYVTTMGELLNACIMFFAPFIVNRIGGKNALLMAGIIMSVRIIGSSFATTAGEVVILKTLHMFEIPFLIVGCFKYITSQFEVRFSATIYLVCFCFFKQLAMIFMSVLAGNMYEKIGFQGAYLVLGLIALVFTLISVATLSGAGPLAVMRVSQQKSAS

SEQ ID NO: 10 - protein, 423 aa, Rouxiella badensis
  REGION 1..423: note = MISC_FEATURE - Lactose permease, GenBank ID: WP_084912833.1
MYYIKNTNFWTFGLFFFFYFFIMGAYFPFFPIWLHDINHINQSDTGIIFASISFFSLVFQPLFGLLSDKLGLKKHLLWIITGMLVMFAPFFIYVFGPLLKTNILLGSIVGGVYLGFIYNGGAPAIEAYIEKVSRRSSFEFGRARMFGCVGWAICASVVGIMFTINSQFVFWLGSGCAVILAVLLLVAKPAVGATAKVANELGANSKPFSLRLAAELLKDKKLWFLGLYVVGVSCTYEVFDQQFANFFTSFFSSADEGTRVFGYITTMGELLNALVMFFAPLIVNRIGGKNALLLAGIIMSVRIIGSSFASTPVEVVVLKTLHMFEVPFLIVGCFKYITSVFEVRFSATIYLVCFCFFKQIAMIFMSVFAGDMYGKIGFHGTYLILGLIALAFTLLSVFTLSGRGPLQAFKPVARTVEKPVSNV

SEQ ID NO: 11 - protein, 417 aa, Serratia marcescens
  REGION 1..417: note = MISC_FEATURE - Lactose permease, GenBank ID: WP_103826752.1
MYYLKNTNFWMFGLFFFFYFFFMGAYFPFFPIWLHDINHISKSDTGIIFAAISLFSLLFQPLFGLLSDKLGLRKYLLWIITGMLVMFAPFFIFIFGPLLQYNILVGSIVGGIYLGFCFNAGAPAVEAFIEKVSRRSNFEYGRARMFGCVGWALCASIVGIMFTINNQFVFWLGSGCAFILAVLLFFAKTDAPSSATVANAVGANHSAFSLKLALELFRQPKLWFLSLYVIGVSCTYDVFDQQFANFFTSFFATGEQGTRVFGYVTTMGELLNASIMFFAPLIINRIGGKNALLLAGTIMSVRIIGSSFATSALEVVILKTLHMFEVPFLLVGCFKYITSQFEVRFSATIYLVCFCFFKQLAMIFMSILAGNMYESIGFQGAYLVLGLVALGFTLISVFTLSGPGPLSLLRRQVNEVA

SEQ ID NO: 12 - protein, 420 aa, Serratia fonticola
  REGION 1..420: note = MISC_FEATURE - Lactose permease, GenBank ID: WP_021804673.1
MYYLKNTNFWMFGLFFFFYFFIMGAYFPFFPIWLHDINQVSKSDTGIIFASISFFSLLFQPIFGLLSDKLGLRKHLLWIITGMLILFAPFFIYVFGPLLRTNIVLGSIAGGIYLGFIYNGGAPAIEAYIEKVSRRSSFEFGRARLFGCVGWALCASVVGIMFTINNEFVFWLGSGCAVILAILLLIAKPEAGSTAQVADKLGANSKPFNLRLAFELLKDSKLWFLALYVVGVSCTYDVFDQQFANFFTSFFSSSEQGTRVFGYVTTMGELLNASIMFFAPLIVNRIGGKNALLLAGLIMSIRIIGSSFASTPVEVVILKTLHMFEIPFLIVGCFKYITSVFEVRFSATIYLVCFCFFKQLAIMFMSVFAGNMYEKVGFHGTYLILGLIALSFTLISVFTLSGRGPLSAFKPEPEAVRNMA

SEQ ID NO: 13 - protein, 423 aa, Rouxiella silvae
  REGION 1..423: note = MISC_FEATURE - Lactose permease, GenBank ID: WP_084984472.1
MNYMRNTNFWTFGLFFFFYFFIMGAYFPFFPIWLHDINHINQSDTGIIFACISFFSLVFQPLFGLLSDKLGLKKHLLWIITGMLVLFAPFFIYVFGPLLQTNILLGSIVGGIYLGFIYNGGAPAIEAYIEKVSRRSSFEFGRARMFGCVGWAICASVVGIMFTINNQFVFWLGSGCAVILAILLLIAKPAVGASAKVANELGANSKPFNLRLAAELLKDKKLWFLGLYVVGVSCTYEVFDQQFANFFTSFFSSADQGTRVFGYITTLGELLNAFIMFFAPAIVNRIGGKNALLLAGAIMSVRIIGSSFASTPVEVVLLKTLHMFEIPFLIVGCFKYITSVFEVRFSATIYLVCFCFFKQIAMIFMSIFAGEMYGKVGFHGTYLILGLIALAFTLVSIFTLTGRGPLQAFKPSQRVSETRPVSP

SEQ ID NO: 14 - protein, 412 aa, Aeromonas sp.
  REGION 1..412: note = MISC_FEATURE - Lactose permease, GenBank ID: WP_199428647.1
MFGAIFFLYFFIMGAYFPFFPIWLHDVNGISKAETGIVFGTISLFALLFQPVFGLVSDKLGLKKHLLWIIIGLLVFFAPFFLYVLAPLLKLNIYLGSIVGGMYIGFVFAGGAPAIEAYVEKVSRSSQFEFGRARMFGAIGWALCASIVGIMFTINNEFVFWMGSAFAVVMAVLFYFINPGRGSTSEVLDSIGANQKGFSIKLAMRLLKQAKFWFFTLYVIGVACTYDVFDQQFANFFTAFFDTKEEGTRYFGYVTTMGELLNASIMFFAPLIVNRIGGKNALLIAGTIMSIRIIGSSFAETVSHVILLKTLHMLEVPFLLVGTFKYITSEFDVRFSATIWLIGYQFFKQLATMFMSVWAGSMYEVMGFRETYLVLGIIAATFTFISIFTLSGMGPLGLLKGRTMVSKKTASY

SEQ ID NO: 15 - protein, 425 aa, Yersinia pestis
  REGION 1..425: note = MISC_FEATURE - Lactose permease, GenBank ID: WP_046596210.1
MKISKTGNYTILFTVSFFIMASYYPFFPIWLHDINNLSKTDTGIVFGSISLFALAFQPIMGPLSDKLGLRKTLMWIIVGLLVLFAPFFIYVFSPLLKYNIFIGAIVGGCYLGFVFTGGSHAIEAYIEKVSRHSNFEYGRVRMFGCIGWALCATVVGILYTVNNQLIFWMASGCALILAVLLFFARPDRQSTAFVVDTLGANKAVFNLKNALALLRKRELWFFVMYIVGVACIYDVFDQQFANFFTSFFATKQQGTEIFGFVTTGGEILNATVMFFAPVIIARIGSKNALLLAGTIMSVRILGSAFATTATQVVFLKMLHMFEVPFLLVGSFKYITQVFEVRFSATVYLIGFCFSKQLSMMFMSVFAGRMYGSMGYQDTYMVLGVIVLSFTLISAFTLSGRSAVANLASRLKEDPSVTPPVSQPQS

SEQ ID NO: 16 - protein, 587 aa, Kluyveromyces lactis
  REGION 1..587: note = MISC_FEATURE - Lactose permease, GenBank ID: XP_452193.1
MADHSSSSSSLQKKPINTIEHKDTLGNDRDHKEALNSDNDNTSGLKINGVPIEDAREEVLLPGYLSKQYYKLYGLCFITYLCATMQGYDGALMGSIYTEDAYLKYYHLDINSSSGTGLVFSIFNVGQICGAFFVPLMDWKGRKPAILIGCLGVVIGAIISSLTTTKSALIGGRWFVAFFATIANAAAPTYCAEVAPAHLRGKVAGLYNTLWSVGSIVAAFSTYGTNKNFPNSSKAFKIPLYLQMMFPGLVCIFGWLIPESPRWLVGVGREEEAREFIIKYHLNGDRTHPLLDMEMAEIIESFHGTDLSNPLEMLDVRSLFRTRSDRYRAMLVILMAWFGQFSGNNVCSYYLPTMLRNVGMKSVSLNVLMNGVYSIVTWISSICGAFFIDKIGRREGFLGSISGAALALTGLSICTARYEKTKKKSASNGALVFIYLFGGIFSFAFTPMQSMYSTEVSTNLTRSKAQLLNFVVSGVAQFVNQFATPKAMKNIKYWFYVFYVFFDIFEFIVIYFFFVETKGRSLEELEVVFEAPNPRKASVDQAFLAQVRATLVQRNDVRVANAQNLKEQEPLKSDADHVEKLSEAESV

SEQ ID NO: 17 - protein, 262 aa, Chromobacterium violaceum
  REGION 1..262: note = MISC_FEATURE - cvb3galT, -1,3-galactosyltransferase, GeneBank ID:: WP_080969100.1
MDTIMIKRPLVSVILPVNKNNPHLEEAIQSIKNQTYKELELIIIANNCEDNFYSLLLKYQDQKTKIIRTSIKYLPFSLNLGVHLSQGEYIARMDSDDISVLDRIEKQVKRFLNTPELSILGSNVEYINEASESIGYSNYPLDHSSIVNSFPFRCNLAHPTIMVKKEVITTLGGYMYGSLSEDYDLWIRASRHGNFKFSNIDEPLLKYRIHKGQATNKSNAYNIFAFDSSLKIREFLLNGNVQYLLGAARGFFAFLYVRFIKK

SEQ ID NO: 18 - protein, 439 aa, synthetic construct
  REGION 1..439: note = galTK, -1,3-galactosyltransferase, homologous to GeneBank ID: BD182026.1
MISVYIISLKESQRRLDTEKLVLESNEKFKGRCVFQIFDAISPKHEDFEKFVQELYDSSSLLKSDWFHSDYCYQELLPQEFGCYLSHYLLWKECVKLNQPVVILEDDVALESNFMQALEDCLKSPFDFVRLYGHYWGGHKTNLCALPVYTETEEAEASIEKTPIENYEVTSPPPPNPTRDTQQDFITETQQDPKELSEPCKIAPQKISFNQVVFKKIKRKLNRFIGSILARTEVYKNIVAKYDDLTTKYDDLTTKYDDLTTKYDDLTTKYDDLNKNIAEKYDELMGKYESLLAKEVNIKETFWESRADSEKEALFLDHFYLTSVYVATTAGYYLTPKGAKTFIEATERFKIIEPVDMFINNPTYHDIANFTYVPCPVSLNKHAFNSTIQNAKKPDISLKPPKKSYFDNLFYHKFNARKCLKAFNKYSKQYAPLKTPKEV

SEQ ID NO: 19 - protein, 273 aa, Helicobacter pylori
  REGION 1..273: note = MISC_FEATURE - -1,4-gal-transferase, GalT, GenBank ID: WP_001262061.1
MRVFAISLNQKVCDTFGLVFRDTTTLLNSINATHHQAQIFDAIYSKTFEGGLHPLVKKHLHPYFITQNIKDMGITTNLISEVSKFYYALKYHAKFMSLGELGCYASHYSLWEKCIELNEAICILEDDITLKEDFKEGLDFLEKHIQELGYIRLMHLLYDASVKSEPLSHKNHEIQERVGIIKAYSEGVGTQGYVITPKIAKVFLKCSRKWVVPVDTIMDATFIHGVKNLVLQPFVIADDEQISTIARKEEPYSPKIALMRELHFKYLKYWQFV

SEQ ID NO: 20 - protein, 332 aa, Neisseria meningitidis
  REGION 1..332: note = MISC_FEATURE - lgtA, -1,3-N-acetyl-glucosaminyltransferase, GeneBank ID: WP_033911473.1
MQPLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRPEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGAWLDFAADGRMRRLFTLRQYFGILHRLLKNR

SEQ ID NO: 21 - protein, 334 aa, Pasteurella multocida
  REGION 1..334: note = MISC_FEATURE - pmnagT, -1,3-N-acetylglucosaminyl-transferase, GeneBankID: WP_014390683.1
MENKPLVSVLICAYNVEKYIEECINAVINQTYKNLEIIIVNDGSSDNTYFLLKKLAEKDNRIKILNFNNHIGIISALNEGLKEIAGEYIARTDSDDITKPDWIEKILTCMQNDPKIIAMGSYLTVLSEENNGSVLANHHKNKVEWKNPLEHKDIVEKMLFGNPIHNNSMVMRSEIYTKYHLIYDPDYHYAEDYKFWLEVSRIGKLANYPESLVYYRLHRNQTSSIHNSQQEINGKKLRLQALNYYLKDLGIDYQLPEKFLFKDIALLQEIFYERGMFRENIIRRIIYECYLSLGEYNYKDIYYFLINKNNFLSIKDKFKIIKKYLRPDKYSSTY

SEQ ID NO: 22 - protein, 330 aa, Haemophilus ducreyi
  REGION 1..330: note = MISC_FEATURE - HD0466, glycosyltransferase family 2 protein, GeneBank ID: WP_010944479.1
MTTLVSVLICAYNVEKYIDECLNAVIAQTYKNLEIIVVNDGSTDGTLAKLRQFEAKDPRVKIIDNIVNQGTSKSLNIGIQYCQGEIIARTDSDDIVDIHWIETLMRELDNSPETIAISAYLEFLAEKGNGSKLSRSRKHGKNAENPISSEAISQRMLFGNPVHNNVALVRRKVFSEYGLRFDPDYIHAEDYKFWFEVSKLGKMRTYPKALVKYRLHATQVSSAYNQKQRSIAKKIKREAISHYLQQYGIQLPEKLTIHDLFSIFSPQIELSLTVANKQELFWSLATSLSEYHFRDLLKIYSLDIFHQLSFKYKKRIFRKFLLPNRYPSVI

SEQ ID NO: 23 - protein, 302 aa, synthetic construct
  REGION 1..302: note = FutC (-1,2-fucosyl-transferase)
MAFKVVQICGGLGNQMFQYAFAKSLQKHSNTPVLLDITSFDWSDRKMQLELFPIDLPYASAKEIAIAKMQHLPKLVRDALKCMGFDRVSQEIVFEYEPKLLKPSRLTYFFGYFQDPRYFDAISPLIKQTFTLPPPPENNKNNNKKEEEYQCKLSLILAAKNSVFVHIRRGDYVGIGCQLGIDYQKKALEYMAKRVPNMELFVFCEDLEFTQNLDLGYPFMDMTTRDKEEEAYWDMLLMQSCQHGIIANSTYSWWAAYLIENPEKIIIGPKHWLFGHENILCKEWVKIESHFEVKSQKYNALG

SEQ ID NO: 24 - protein, 292 aa, Sulfuriflexus mobilis
  REGION 1..292: note = MISC_FEATURE - Smob, -1,2-fucosyltransferase, GeneBank ID: WP_126455392.1
MIISQIIGGLGNQMFQYAAGRALSLVRGQPLLLDVTGFAGYGLHQGFELQRVFDCPIGIATEEDVRGILGWQFSAGIRRIVARPGMAAFRRKGFIVEPHFHYWPEIKNVPRDCYLLGYWQSERYFRAATADIRADFSFKSPLVNRNAETAAQIDQVNAISLHMRRGDYVNNPKTSATHGLCSLDYYQAAIKFVSERVEEPFFFIFSDDIAWVKANLKLDFPCQYVDHNHGAESFNDMHLMSLCQHHIIANSSFSWWGAWLNSDPKKIVLAPKKWFANKNNIKDLFPPGWVSL

SEQ ID NO: 25 - protein, 292 aa, Methylobacter tundripaludum
  REGION 1..292: note = MISC_FEATURE - Mtun, -1,2-fucosyltransferase, GeneBank ID: WP_031437198.1
MVITHLIGGLGNQMFQYAAGRAVSLERGVSLSLDISGFANYGLHQGFELQRIFNCTAEIANEADVRGILGWQSSPRIRQLLSRQNMAIFRREGFVVEPHFHYWQGIKSVPRDCYLTGYWQSEQYFLEAAAQIRADFTFKLPLDNQNIELAKQINAVNAVSLHVRRGDYANTPETTATHGLCSLDYYRVAIRHIAEQVQQPHFFVFSDDIAWVKNNLSIDFPCQYVDHNQGAESYNDMRLMSMCRHHIIANSSFSWWGAWLNPNVNKIVVAPSRWFAKQTDVRDLLPQGWIKQ

SEQ ID NO: 26 - protein, 425 aa, Helicobacter pylori
  REGION 1..425: note = MISC_FEATURE - FutA, -1,3-fucosyl-transferase, GenBank ID: NP_207177.1
MFQPLLDAFIESASIEKMASKSPPPPLKIAVANWWGDEEIKEFKKSVLYFILSQRYAITLHQNPNEFSDLVFSNPLGAARKILSYQNTKRVFYTGENESPNFNLFDYAIGFDELDFNDRYLRMPLYYAHLHYKAELVNDTTAPYKLKDNSLYALKKPSHHFKENHPNLCAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHKFSTSFMWEYDLHKPLVSIDDLRVNYDDLRVNYDRLLQNASPLLELSQNTTFKIYRKAYQKSLPLLRAVRKLVKKLGL

SEQ ID NO: 27 - protein, 478 aa, Helicobacter pylori
  REGION 1..478: note = MISC_FEATURE - FucT, alpha1,3-fucosyltransferase, GenBank ID NO: AAB81031.1
MFQPLLDAYVESASIEKMASKSPPPLKIAVANWWGDEEIKEFKNSVLYFILSQRYTITLHQNPNEFSDLVFGNPLGSARKILSYQNAKRVFYTGENESPNFNLFDYAIGFDELDFNDRYLRMPLYYDRLHHKAESVNDTTAPYKLKDNSLYALKKPSHCFKEKHPNLCAVVNDESDPLKRGFASFVASNPNAPIRNAFYDALNSIEPVTGGGSVRNTLGYNVKNKNEFLSQYKFNLCFENTQGYGYVTEKIIDAYFSHTIPIYWGSPSVAKDFNPKSFVNVHDFKNFDEAIDYIKYLHTHKNAYLDMLYENPLNTLDGKAYFYQNLSFKKILAFFKTILENDTIYHDNPFIFCRDLNEPLVTIDDLRVNYDDLRVNYDDLRINYDDLRVNYDDLRINYDDLRVNYDDLRVNYDDLRINYDDLRVNYDDLRVNYERLLSKATPLLELSQNTTSKIYRKAYQKSLPLLRAIRRWVKKLGL

SEQ ID NO: 28 - protein, 432 aa, Helicobacter pylori
  REGION 1..432: note = MISC_FEATURE - FucTIII, alpha-1,4 fucosyltransferase, GenBank ID: AAR88243.1
MFQPLLDAYIDSTRLDETDYKPPLKIAVANWWGGVEEFKKSTLYFILSQRYTITLHRNPDKPADIVFGNPLGSARKILSYQNAKRVFYTGENEVPNFNLFDYAIGFDELDFNDRYLRMPLYYAHLHYEAELVNDTTSPYKIKDNSLYALKKPSHHFKENHPNLCAVVNNESDPLKRGFASFVASNPNAPKRNAFYDALNSIEPVTGGGSVKNTLGYNVKNKNEFLSQYKFNLCFENSQGYGYVTEKILDAYFSHTIPIYWGSPSVAKDFNPKSFVNVHDFNNFDEAIDHVRYLHTHPNAYLDMLYENPLNTLDGKAYFYQNLSFKKILDFFKTILENDTIYHCDAHNYSALHRDLNEPLVSIDDLRINYDDLRINYDDLRINYDDLRINYERLLQNASPLLELSQNTSFKIYRKAYQKSLPLLRAIRRWVKK

SEQ ID NO: 29 - protein, 483 aa, synthetic construct
  REGION 1..483: note = Pd2 (-1,3-sialyl-transferase)
MCNSDNTSLKETVSSNSADVVETETYQLTPIDAPSSFLSHSWEQTCGTPILNESDKQAISFDFVAPELKQDEKYCFTFKGITGDHRYITNTTLTVVAPTLEVYIDHASLPSLQQLIHIIQAKDEYPSNQRFVSWKRVTVDADNANKLNIHTYPLKGNNTSPEMVAAIDEYAQSKNRLNIEFYTNTAHVFNNLPPIIQPLYNNEKVKISHISLYDDGSSEYVSLYQWKDTPNKIETLEGEVSLLANYLAGTSPDAPKGMGNRYNWHKLYDTDYYFLREDYLDVEANLHDLRDYLGSSAKQMPWDEFAKLSDSQQTLFLDIVGFDKEQLQQQYSQSPLPNFIFTGTTTWAGGETKEYYAQQQVNVINNAINETSPYYLGKDYDLFFKGHPAGGVINDIILGSFPDMINIPAKISFEVLMMTDMLPDTVAGIASSLYFTIPADKVNFIVFTSSDTITDREEALKSPLVQVMLTLGIVKEKDVLFWA

SEQ ID NO: 30 - protein, 342 aa, synthetic construct
  REGION 1..342: note = Nst (-1,3-sialyl-transferase)
MERNAVSLLKEKLFNEEGEPVNLIFCYTILQMKVAERIMAQHPGERFYVVLMSENRNEKYDYYFNQIKDKAERAYFFHLPYGLNKSFNFIPTMAELKVKSMLLPKVKRIYLASLEKVSIAAFLSTYPDAEIKTFDDGTGNLIQSSSYLGDEFSVNGTIKRNFARMMIGDWSIAKTRNASDEHYTIFKGLKNIMDDGRRKMTYLPLFDASELKTGDETGGTVRILLGSPDKEMKEISEKAAKNFKIQYVAPHPRQTYGLSGVTTLNSPYVIEDYILREIKKNPHTRYEIYTFFSGAALTMKDFPNVHVYALKPASLPEDYWLKPVYALFTQSGIPILTFDDKN

SEQ ID NO: 31 - protein, 373 aa, Escherichia coli [sequence omitted]

SEQ ID NO: 32 - protein, 373 aa, Escherichia sp. [sequence omitted]

SEQ ID NO: 33 - protein, 321 aa, Escherichia coli
  REGION 1..321: note = MISC_FEATURE - WcaG, GDP-L-fucose synthase
MSKQRVFIAGHRGMVGSAIRRQLEQRGDVELVLRTRDELNLLDSRAVHDFFASERIDQVYLAAAKVGGIVANNTYPADFIYQNMMIESNIIHAAHQNDVNKLLFLGSSCIYPKLAKQPMAESELLQGTLEPTNEPYAIAKIAGIKLCESYNRQYGRDYRSVMPTNLYGPHDNFHPSNSHVIPALLRRFHEATAQNAPDVVVWGSGTPMREFLHVDDMAAASIHVMELAHEVWLENTQPMLSHINVGTGVDCTIRELAQTIAKVVGYKGRVVFDASKPDGTPRKLLDVTRLHQLGWYHEISLEAGLASTYQWFLENQDRFRG

SEQ ID NO: 34 - protein, 321 aa, Escherichia sp.; identical to SEQ ID NO: 33
  REGION 1..321: note = MISC_FEATURE - WcaG, GDP-L-fucose synthase, GenBank ID: WP_000043654.1

SEQ ID NO: 35 - protein, 159 aa, Escherichia coli
  REGION 1..159: note = MISC_FEATURE - WcaH, GDP-mannose mannosyl hydrolase, GenBank ID: NP_416555.2
MFLRQEDFATVVRSTPLVSLDFIVENSRGEFLLGKRTNRPAQGYWFVPGGRVQKDETLEAAFERLTMAELGLRLPITAGQFYGVWQHFYDDNFSGTDFTTHYVVLGFRFRVSEEELLLPDEQHDDYRWLTSDALLASDNVHANSRAYFLAEKRTGVPGL

SEQ ID NO: 36 - protein, 159 aa, Escherichia sp.; identical to SEQ ID NO: 35
  REGION 1..159: note = MISC_FEATURE - WcaH, GDP-mannose mannosyl hydrolase, GenBank ID: WP_001393539.1

SEQ ID NO: 37 - protein, 407 aa, Escherichia coli
  REGION 1..407: note = MISC_FEATURE - WcaI, colanic acid biosynthesis fucosyltransferase, GenBank ID: NP_416554.1
MKILVYGINYSPELTGIGKYTGEMVEWLAAQGHEVRVITAPPYYPQWQVGENYSAWRYKREEGAATVWRCPLYVPKQPSTLKRLLHLGSFAVSSFFPLMAQRRWKPDRIIGVVPTLFCAPGMRLLAKLSGARTVLHIQDYEVDAMLGLGLAGKGKGGKVAQLATAFERSGLHNVDNVSTISRSMMNKAIEKGVAAENVIFFPNWSEIARFQHVADADVDALRNQLDLPDNKKIILYSGNIGEKQGLENVIEAADRLRDEPLIFAIVGQGGGKARLEKMAQQRGLRNMQFFPLQSYDALPALLKMGDCHLVVQKRGAADAVLPSKLTNILAVGGNAVITAEAYTELGQLCETFPGIAVCVEPESVEALVAGIRQALLLPKHNTVAREYAERTLDKENVLRQFINDIRG

SEQ ID NO: 38 - protein, 407 aa, Escherichia sp.; identical to SEQ ID NO: 37
  REGION 1..407: note = MISC_FEATURE - WcaI, colanic acid biosynthesis fucosyltransferase , GenBank ID: WP_000699693.1

SEQ ID NO: 39 - protein, 478 aa, Escherichia coli
  REGION 1..478: note = MISC_FEATURE - ManC, mannose-1-phosphate guanylyltransferase, GenBank ID: NP_416553.1
MAQSKLYPVVMAGGSGSRLWPLSRVLYPKQFLCLKGDLTMLQTTICRLNGVECESPVVICNEQHRFIVAEQLRQLNKLTENIILEPAGRNTAPAIALAALAAKRHSPESDPLMLVLAADHVIADEDAFRAAVRNAMPYAEAGKLVTFGIVPDLPETGYGYIRRGEVSAGEQDMVAFEVAQFVEKPNLETAQAYVASGEYYWNSGMFLFRAGRYLEELKKYRPDILDACEKAMSAVDPDLNFIRVDEEAFLACPEESVDYAVMERTADAVVVPMDAGWSDVGSWSSLWEISAHTAEGNVCHGDVINHKTENSYVYAESGLVTTVGVKDLVVVQTKDAVLIADRNAVQDVKKVVEQIKADGRHEHRVHREVYRPWGKYDSIDAGDRYQVKRITVKPGEGLSVQMHHHRAEHWVVVAGTAKVTIDGDIKLLGENESIYIPLGATHCLENPGKIPLDLIEVRSGSYLEEDDVVRFADRYGRV

SEQ ID NO: 40 - protein, 478 aa, Escherichia sp.; identical to SEQ ID NO: 39
  REGION 1..478: note = MISC_FEATURE - ManC, mannose-1-phosphate guanyltransferase, GenBank ID: WP_000079274.1

SEQ ID NO: 41 - protein, 456 aa, Escherichia coli
  REGION 1..456: note = MISC_FEATURE - ManB, phosphomannomutase, GenBank ID: NP_416552.1
MKKLTCFKAYDIRGKLGEELNEDIAWRIGRAYGEFLKPKTIVLGGDVRLTSETLKLALAKGLQDAGVDVLDIGMSGTEEIYFATFHLGVDGGIEVTASHNPMDYNGMKLVREGARPISGDTGLRDVQRLAEANDFPPVDETKRGRYQQINLRDAYVDHLFGYINVKNLTPLKLVINSGNGAAGPVVDAIEARFKALGAPVELIKVHNTPDGNFPNGIPNPLLPECRDDTRNAVIKHGADMGIAFDGDFDRCFLFDEKGQFIEGYYIVGLLAEAFLEKNPGAKIIHDPRLSWNTVDVVTAAGGTPVMSKTGHAFIKERMRKEDAIYGGEMSAHHYFRDFAYCDSGMIPWLLVAELVCLKDKTLGELVRDRMAAFPASGEINSKLAQPVEAINRVEQHFSREALAVDRTDGISMTFADWRFNLRTSNTEPVVRLNVESRGDVPLMEARTRTLLTLLNE

SEQ ID NO: 42 - protein, 456 aa, Escherichia sp.; identical to SEQ ID NO: 41
  REGION 1..456: note = MISC_FEATURE - ManB, phosphomannomutase, GenBank ID: WP_001350528.1

SEQ ID NO: 43 - protein, 546 aa, Escherichia coli
  REGION 1..546: note = MISC_FEATURE - pgm, Phosphoglucomutase, GenBank ID: NP_415214.1
MAIHNRAGQPAQQSDLINVAQLTAQYYVLKPEAGNAEHAVKFGTSGHRGSAARHSFNEPHILAIAQAIAEERAKNGITGPCYVGKDTHALSEPAFISVLEVLAANGVDVIVQENNGFTPTPAVSNAILVHNKKGGPLADGIVITPSHNPPEDGGIKYNPPNGGPADTNVTKVVEDRANALLADGLKGVKRISLDEAMASGHVKEQDLVQPFVEGLADIVDMAAIQKAGLTLGVDPLGGSGIEYWKRIGEYYNLNLTIVNDQVDQTFRFMHLDKDGAIRMDCSSECAMAGLLALRDKFDLAFANDPDYDRHGIVTPAGLMNPNHYLAVAINYLFQHRPQWGKDVAVGKTLVSSAMIDRVVNDLGRKLVEVPVGFKWFVDGLFDGSFGFGGEESAGASFLRFDGTPWSTDKDGIIMCLLAAEITAVTGKNPQEHYNELAKRFGAPSYNRLQAAATSAQKAALSKLSPEMVSASTLAGDPITARLTAAPGNGASIGGLKVMTDNGWFAARPSGTEDAYKIYCESFLGEEHRKQIEKEAVEIVSEVLKNA

SEQ ID NO: 44 - protein, 553 aa, Escherichia coli
  REGION 1..553: note = MISC_FEATURE - pgm, phosphoglucomutase, GenBank ID: WP_001396326.1
MQTKDKAMAIHNRAGQPAQQSDLINVAQLTAQYYVLKPEAGNAEHAVKFGTSGHRGSAARHSFNEPHILAIAQAIAEERAKNGITGPCYVGKDTHALSEPAFISVLEVLAANGVDVIVQENNGFTPTPAVSNAILVHNKKGGPLADGIVITPSHNPPEDGGIKYNPPNGGPADTNVTKVVEDRANALLADGLKGVKRISLDEAMASGHVKEQDLVQPFVEGLADIVDMAAIQKAGLTLGVDPLGGSGIEYWKRIGEYYNLNLTIVNDQVDQTFRFMHLDKDGAIRMDCSSECAMAGLLALRDKFDLAFANDPDYDRHGIVTPAGLMNPNHYLAVAINYLFQHRPQWGKDVAVGKTLVSSAMIDRVVNDLGRKLVEVPVGFKWFVDGLFDGSFGFGGEESAGASFLRFDGTPWSTDKDGIIMCLLAAEITAVTGKNPQEHYNELAKRFGAPSYNRLQAAATSAQKAALSKLSPEMVSASTLAGDPITARLTAAPGNGASIGGLKVMTDNGWFAARPSGTEDAYKIYCESFLGEEHRKQIEKEAVEIVSEVLKNA

SEQ ID NO: 45 - protein, 302 aa, Escherichia coli
  REGION 1..302: note = MISC_FEATURE - GalU, UTP-glucose-1-phosphate uridylyltransferase, GenBank: NP_415752.1
MAAINTKVKKAVIPVAGLGTRMLPATKAIPKEMLPLVDKPLIQYVVNECIAAGITEIVLVTHSSKNSIENHFDTSFELEAMLEKRVKRQLLDEVQSICPPHVTIMQVRQGLAKGLGHAVLCAHPVVGDEPVAVILPDVILDEYESDLSQDNLAEMIRRFDETGHSQIMVEPVADVTAYGVVDCKGVELAPGESVPMVGVVEKPKADVAPSNLAIVGRYVLSADIWPLLAKTPPGAGDEIQLTDAIDMLIEKETVEAYHMKGKSHDCGNKLGYMQAFVEYGIRHNTLGTEFKAWLEEEMGIKK

SEQ ID NO: 46 - protein, 302 aa, Escherichia sp.; identical to SEQ ID NO: 45
  REGION 1..302: note = MISC_FEATURE - GalU, UTP-glucose-1-phosphate uridylyltransferase, WP_000718995.1

SEQ ID NO: 47 - protein, 338 aa, Escherichia coli K-12
  REGION 1..338: note = MISC_FEATURE - GalE, UDP-glucose 4-epimerase, GenBank ID: NP_415280.3
MRVLVTGGSGYIGSHTCVQLLQNGHDVIILDNLCNSKRSVLPVIERLGGKHPTFVEGDIRNEALMTEILHDHAIDTVIHFAGLKAVGESVQKPLEYYDNNVNGTLRLISAMRAANVKNFIFSSSATVYGDQPKIPYVESFPTGTPQSPYGKSKLMVEQILTDLQKAQPDWSIALLRYFNPVGAHPSGDMGEDPQGIPNNLMPYIAQVAVGRRDSLAIFGNDYPTEDGTGVRDYIHVMDLADGHVVAMEKLANKPGVHIYNLGAGVGNSVLDVVNAFSKACGKPVNYHFAPRREGDLPAYWADASKADRELNWRVTRTLDEMAQDTWHWQSRHPQGYPD

SEQ ID NO: 48 - protein, 338 aa, Escherichia sp.; identical to SEQ ID NO: 47
  REGION 1..338: note = MISC_FEATURE - GalE, UDP-glucose 4-epimerase, GenBank ID: WP_001265438.1

SEQ ID NO: 49 - protein, 445 aa, Escherichia coli K-12
  REGION 1..445: note = MISC_FEATURE - GlmM, Phosphoglucosamine mutase, GenBank ID: NP_417643.1
MSNRKYFGTDGIRGRVGDAPITPDFVLKLGWAAGKVLARHGSRKIIIGKDTRISGYMLESALEAGLAAAGLSALFTGPMPTPAVAYLTRTFRAEAGIVISASHNPFYDNGIKFFSIDGTKLPDAVEEAIEAEMEKEISCVDSAELGKASRIVDAAGRYIEFCKATFPNELSLSELKIVVDCANGATYHIAPNVLRELGANVIAIGCEPNGVNINAEVGATDVRALQARVLAEKADLGIAFDGDGDRVIMVDHEGNKVDGDQIMYIIAREGLRQGQLRGGAVGTLMSNMGLELALKQLGIPFARAKVGDRYVLEKMQEKGWRIGAENSGHVILLDKTTTGDGIVAGLQVLAAMARNHMSLHDLCSGMKMFPQILVNVRYTAGSGDPLEHESVKAVTAEVEAALGNRGRVLLRKSGTEPLIRVMVEGEDEAQVTEFAHRIADAVKAV

SEQ ID NO: 50 - protein, 445 aa, Escherichia sp.; identical to SEQ ID NO: 49
  REGION 1..445: note = MISC_FEATURE - GlmM, Phosphoglucosamine mutase, GenBank ID: WP_000071134.1

SEQ ID NO: 51 - protein, 456 aa, Escherichia coli K-12
  REGION 1..456: note = MISC_FEATURE - GlmU, N-acetylglucosamine-1-phosphate uridyltransferase and glucosamine-1-phosphate acetyltransferase, GenBank ID: NP_418186.1
MLNNAMSVVILAAGKGTRMYSDLPKVLHTLAGKAMVQHVIDAANELGAAHVHLVYGHGGDLLKQALKDDNLNWVLQAEQLGTGHAMQQAAPFFADDEDILMLYGDVPLISVETLQRLRDAKPQGGIGLLTVKLDDPTGYGRITRENGKVTGIVEHKDATDEQRQIQEINTGILIANGADMKRWLAKLTNNNAQGEYYITDIIALAYQEGREIVAVHPQRLSEVEGVNNRLQLSRLERVYQSEQAEKLLLAGVMLRDPARFDLRGTLTHGRDVEIDTNVIIEGNVTLGHRVKIGTGCVIKNSVIGDDCEISPYTVVEDANLAAACTIGPFARLRPGAELLEGAHVGNFVEMKKARLGKGSKAGHLTYLGDAEIGDNVNIGAGTITCNYDGANKFKTIIGDDVFVGSDTQLVAPVTVGKGATIAAGTTVTRNVGENALAISRVPQTQKEGWRRPVKKK

SEQ ID NO: 52 - protein, 456 aa, Escherichia sp.; identical to SEQ ID NO: 51
  REGION 1..456: note = MISC_FEATURE - GlmU, N-acetylglucosamine-1-phosphate uridyltransferase and glucosamine-1-phosphate acetyltransferase, GenBank ID: WP_000933736.1

SEQ ID NO: 53 - protein, 609 aa, Escherichia coli K-12
  REGION 1..609: note = MISC_FEATURE - GlmS, L-glutamine--D-fructose-6-phosphate aminotransferase, GenBank ID: NP_418185.1
MCGIVGAIAQRDVAEILLEGLRRLEYRGYDSAGLAVVDAEGHMTRLRRLGKVQMLAQAAEEHPLHGGTGIAHTRWATHGEPSEVNAHPHVSEHIVVVHNGIIENHEPLREELKARGYTFVSETDTEVIAHLVNWELKQGGTLREAVLRAIPQLRGAYGTVIMDSRHPDTLLAARSGSPLVIGLGMGENFIASDQLALLPVTRRFIFLEEGDIAEITRRSVNIFDKTGAEVKRQDIESNLQYDAGDKGIYRHYMQKEIYEQPNAIKNTLTGRISHGQVDLSELGPNADELLSKVEHIQILACGTSYNSGMVSRYWFESLAGIPCDVEIASEFRYRKSAVRRNSLMITLSQSGETADTLAGLRLSKELGYLGSLAICNVPGSSLVRESDLALMTNAGTEIGVASTKAFTTQLTVLLMLVAKLSRLKGLDASIEHDIVHGLQALPSRIEQMLSQDKRIEALAEDFSDKHHALFLGRGDQYPIALEGALKLKEISYIHAEAYAAGELKHGPLALIDADMPVIVVAPNNELLEKLKSNIEEVRARGGQLYVFADQDAGFVSSDNMHIIEMPHVEEVIAPIFYTVPLQLLAYHVALIKGTDVDQPRNLAKSVTVE

SEQ ID NO: 54 - protein, 609 aa, Escherichia sp.; identical to SEQ ID NO: 53
  REGION 1..609: note = MISC_FEATURE - GlmS, L-glutamine--D-fructose-6-phosphate aminotransferase, GenBank ID: WP_000334099.1

SEQ ID NO: 55 - protein, 221 aa, Campylobacter jejuni
  REGION 1..221: note = MISC_FEATURE - NeuA, CMP-Neu5Ac synthetase, GenBank ID: AAK91728.1
MSLAIIPARGGSKGIKNKNLVLLNNKPLIYYTIKAALNAKSISKVVVSSDSDEILNYAKSQNVDILKRPISLAQDDTTSDKVLLHALKFYKDYEDVVFLQPTSPLRTNIHINEAFNLYKNSNANALISVSECDNKILKAFVCNDCGDLAGICNDEYPFMPRQKLPKTYMSNGAIYILKIKEFLNNPSFLQSKTKHFLMDESSSLDIDCLEDLKKVEQIWKK

SEQ ID NO: 56 - protein, 231 aa, Vibrio brasiliensis
  REGION 1..231: note = MISC_FEATURE - NeuA, CMP-Neu5Ac synthetase, GenBank ID: WP_006881452.1
MRVALITARGGSKGLPRKNVLNVNGLPLIGWTINAARECSYIDRVFVSTEDEEIAKISLGMGAELIERPCELAQDNSTSDEVIRHAIDWLDKHSILSDLVVLLQPTSPLRTSAHISEAIDLYDSLNNPNATIISVYEPEHSPLKAFVQLENGTLKGSYNEDAPYTRRQELPKSYLANGAIYIFSPSAFMSESQIPRRDIYPYVMSTNESEDIDSKIDLIRVEKLLRGSFYE

SEQ ID NO: 57 - protein, 346 aa, Campylobacter jejuni
  REGION 1..346: note = MISC_FEATURE - NeuB, Neu5 Ac synthase, GenBank ID: AAK91726.1
MKEIKIQNIIISEEKAPLVVPEIGINHNGSLELAKIMVDAAFSAGAKIIKHQTHIVEDEMSKAAKKVIPGNAKISIYEIMQKCALDYKDELALKEYTEKLGLVYLSTPFSRAGANRLEDMGVSAFKIGSGECNNYPLIKHIAAFKKPMIVSTGMNSIESIKPTVKILLDNEIPFVLMHTTNLYPTPHNLVRLNAMLELKKEFSCMVGLSDHTTDNLACLGAVVLGACVLERHFTDSMHRSGPDIVCSMDTKALKELIIQSEQMAIIRGNNESKKAAKQEQVTIDFAFASVVSIKDIKKGEVLSMDNIWVKRPGLGGISAAEFENILGKKALRDIENDAQLSYEDFA

SEQ ID NO: 58 - protein, 346 aa, Flavobacterium limnosediminis
  REGION 1..346: note = MISC_FEATURE - NeuB, Neu5 Ac synthase, GenBank ID: WP_023580510.1
MNPYIEIAGRKIGLDYPPLVIAEIGINHEGSLQVAKEIVDAAFRAGVEVVKHQTHIVEDEMSGAAKKVIPGNADVSIYEIMERCALDESEELELKNYVESKGMIFISTPFSRAAAERLKKFDIPAYKIGSGECNNYPLLEHIASFGKPVILSTGMNTVDSIRKAVAIFDKHNVPVALLHTTNLYPTPIHLVRFGAMMEMHNAFPDKVFGLSDHTLNNNACLGAVALGASILERHFTDHMSRTGPDIVCSMDEQATRELIVNSAEIALMRGGTKKPAAEEQVTIDFAFATVCSIAPIKKGEVFTKENIWVKRPGTGKILAEHFERLLGKTATRDIENDEQLDFSDVE

SEQ ID NO: 59 - protein, 372 aa, Campylobacter jejuni
  REGION 1..372: note = MISC_FEATURE - NeuC, UDP-GlcNAc 2-epimerase, GenBank AAK91727.1
MKKILFITGSRADYSKIKSLMYRVQNSSEFELYIFATGMHLSKNFGYTVKELYKNGFKNIYEFINYDKYYQTDKALATTIDGFSRYANELKPDLIVVHGDRIEPLAAAIVGALNNILVAHIEGGEISGTIDDSLRHAISKLAHIHLVNDEFAKRRLMQLGEDEKSIFIIGSPDLELLNDNKISLSEAKKYYDINYENYALLMFHPVTTEITSIKNQADNLVKALIQSNKNYIVIYPNNDLGFELILQSYEEFKNNPRFKLFPSLRFEYFITLLKNADFIIGNSSCILKEALYLKTAGILVGSRQNGRLGNENTLKVNANSDEILKAINTIHKKQDLFSAKLEILDSSKLFFEYLQSGDFFKLSTQKVFKDIK

SEQ ID NO: 60 - protein, 391 aa, Escherichia sp.
  REGION 1..391: note = MISC_FEATURE - NeuC, UDP-GlcNAc 2-epimerase, GenBank ID: WP_000723250.1
MKKILYVTGSRAEYGIVRRLLTMLRETPEIQLDLAVTGMHCDNAYGNTIHIIEQDNFNIIKVVDININTTSHTHILHSMSVCLNSFGDFFSNNTYDAVMVLGDRYEIFSVAIAASMHNIPLIHIHGGEKTLANYDEFIRHSITKMSKLHLTSTEEYKKRVIQLGEKPGSVFNIGSLGAENALSLHLPNKQELELKYGSLLKRYFVVVFHPETLSTQSVNDQIDELLSAISFFKNTHDFIFIGSNADTGSDIIQRKVKYFCKEYKFRYLISIRSEDYLAMIKYSCGLIGNSSSGLIEVPSLKVATINIGDRQKGRVRGASVIDVPVEKNAIVRGINISQDEKFISVVQSSSNPYFKENALINAVRIIKDFIKSKNKDYKDFYDIPECTTSYD

SEQ ID NO: 61 - protein, 391 aa, Escherichia coli S88; identical to SEQ ID NO: 60
  REGION 1..391: note = MISC_FEATURE - NeuC, UDP-GlcNAc 2-epimerase, GenBank ID: CAR04561.1

SEQ ID NO: 62 - protein, 392 aa, Pantoea vagans
  REGION 1..392: note = MISC_FEATURE - Vag MFS transporter, WP_048785139.1
MKSLLTRKRRINPVFLAFMAASFMIGVAGALQAPTLSLFLTREVQARPLWVGLFFTVNAIAGIVVSMLVAKRSDSRGDRRTLILFCCAMAFCNALLFAFTRHYLTLITLGVLLSALASVSMPQIFALAREYADQSAREAVMFSSVMRAQLSLAWVIGPPLSFALALNFGFVTLFLVAAALFLVCILLIKFTLPSVPRAEPLMRSGGMPLSGWRDRDVRLLFIASVTMWTCNTMYIIDMPLYISVTLGLPEKLAGLLMGTAAGLEIPVMLLAGHYAKRVGKRNLMLIAVAAGVLFYAGLAMFASQTALMALQLFNAVFIGIIAGIGMLWFQDLMPGRPGAATTMFTNSISTGMILAGVIQGTLSERFGHIAVYWLALGLAVAAFAMSARVKNV

SEQ ID NO: 63 - protein, 393 aa, Yersinia frederiksenii
  REGION 1..393: note = MISC_FEATURE - Fred MFS transporter, WP_087817556.1
MKSALTFSRRINPVFLAFFVVAFLSGIAGALQAPTLSLFLSTEVKVRPLWVGLFYTVNAIAGITVSFVLAKRSDLRGDRRKLILVCYLMAVGNCLLFAFNRDYLTLITAGVLLAAVANTAMPQIFALAREYADNSAREVVMFSSIMRAQLSLAWVIGPPLSFMLALNYGFTLMFCIAAGIFVLSALVVWFILPSVQRAEPVMDAPTVAQGSLFADKDVLLLFIASMLMWTCNTMYIIDMPLYITASLGLPERLAGLLMGTAAGLEIPIMLLAGYSVRRFGKRKIMLFAVLAGVLFYTGLVLFKFKSALMLLQIFNAIFIGIVAGIGMLYFQDLMPGRAGAATTLFTNSISTGVILAGVLQGVLTETWGHNSVYVMAMILAILSLIICARVREA

SEQ ID NO: 64 - protein, 398 aa, Serratia marcescens
  REGION 1..398: note = MISC_FEATURE - Marc MFS transporter, WP_060448169.1
MQRLSRLSLRINPIFAAFLLIAFLSGIAGALLTPTLSLFLTTEVKVRPLWVGLFYTANAVAGIVVSFLLAKRSDTRGDRRRLILLCCLMAVGNCLLFAFNRDYLTLITAGVLMSAVANTAMPQIFALAREYADSEAREVVMFSSVMRAQLSLAWVIGPPLSFALALNYGFTVMFLIAAVTFAVCVLLVGFMLPSVPRAAENEGLQGGVSAPIAPASAWRNRDVRLLFIASMLMWTCNTLYIIDMPLYITADLGLPEGLAGVLMGTAAGLEIPAMLLAGYYVKRFGKRNMMLLAVVAGVLFYLGLTVLESKPALIALQLLNAVFIGIVAGIGMLYFQDLMPGRPGAATTLFTNSISTGVILAGVLQGALVENLGHGSVYWMAALLALAALGMSAKVREV

SEQ ID NO: 65 - protein, 387 aa, Rouxiella badensis
  REGION 1..387: note = MISC_FEATURE - Bad MFS transporter, WP_017489914.1
MSSRRLSIIFATFLLVSFLTGIAGALQAPTLSLFLTNEVKVRPLWVGLFYTVNALGGIVISFLLANYSDKKGDRRKLLFFCTLMAIGNSLIFAYSRDYLVLISVGVLLAAIGNASMPQLFALAREYADRSAHEVVMFSSMMRATLSLAWVLGPPISFTLALNYGFTLMYLCAAGVFIFSALMVWFFLPSVGRIEQPVDKVVVHVSAWKNRDVRLLFFASLLMWTCNIMYIIDMPLYITSDLGLPEGLAGLLMGAAAGLEIPVMLIAGYLVKRTGKRRLMLCAAVFGILFYLGLVLFQFKAALMILQLFNAIFIGIIAGIGMLYFQDLMPGRAGSATTLFTNSISTGAILAGVIQGTIVQNFGHYQVYWMALALAVGALVLMTRVKNV

SEQ ID NO: 66 - protein, 394 aa, Rosenbergiella nectarea
  REGION 1..394: note = MISC_FEATURE - Nec MFS transporter, WP_092672081.1
MQSFTPPAPKGGNPVFMMFMLVTFFVSIAGALQAPTLSLYLSQELAAKPFMVGLFFTINAVTGIIISFILAKRSDRKGDRRRLLMFCCAMAIANALMFAFVRQYVVLITLGLILSALTSVVMPQLFALAREYADRTGREVVMFSSVMRTQMSLAWVIGPPISFALALNYGFITLYLVAAALFLLSLILIKTTLPSVPRLYPAEDLAKSAASGWKRTDVRFLFAASVLMWVCNLMYIIDMPLYISKSLGMPESFAGVLMGTAAGLEIPVMLLAGYLAKRVGKRPLVIVAAVCGLAFYPAMLVFHQQTGLLIIQLLNAVFIGIVAGLVMLWFQDLMPGKAGAATTLFTNSVSTGMIFAGLCQGLLSDLLGHQAIYVLATVLMVIALLLLLRVKEQA

SEQ ID NO: 67 - protein, 393 aa, Yersinia bercovieri
  REGION 1..393: note = MISC_FEATURE - YberC MFS transporter
MKSALTFSRRINPVFLAFFVVAFLSGIAGALQAPTLSLFLSTEVKVRPLWVGLFYTVNAIAGITVSFILAKRSDSRGDRRKLIMVCYLMAVGNCLLFAFNRDYLTLITAGVLLASVANTAMPQIFALAREYADSSAREVVMFSSIMRAQLSLAWVIGPPLSFMLALNYGFTLMFSIAAGIFVLSALVVWFILPSVPRAEPVVDAPVVVQGSLFADKNVLLLFIASMLMWTCNTMYIIDMPLYITASLGLPERLAGLLMGTAAGLEIPIMLLAGYSVRYFGKRKIMLFAVLAGVLFYTGLVLFKFKTALMLLQIFNAIFIGIVAGIGMLYFQDLMPGRAGAATTLFTNSISTGVILAGVLQGGLTETWGHDSVYVMAMVLSILALIICARVREA

SEQ ID NO: 68 - DNA, 107 bp, Escherichia coli
  misc_feature 1..107: note = Plac, lac operon promoter
  source 1..107: mol_type = other DNA
tgtgagttagctcactcattaggcaccccaggctttacactttatgcttccggctcgtatgttgtgtggaattgtgagcggataacaatttcacacaggaaacagct

SEQ ID NO: 69 - DNA, 291 bp, synthetic construct
  misc_feature 1..291: note = PgatY_70UTR, variant of E. coli promoter for gatYZABCD; tagatose-1,6-bisP aldolase
  source 1..291: mol_type = other DNA
cggcaacctatgcctgatgcgacgctgaagcgtcttatcatgcctacatagcactgccacgtatgtttacaccgcatccggcataaaaacacgcgcactttgctacggcttccctatcgggaggccgtttttttgcctttcactcctcgaataattttcatattgtcgtttttgtgatcgttatctcgatatttaaaaacaaataatttcattatattttgtgcctacaagcatcgtggaggtccgtgactttcacgcatacaacaaacattaaccaaggaggaaacagct

SEQ ID NO: 70 - DNA, 300 bp, synthetic construct
  misc_feature 1..300: note = PglpF, E. Coli promoter sequence of glpFKX operon
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaggaggaaacagct

SEQ ID NO: 71 - DNA, 300 bp, synthetic construct
  misc_feature 1..300: note = PglpF_SD1, variant of PglpF E. Coli promoter sequence of glpFKX operon
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaattcgaaacagct

SEQ ID NO: 72 - DNA, 300 bp, synthetic construct
  misc_feature 1..300: note = PglpF_SD10, variant of PglpF E. Coli promoter sequence of glpFKX operon
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactgagaaacagct

SEQ ID NO: 73 - DNA, 300 bp, synthetic construct
  misc_feature 1..300: note = PglpF_SD2, variant of PglpF E. Coli promoter sequence of glpFKX operon
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagcgcaaaacagct

SEQ ID NO: 74 - DNA, 300 bp, synthetic construct
  misc_feature 1..300: note = PglpF_SD3 variant of PglpF E. Coli promoter sequence of glpFKX operon
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagaacaaaacagct

SEQ ID NO: 75 - DNA, 300 bp, synthetic construct
  misc_feature 1..300: note = PglpF_SD4, variant of PglpF E. Coli promoter sequence of glpFKX operon
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactaggaaacagct

SEQ ID NO: 76 - DNA, 300 bp, synthetic construct
  misc_feature 1..300: note = PglpF_SD5, variant of PglpF E. Coli promoter sequence of glpFKX operon
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaccgagaaacagct

SEQ ID NO: 77 - DNA, 300 bp, synthetic construct
  misc_feature 1..300: note = PglpF_SD6, variant of PglpF E. Coli promoter sequence of glpFKX operon
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagctaaacagct

SEQ ID NO: 78 - DNA, 300 bp, synthetic construct
  misc_feature 1..300: note = PglpF_SD7, variant of PglpF E. Coli promoter sequence of glpFKX operon
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagcaaaacagct

SEQ ID NO: 79 - DNA, 300 bp, synthetic construct
  misc_feature 1..300: note = PglpF_SD8, variant of PglpF E. Coli promoter sequence of glpFKX operon
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagaaaaacagct

SEQ ID NO: 80 - DNA, 300 bp, synthetic construct
  misc_feature 1..300: note = PglpF_SD9, variant of PglpF E. Coli promoter sequence of glpFKX operon
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaaggaaaaacagct

SEQ ID NO: 81 - DNA, 107 bp, synthetic construct
  misc_feature 1..107: note = Plac_16UTR, variant of E. coli lac operon promoter
  source 1..107: mol_type = other DNA
tgtgagttagctcactcattaggcaccccaggctttacactttatgcttccggctcgtatgttgtgtggaattgtgagcggataacaatttcaaggaggaaacagct

SEQ ID NO: 82 - DNA, 203 bp, synthetic construct
  misc_feature 1..203: note = PmglB_70UTR, variant of E. coli promoter for mglBAC galactose/methyl-galactosidade transporter
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaggaggaaacagct

SEQ ID NO: 83 - DNA, 203 bp, synthetic construct
  misc_feature 1..203: note = PmglB_70UTR_SD4, variant of E. coli promoter for mglBAC; galactose/methyl-galactosidade transporter
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactaggaaacagct

SEQ ID NO: 84 - DNA, 76 bp, synthetic construct
  misc_feature 1..76: note = CP6 promotor sequence
  source 1..76: mol_type = other DNA
catgtgggagtttattcttgacacagatatttccggatgatataataactgagtactgttcacacaggaaacagct

SEQ ID NO: 85 - DNA, 269 bp, Escherichia coli
  misc_feature 1..269: note = PosmY, promotor for osmY-ytjA; perplasmic chaperone
  source 1..269: mol_type = other DNA
caaaattgtgatctatatttaacaaagtgatgacatttctgacggcgttaaataccgttcaatgcgtagatatcagtatctaaagccgtcgattgtcattctaccgatattaataactgattcagaggctgtaatggtcgttattcatcactcatcgcttttgtgatggcgaccattgacttctgtagagggtgaagtctctccctattcagcaatgcaacctcgtgttgccaggctcaaattacgagcaaaccaaggaggaaacagct

SEQ ID NO: 86 - protein, 483 aa, Avibacterium gallinarum
  REGION 1..483: note = MISC_FEATURE - SacC_Agal, glycoside hydrolase family 32 protein, WP_103853210.1
MIIFNEGKYKSLYAAEQGELEKIAQTVAQDQDFRPVYHLAPPTGLLNDPNGLIFDGEKYHLFYQWYPFDALHGMKHWQHFITQDFKQFSQADLLVPCELYESHGCYSGGAVKIGDQIAVFYTGNTRRPSDNQRVPYQNLAIFSKDGKLLSKRPLIEQAPQGYTEHVRDPKPFLTKDGKIRFICGAQRENLTGTALVFEMDNLADTPRLLGELALPAFDNQGVFMWECPDLSQMGDKSLFIWSPQGKARELEQYQNNYHAVYALGELADRQFHAEQIAELDQGFDFYAPQTFSGTQTMLLGWVGLPDLSYPTDLYKWHSMLSMPRQLRLQDGKIYQQPIENIYKNLTALQSITVEKEAEIADLDRAYLKFDANAQPFSLKFFNNAQNQRLILSYDGEMLCLDRSQTEQTDSMKSFGDKRYCRIEDLRQVEIFFDRSVAEIFLNQGEKAMTSRFFICARENQLCTDKPLTLQVGYPKKIEVDYTK

SEQ ID NO: 87 - protein, 548 aa, Arthrobacter globiformis
  REGION 1..548: note = MISC_FEATURE - Bff, beta-fructofuranosidase protein, BAD18121.1
MERTCITVRAIVRFHIEQRQTIVNKQRTKRGILTAALSIGALGATLISGPAVAATDAAPGFPQPTEHTQKAYSPTDNFTSRWTRADAKQLKAMSDPDAGSRENSMPTEYTMPTVSQDFPDMSNEKVWVWDTWPLIDENANQYSVNGQEIIFSLVADRKLGFDERHQYARIGYFYRPAGIPADERPEDGGWTYGGQVFDEGVTGKIFEDQSFTHQTQWSGSARVSKNGEIKLFFTDVAFYRDKDGQDVKPYDSRIALSVGHVHSNKKGVKLTGFNKVKELLQADGKNYQNAAQNSYYNFRDPFTFVDPAHPGETYMVFEGNSAMDRDEAKCTAEDLGYREGETNGETVEQVNNSGATYQIGNVGLARAKNKALTEWEFLPPILSANCVTDQTERPQIYMQDGKYYLFTISHRSTFATGIDGPEGVYGFVGNGIRSDYQPLNRGSGLALGSPTNLNFAAGTPFAPDYNQHPGQFQAYSHYVMPGGLVQSFIDTIGTKDNFVRGGTLGPTVKLNIKGDSATVDYNYGDNGLGGWADIPANRELKNSKAVAK

SEQ ID NO: 88 - protein, 505 aa, Klebsiella pneumoniae
  REGION 1..505: note = MISC_FEATURE - ScrY, sucrose porin, GeneBank ID CAA40657.1
MYKKRKLAILIALLTGTAAAHGQTDLNSIEARLAALEKRLQDAETRASTAESRAASAEQKVQQLTQQQQQTQATTQQVARRTTQLEEKAERPGGFEFHGYARSGVIMNDSAASTKSGAYMTPAGETGGAIGRLGNQADTYVEMNLEHKQTLDNGATTRFKVMVADGQTTYNDWTASSSDLNVRQAFVELGNLPTFEGPFKGSTLWAGKRFDRDNFDIHWIDSDVVFLAGTGGGIYDVKWNDSLRSNFSLYGRNFGDIADSSNSVQNYIVSMNNFAGPVQMMVSGMRAKDNDDRQDANGNLVKGDAANTGVHALLGLHNESFYGLRDGTSKTALLYGHGLGAEVKGIGSDGALRPGANTWRFASYGTTPLSDRWFIAPAVLAQSSKDRYVDGDSYQWATLNLRLIQEVTQNFALAWEGSYQYMDLQPEGYNDRHAVNGSFYKLTFAPTFKVGSIGDFFSRPEIRFYTSWMDWSKKLDNYANDDALGSNGFKSGGEWSFGMQMETWF

SEQ ID NO: 89 - protein, 456 aa, Klebsiella pneumoniae
  REGION 1..456: note = MISC_FEATURE - ScrA, sucrose-specific enzyme II, GeneBank ID: CAA40658.1
MDFEQISRSLLPLLGGKENIASAAHCATRLRLVLVDDALADQQAIGKIDGVKGCFRNAGQMQIIFGTGVVNKVYAAFIQAAGISESSKSEAADLAAKKLNPFQRIARLLSNIFVPIIPAIVASGLLMGLLGMVKTYGWVDPSNALYIMLDMCSSAAFIILPILIGFTAAREFGGNPYLGATLGGILTHPALTNAWGVAAGFHTMNFFGIEVAMIGYQGTVFPVLLAVWFMSMVEKRLRRVIPDALDLILTPFLTVIISGFIALLLIGPAGRALGDGISFILSTLISHAGWLAGLLFGGLYSVIVITGIHHSFHAIEAGLLGNPSIGVNFLLPIWAMANVAQGGACFAVWFKTKDAKIKAITLPSAFSAMLGITEAAIFGINLRFVKPFIAALVGGAAGGAWVVSMHVYMTAVGLTAIPGMAIVQASSLLNYIIGMAIAFAVAFALSLTLKYKTDAE

SEQ ID NO: 90 - protein, 466 aa, Salmonella enterica
  REGION 1..466: note = MISC_FEATURE - ScrB, beta-fructofuranosidase, GeneBank ID: CAA47974.1
MSLPSRLPAILQAVMQGQPRALADSHYPRWHHAPVTGLMNDPNGFIEFAGRYHLFYQWNPLACDHTFKCWAHWSSIDLLHWQHEPIALMPDEEYDRNGCYSGSAVDNNGTLTLCYTGNVKFAEGGRTAWQCLATENADGTFRKIGPVLPLPEGYTGHVRDPKVWRHEDLWYMVLGAQDRQKRGKVLLFSSADLHQWTSMGEIAGHGINGLDDVGYMWECPDLFPLGDQHILICCPQGIAREEECYLNTYPAVWMAGEFDYAAGAFRHGELHELDAGFEFYAPQTMLTSDGRRLLVGWMGVPEGEEMLQPTLNNGWIHQMTCLRELEFINGQLYQRPLRELSALRGEANGWSGNALPLAPMEIDLQTRGGDMLSLDFGGVLTLECDASGLRLARRSLASDEMHYRYWRGNVRSLRVFIDQSSVEIFINGGEGVMSSRYFPACSGQLTFSGITPDAFCYWPLRTCMVE

SEQ ID NO: 91 - protein, 334 aa, Salmonella enterica
  REGION 1..334: note = MISC_FEATURE - ScrR, sucrose repressor, GeneBank ID: CAA47975.1
MKTKRVTIKDIAEQAGVSKATASLVLNGRGKELRVAQETRERVLSIARKHHYQPSIHARSLRNNRSHTIGLVVPEITNHGFAVFAHELEMLCREAGVQLLISCTDENPGQESVVVNNMIARQVDGMIVASCMHNDADYLKLSQQLPVVLFDRCPNESALPLVMTDSITPTAELISRIAPQHSDEFWFLGGQARLSPSRDRLTGFTQGLAQAGIALRPEWVINGNYHPSSGYEMFAALCARLGRPPKALFTAACGLLEGVLRYMSQHHLLDSDIHLTSFDDHYLYDSLSLRIDTVQQDNRQLAWHCYDLISQLIEGDTPETLQRYLPATLQFRHQ

SEQ ID NO: 92 - DNA, 730 bp, Escherichia coli
  misc_feature 1..730: note = glpR coding sequence
  source 1..730: mol_type = other DNA
atgaaacaaacacaacgtcacaacggtattatcgaactggttaaacagcagggttatgtcagtaccgaagagctggtagagcatttctccgtcagcccgcagactattcgccgcgacctcaatgagctggcggagcaaaacctgatcctggccatcatggcggtgcggcgctgccttccagttcggttaacacgccgtggcacgatcgcaaggccacccagaccgaagaaaaagagcgcatcgcccgcaaagtggcggagcaaatccccaatggctcgacgctgtttatcgatatcggcaccacgccggaagcggtagcgcacgcactgctcaatcacagcaatttgcgcattgtcaccaacaatctcaacgttgctaacacgttgatggtaaaagaagattttcgcatcattctcgccggtggcgaattacgcagccgcgatggcgggatcattggcgaagcgacgctcgattttatctcccagttccgccttgatttcggcattctggggataagcggcatcgatagcgacggctcgctgctggagttcgattaccacgaagttcgcaccaaacgcgccattattgagaactcgcgccacgttatgctggttgtcgatcactcgaaatttggccgtaacgcgatggtcaatatgggcagcatcagcatggtagatgccgtctacaccgacgccccgccgccagtaagcgtgatgcaggtgctgacgga

SEQ ID NO: 93 - DNA, 203 bp, synthetic construct
  misc_feature 1..203: note = PmglB_70UTR_SD8 promoter sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagaaaaacagct

SEQ ID NO: 94 - DNA, 203 bp, synthetic construct
  misc_feature 1..203: note = PmglB_70UTR_SD10 promoter sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactgagaaacagct

SEQ ID NO: 95 - DNA, 203 bp, synthetic construct
  misc_feature 1..203: note = PmglB-54UTR promoter sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaacaaaaaccggagatacc

SEQ ID NO: 96 - DNA, 141 bp, synthetic construct
  misc_feature 1..141: note = Plac 70_UTR promoter sequence
  source 1..141: mol_type = other DNA
tgtgagttagctcactcattaggcaccccaggctttacactttatgcttccggctcgtatgttgtgtggaatgcctacaagcatcgtggaggtccgtgactttcacgcatacaacaaacattaaccaaggaggaaacagct

SEQ ID NO: 97 - DNA, 203 bp, synthetic construct
  misc_feature 1..203: note = PmglB_70UTR_SD9 promoter sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaaggaaaaacagct

SEQ ID NO: 98 - DNA, 203 bp, synthetic construct
  misc_feature 1..203: note = PmglB_70UTR_SD5 promoter sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaccgagaaacagct

SEQ ID NO: 99 - DNA, 203 bp, synthetic construct
  misc_feature 1..203: note = PmglB_70UTR_SD7 promoter sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagcaaaacagct

SEQ ID NO: 100 - DNA, 189 bp, synthetic construct
  misc_feature 1..189: note = PglpA_70UTR promoter sequence
  source 1..189: mol_type = other DNA
gaaaacattcataaattaaatgtgaattgccgcacacattattaaataagatttacaaaatgttcaaaatgacgcatgaaatcacgtttcactttcgaattatgagcgaatatgcgcgatgcctacaagcatcgtggaggtccgtgactttcacgcatacaacaaacattaaccaaggaggaaacagct

SEQ ID NO: 101 - DNA, 239 bp, synthetic construct
  misc_feature 1..239: note = PglpT_70UTR promoter sequence
  source 1..239: mol_type = other DNA
ccatttagccatagtaaaaacatgaattgtttgatttcgcgcatattcgctcataattcgaaagtgaaacgtgatttcatgcgtcattttgaacattttgtaaatcttatttaataatgtgtgcggcaattcacatttaatttatgaatgttttcttaacatcgcggcatgcctacaagcatcgtggaggtccgtgactttcacgcatacaacaaacattaaccaaggaggaaacagct

SEQ ID NO: 102 - DNA, 350 bp, synthetic construct
  misc_feature 1..350: note = PmglB_16UTR promoter sequence
  source 1..350: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgttttaacgttgtaacccgtatgtaacagtgaataatcacttttgccgaggtaacagcgtcataacaacaattaaagccgttttctggagcgttaccgggcatggaagaacgaattttaaaaagtgagcttcggcgttcagtaacacttcattaactctactgccccgccgagcatttatctcaagcactaccctgcataagcaaggaggaaacagct

SEQ ID NO: 103 - DNA, 189 bp, synthetic construct; identical to SEQ ID NO: 100
  misc_feature 1..189: note = PglpA_16UTR promoter sequence
  source 1..189: mol_type = other DNA

(19) United States
(12) Patent Application Publication
(10) Pub. No.: US 2026/0015636 A1
(43) Pub. Date: Jan. 15, 2026

(54) ENHANCING FORMATION OF HUMAN MILK OLIGOSACCHARIDES (HMOS) BY MODIFYING LACTOSE IMPORT IN THE CELL

(71) Applicant: DSM IP Assets B.V., Heerlen (DK)

(72) Inventor: Manos PAPADAKIS, Hørsholm (DK)

(21) Appl. No.: 19/191,943

(22) Filed: Apr. 28, 2025

Related U.S. Application Data
(63) Continuation of application No. 18/561,258, filed on Nov. 15, 2023, now Pat. No. 12,286,658.

(30) Foreign Application Priority Data
May 17, 2021 (DK) PA202170249

(51) Int. Cl. C12P 19/00; C12N 9/10; C12N 15/52

(52) U.S. Cl. CPC C12P 19/00; C12N 9/1051; C12N 15/52; C12Y 204/01038; C12Y 204/01122; C12Y 204/01147

(57) ABSTRACT

This invention relates to a method of producing one or more human milk oligosaccharides (HMOs), in particular LNT and/or LNnT, in a genetically engineered cell comprising an enhanced oligosaccharide transport capability. The genetically modified cell comprises a series of genetic modification which enable the production of one or more HMO(s), and a series of genetic modification that enhances the transport of lactose and produced HMO(s).

### CROSS-REFERENCE TO RELATED APPLICATIONS

This application is a continuation of U.S. patent application Ser. No. 18/513,315, filed Nov. 17, 2023, as a national stage entry of International Application Number PCT/EP2022/063311, filed on May 17, 2022, which claims priority to Denmark Application No. PA202170249, filed May 17, 2021, the entire contents of all of which are hereby incorporated by reference in their entirety.

### SEQUENCE LISTING

This instant application contains a Sequence Listing which has been submitted in a ASCII text file via Patent Center and is hereby incorporated by reference in its entirety. Said text file, created on Apr. 28, 2025, is named 032991-8038 Sequence Listing.txt, and is 277,123 bytes in size.

### FIELD

This invention relates to a method of producing one or more human milk oligosaccharides (HMOs), in particular LNT and/or LNnT, in a genetically engineered cell comprising an enhanced oligosaccharide transport capability. The genetically modified cell comprises a series of genetic modification that enables the production of one or more HMOs, and a series of genetic modification that enhances the transport of lactose and produced HMO(s).

### BACKGROUND

Human milk oligosaccharides (HMO(s)) have become of great interest in the last decade, due to the discovery of their important functionality in human development. Besides their prebiotic properties, HMO(s) have been linked to additional positive effects, expanding their field of application. The health benefits of HMO(s) have enabled their approval for use in food, such as infant formulas and food, and for consumer health products.

To date, the structures of at least 115 HMO(s) have been determined, and considerably more are probably present in human milk.

Due to the limited availability of HMO(s), an effective commercial, i.e., large scale production, is highly desirable. The manufacturing of large-scale quantities, as well as qualities, required for food and medical applications, through chemical synthesis, has yet to be provided. Furthermore, chemical synthetic routes to HMO(s) involve several noxious chemicals, which impose a contamination risk to the final product.

To bypass the drawbacks associated with chemical synthesis of HMO(s), several enzymatic methods and fermentative approaches have been developed. Fermentation based processes have been developed for several HMO(s), such as 2′-fucosyllactose, 3-fucosyllactose, Iacto-N-tetraose, lacto-N-neotetraose, 3′-sialyllactose and 6′-sialyllactose. Fermentation based processes typically utilize genetically engineered bacterial strains, such as recombinant Escherichia coli (E. coli), or yeast, such as Saccharomyces cerevisiae (S. cerevisiae).

Biosynthetic production of HMO(s) in engineered bacterial strains is a valuable, cost-efficient and large-scale applicable solution for HMO manufacturing. It relies on genetically engineered bacteria with modified metabolic engineering, such as modified sugar nucleotide synthesis pathway(s) and are constructed so as to express the glycosyltransferases needed for the synthesis of the desired oligosaccharides and often take advantage of the bacteria's pool of nucleotide sugars as HMO precursors.

Recent developments in biotechnological production of HMO(s) have made it possible to overcome certain inherent limitations of bacterial expression systems. For example, HMO-producing bacterial cells may be genetically engineered to increase the limited intracellular pool of nucleotide sugars in the bacteria (WO2012112777), to improve activity of enzymes involved in the HMO production (WO2016040531), or to facilitate the import of HMO precursors and secretion of synthesized HMO(s) into the extracellular media (WO2010142305, WO2017042382). Further, expression of genes of interest in recombinant cells may be regulated by using particular promoter sequences or other gene expression regulators, e.g., as recently described in WO2019123324.

One way to optimize the production of HMOs is to modify sugar transport, where especially the import of the HMO precursor lactose could have a favorable effect on the HMO production, due to its role as a starting point for HMO biosynthesis. This has been done by overexpression of the E. coli, lactose permease LacY, which represents one of the most intensively characterized solute transporters (Guan and Kaback, Annu Rev Biophys Biomol Struct. 2006; 35:67-91).

WO2014018596 suggests enhancing the production of 2′-FL, LDFT, LNFP-I or LNDFH-I through a modified lactose catabolism and overexpression of the E. coli lactose permease, LacY, wherein the expression of LacY is driven by a laclq promoter, an inducible synthetic promoter with much higher levels of transcription than the wild-type promoter (Penumetcha, et al., BIOS, 81(1):7-15 (2010)). This enhances the intracellular pool of lactose and phosphor-nucleotides, leading to an enhanced HMO production.

WO2017042382 combines inactivating the endogenous beta-galactosidase gene and overexpressing or deleting a native or heterologous sugar efflux transporter and having a functional lactose permease protein, in order to produce one or more HMOs. Specific examples show that deletion of oligosaccharide exporters can increase LNT formation.

EP2927316 discloses 2′FL production from a host with a native LacY gene under control of the Ptet promoter and the YberC transporter expressed from a plasmid. It also discloses that overexpression of LacY in the presence of lactose can cause lactose-induced stress of the cell and therefore the cell has been genetically modified to be able to totally ferment an oligosaccharide of interest without the exogenous addition of lactose.

Herein, a method is for the first time disclosed for producing one or more HMOs, that comprises a combination of enhancing the expression of a lactose permease gene, such as an endogenous lactose permease gene, with the expression of a heterologous non-LacY sugar transporter, which surprisingly facilitates an optimized transportation equilibrium leading to an effectively enhanced production of one or more HMOs, while also reducing the amount of side product formation.

### SUMMARY

The present disclosure relates to a genetically engineered cell and a method for producing one or more HMO(s) in particular LNT and/or LNnT, wherein the method comprises a suitably genetically engineered cell in which the lactose uptake system is upregulated in combination with an expression of a second sugar transporter, thus promoting the import/export of lactose and/or oligosaccharides in the genetically engineered cell to advance the HMO production. The identification of key steps in the cellular pathways that regulate the biosynthesis of HMOs has enabled the modification of said pathways to enhance the cellular production of HMOs by regulating the cellular equilibrium between metabolites and products. In particular, maintenance of the favourable lactose/HMO equilibrium has been achieved, wherein overexpression of the lactose permease, LacY, in combination with another sugar transporter from the MFS transporter family, enhances the level of overall LNT and/or LNnT produced by the cell, as well as the transport of particular HMOs into the fermentation media. In general, the invention promotes a more sustainable manufacturing process, wherein the conversion from carbon source to HMO product in fermentation is done at a higher overall yield.

In its broadest sense, the present disclosure thus relates to a method for producing one or more HMO(s), in particular LNT and/or LNnT, which comprises providing a genetically engineered cell, wherein the cell overexpresses one or more lactose permease(s), expresses a heterologous MFS transporter selected from the group consisting of Vag, Nec, Fred, Marc, YberC, Bad and a functional homologue of any one of Vag, Nec, Fred, Marc, YberC or Bad, having an amino acid sequence which is 80% identical to the amino acid sequence of any one of Vag, Nec, Fred, Marc, YberC or Bad, and wherein the cell further expresses one or more glycosyltransferases selected from the group consisting of β-1,3-GlcNAc-transferase,β-1,3-Gal-transferase and β-1,4-gal-transferase and optionally a sucrose utilisation system. The method comprises culturing said cell in a suitable medium containing lactose and harvesting the one or more HMO(s) produced.

In a preferred embodiment, the genetically engineered cell of the present invention overexpresses one or more native lactose permease(s).

The amino acid sequence of the one or more lactose permease(s) is selected from the group consisting of SEQ ID NOs 1, 2, 3, 4, 5, 6, 7, 8, 9, 10, 11, 12, 13, 14, 15, 16 [LacY variants] and an amino acid sequence which is at least 70% identical to any one of the SEQ ID NOs 1, 2, 3, 4, 5, 6, 7, 8, 9, 10, 11, 12, 13, 14, 15, 16 [LacY variants] and which is a functional homologue.

In a presently preferred embodiment, the one or more overexpressed lactose permease(s) is identical to SEQ ID NO: 1 [LacY K-12] or at least 70% identical to SEQ ID NO: 1 [LacY K-12]. The genetically engineered cell may comprise at least one nucleic acid sequence encoding a lactose permease, or it may comprise more than one nucleic acid sequence encoding one or more lactose permeases(s). The nucleic acid sequence encoding the lactose permease is preferably a native gene to the genetically engineered cell, it may be encoded by a recombinant nucleic acid sequence.

The heterologous MFS transporter protein is selected from the group of SEQ ID NOs: 62 [Vag], 63 [Fred], 64 [Marc], 65 [Bad], 66 [Nec], 67 [YberC], and a functional homologue of any one of SEQ ID NOs: 62 [Vag], 63 [Fred], 64 [Marc], 65 [Bad], 66 [Nec], 67 [YberC], having an amino acid sequence which is at least 70% identical to any one of SEQ ID NOs: 62 [Vag], 63 [Fred], 64 [Marc], 65 [Bad], 66 [Nec], 67 [YberC]. In one or more exemplary embodiment(s), the MFS transporter is Nec, YberC and/or Vag. The genetically engineered cell may comprise at least one nucleic acid sequence encoding one or more heterologous MFS transporter(s), according to the invention. It may comprise more than one nucleic acid sequence encoding one or more heterologous MFS transporter(s). The nucleic acid sequence encoding the heterologous MFS transporter(s) may be encoded by a synthetic or recombinant nucleic acid sequence.

The genetically engineered cell may comprise one nucleic acid sequence encoding one or more glycosyltransferase(s) selected from β-1,3-GlcNAc-transferase, β-1,3-Gal-transferase and β-1,4-gal-transferase according to the invention. It may comprise more than one nucleic acid sequence encoding one or more glycosyltransferase(s). The nucleic acid sequence encoding the one or more glycosyltransferase(s) may be encoded by a synthetic or recombinant nucleic acid sequence.

In one or more exemplary embodiment(s) of the present disclosure, the β-1,3-Gal-transferase or β-1,4-gal-transferase is selected from the group consisting of CvB3galT and GalTK, or GalT, respectively and a functional homologue of any one of CvB3galT, GalTK or GalT, having an amino acid sequence which is at least 70% identical to any one of SEQ ID NOs: 17 [CvB3galT], 18 [GalTK] and 19 [GalT].

In one or more further exemplary embodiment(s) of the present disclosure, the β-1,3-GlcNAc-transferase is selected from the group consisting of LgtA, PmnagT, HD0466 and a functional homologue of any one of LgtA, PmnagT or HD0466, having an amino acid sequence which is at least 70% identical to any one of SEQ ID NO: 20 [LgtA], 21 [PmnagT] or 22 [HD0466].

In one or more exemplary embodiment(s), the genetically engineered cell comprises one or more heterologous nucleic acid sequence encoding one or more heterologous polypeptide(s) which when expressed constitute a sucrose utilisation system that enables utilization of sucrose as sole carbon and energy source of said genetically engineered cell. In one or more preferred exemplary embodiment(s), the genetically engineered cell comprises expresses a PTS-dependent sucrose utilization system, further comprising the scrYA and scrBR operons. In another preferred embodiment, the genetically engineered cell expresses a polypeptide capable of hydrolysing sucrose into glucose and fructose, preferably a single polypeptide.

One way to enhance the HMO production is also to modify the biosynthesis of the activated sugar nucleotides, which are used in the biosynthesis of the HMOs. Thus, the genetically engineered cell may comprise at least one nucleic acid sequence encoding one or more heterologous polypeptides involved in the biosynthesis of activated sugars according to the present disclosure. In one or more exemplary embodiment(s) the genetically engineered cell expresses one or more polypeptides involved in the biosynthesis of activated sugar nucleotides, such as one or more polypeptide(s) selected from the group consisting of Pgm [SEQ ID NO: 43 or 44], GalU [SEQ ID NO: 45 or 46], GalE [SEQ ID NO: 47 or 48], GlmM [SEQ ID NO: 49 or 50], GlmU [SEQ ID NO: 51 or 52], GlmS [SEQ ID NO: 33 or 54], and a functional homologue thereof having an amino acid sequence which is at least 70% identical to any one of SEQ ID NOs: 43, 44, 45, 46, 47, 48, 49, 50, 51, 52, 53 or 54.

Genetic expression of proteins in the genetically engineered cell according to the invention is regulated by regulatory elements. Thus, any one or more nucleic acid sequence(s) according to the invention may be regulated by one or more regulatory element(s). In one or more exemplary embodiment(s) the regulatory element comprises one or more promoter sequence (swith a nucleic acid sequence as identified in Table 8, preferably a promoter sequence selected from the group consisting of SEQ ID NO: 70 (PglpF) or SEQ ID NO: 68 (Plac) or SEQ ID NO: 69 (PgatY_70UTR), SEQ ID NO: 82 (PmglB_UTR70) or SEQ ID NO: 100 (PglpA_70UTR) or SEQ ID NO: 101 (PglpT_70UTR) or cp6 (SEQ ID NO: 84) or Posmy (SEQ ID NO: 85) or variants of these.

In a presently exemplified embodiment, in the genetically engineered cell of the present invention, the one or more lactose permease(s) is regulated by the promoter sequence PglpF.

In another presently exemplified embodiment, in the genetically engineered cell of the present invention, the one or more heterologous MFS transporter(s) is regulated by the promoter sequence Plac and/or PglpF.

Thus, in a presently preferred embodiment, in the genetically engineered cell of the present invention, the one or more lactose permease(s) is native and is regulated by the promoter sequence PglpF and the one or more heterologous MFS transporter(s) is selected from the group consisting of Nec, YberC and/or Vag and is regulated by the promoter sequence Plac and/or PglpF.

The regulatory element may further comprise an endogenous, heterologous, synthetic and/or optimized Shine-Dalgarno sequence.

The nucleic acid sequence(s) comprising the elements of the present disclosure, may be integrated into the genome of the genetically engineered cell. Alternatively, the one or more nucleic acid sequence(s) may be expressed from a plasmid, such as a one or more low, medium and/or high copy number plasmid(s).

In one or more exemplary embodiment(s), the genetically engineered cell is a bacterial or yeast cell, such as an Escherichia coli cell, such as preferably a strain derived from Escherichia coli K-12.

The invention also relates to the use of the method and/or the genetically engineered cell in the production and/or manufacturing of one or more HMOs, such as LNT and/or LNnT.

### DETAILED DESCRIPTION

The present disclosure relates to a genetically engineered cell and a method for producing one or more HMO(s), wherein the lactose uptake system of the cell is upregulated in combination with an expression of a second sugar transporter, thus promoting the import/export of oligosaccharides in the genetically engineered cell to advance the HMO production of in particular LNT and LNnT.

In the present disclosure, the cellular production of LNT and/or LNnT is enhanced by regulating the cellular equilibrium between metabolites and products. In particular, maintenance of the favourable lactose/HMO equilibrium has been achieved, wherein overexpression of the lactose permease, LacY, in combination with another sugar transporter from the MFS transporter family, in particular selected from the group consisting of Vag, Nec, Fred, Marc, YberC and Bad, enhances the level of over-all HMO produced by the cell, as well as the level of HMO transported into the fermentation media, resulting in a higher overall yield of HMO(s) produced by the engineered cell, in particular LNT and/or LNnT.

Regulation of the biosynthesis of HMOs has enabled the modification of cellular pathways that may enhance the cellular production of HMOs by regulating the cellular equilibrium between metabolites and products. By the term “biosynthesis” is meant a synthesis of one or more HMOs which is conducted by and/or in a genetically engineered cell. The cellular equilibrium between metabolites and products is an essential feature to maintain cellular viability, where accumulation of either the metabolites, such as phosphates and/or product precursors e.g., lactose, can lead to cellular stress which reduces the efficiency of the HMO production. On the other hand, cellular accumulation of product, such as one or more HMOs, may also lead to cellular stress responses, which may also hamper the efficiency of the HMO production. Thus, engineering of the cellular pathways, tailoring each point of the biosynthesis of HMOs in combination with a tailored import/export system, so that cellular accumulation of metabolites and/or HMOs is reduced, while enhancing the export of the produced HMO(s), is highly advantageous in large scale production of HMOs.

In the present disclosure, the maintenance of a favourable cellular equilibrium between metabolites and products has been achieved through overexpression of the lactose permease, LacY, in combination with another sugar transporter, such as but not limited to the MFS transporters YberC, Nec or Vag, which enhances the overall level of produced LNT or LNnT and in some cases affects biomass formation, as described in examples 1-3.

In specific, the present disclosure relates to a genetically engineered cell, which overexpresses one or more lactose permease(s) in combination with a heterologous MFS transporter protein, selected from the group consisting of Vag, Nec, Fred, Marc, YberC, Bad and a functional homologue of any one of Vag, Nec, Fred, Marc, YberC or Bad, having an amino acid sequence which is 70% identical to the amino acid sequence of any one of Vag, Nec, Fred, Marc, YberC or Bad, and which further expresses one or more glycosyltransferase(s). In a preferred embodiment the genetically engineered cell expresses two or more glycosyltransferase(s), wherein the glycosyltransferases are a β-1,3-GlcNAc-transferase and a β-1,3-Gal-transferase, or a β-1,3-GlcNAc-transferase and a β-1,4-gal-transferase, or a β-1,3-GlcNAc-transferase and a β-1,3-Gal-transferase and a β-1,4-Gal-transferase.

In a preferred embodiment, the present disclosure relates to a genetically engineered cell, which overexpresses one or more native lactose permease(s) in combination with a heterologous MFS transporter protein, selected from the group consisting of Vag, Nec, Fred, Marc, YberC, Bad and a functional homologue of any one of Vag, Nec, Fred, Marc, YberC or Bad, having an amino acid sequence which is 70% identical to the amino acid sequence of any one of Vag, Nec, Fred, Marc, YberC or Bad, and which further expresses one or more glycosyltransferase(s). In a preferred embodiment the genetically engineered cell expresses two or more glycosyltransferase(s), wherein the glycosyltransferases are a β-1,3-GlcNAc-transferase and a β-1,3-Gal-transferase, or a β-1,3-GlcNAc-transferase and a β-1,4-gal-transferase, or a β-1,3-GlcNAc-transferase and a β-1,3-Gal-transferase and a β-1,4-Gal-transferase.

### A Method for Producing One or More Human Milk Oligosaccharide(s)

The present disclosure provides to a method for producing one more human milk oligosaccharides (HMOs), in particular LNT and/or LNnT, that comprises a combination of enhancing the expression of a lactose permease gene with the expression of a heterologous non-LacY sugar transporter, which surprisingly facilitates an enhanced production of the one or more HMOs, while also reducing the amount of side product formation. Preferably, said lactose permease gene is endogenous to the host cell.

This is illustrated in e.g., example 1 and 2, wherein an additional copy of LacY is inserted into a strain that already expresses the heterologous transporter YberC or Nec or Vag and which enhances the production of the LNT in example 1, and LNnT and pLNnH in example 2, and which at the same time reduces the by-product formation, such as a reduction in LNT-II and pLNH2 production. It is also evident from example 3 that the HMO titer in fermentation reaches a higher level, upon overexpression of LacY, compared to the normal expression level of LacY. The lower by-product formation and the higher titer are features that are essential in a large-scale production setting, where a lower by-product formation may result in a simpler post-fermentation purification and where the higher titer may result in a higher production yield, thus reducing the HMO production cost.

### Human Milk Oligosaccharide (HMO)

In the context of the disclosure, the term “oligosaccharide” means a saccharide polymer containing a number of monosaccharide units. In some embodiments, preferred oligosaccharides are saccharide polymers consisting of three, four, five or six monosaccharide units, i.e., trisaccharides, tetrasaccharides, penta saccharides or hexasaccharides. Preferable oligosaccharides of the disclosure are human milk oligosaccharides (HMOs).

The term “human milk oligosaccharide” or “HMO” in the present context means a complex carbohydrate found in human breast milk. The HMOs have a core structure comprising a lactose unit at the reducing end that can be elongated by one or more beta-N-acetyl-lactosaminyl and/or one or more beta-lacto-N-biosyl units, and this core structure can be substituted by an alpha-L-fucopyranosyl and/or an alpha-N-acetyl-neuraminyl (sialyl) moiety.

In the context of the present disclosure, lactose is not regarded as an HMO species.

The non-acidic (or neutral) HMOs are devoid of a sialyl residue, and the acidic HMOs have at least one sialyl residue in their structure. The method of the present disclosure is particularly designed to enhance the production and over-all yield of both neutral and acidic HMOs.

The non-acidic (or neutral) HMOs can be fucosylated or non-fucosylated. Examples of such neutral non-fucosylated HMOs include lacto-N-triose II (LNT-II) lacto-N-tetraose (LNT), lacto-N-neotetraose (LNnT), lacto-N-neohexaose (LNnH), para-lacto-N-neohexaose (pLNnH), para-lacto-N-hexaose (pLNH) and lacto-N-hexaose (LNH). Examples of neutral fucosylated HMOs include 2′-fucosyllactose (2′-FL), lacto-N-fucopentaose I (LNFP-I), lacto-N-difucohexaose I (LNDFH-I), 3-fucosyllactose (3-FL), difucosyllactose (DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (F-pLNnH II) and fucosyl-lacto-N-neohexaose (FLNnH). Examples of acidic HMOs include 3′-sialyllactose (3′-SL), 6′-sialyllactose (6′-SL), 3-fucosyl-3′-sialyllactose (FSL), 3′-O-sialyllacto-N-tetraose a (LST a), fucosyl-LST a (FLST a), 6′-O-sialyllacto-N-tetraose b (LST b), fucosyl-LST b (FLST b), 6′-O-sialyllacto-N-neotetraose (LST c), fucosyl-LST c (FLST c), 3′-O-sialyllacto-N-neotetraose (LST d), fucosyl-LST d (FLST d), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DSLNT).

In one or more preferred embodiment(s), the one or more produced HMO is selected from the group consisting of LNT-II, pLNnH, LNT and LNnT, LNFP-I, LNFP-II, LNFP-III, LNFP-V, LNFP-VI, LNDFH-I, LNDFH-II, LNDFH-III, 2′-FL, DFL, 3-FL, LST-a, 3′-SL, 6′-SL, LST-b, LST-c, FSL, FLST-a, DSLNT, LNnH and LNH.

Preferably, the HMO produced by the herein disclosed method and/or genetically engineered cell is an HMO that comprises an LNT-II core, such as LNT and/or LNnT, pLNnH and/or LNFP-I.

Even more preferably, the produced HMO is LNT and/or LNnT.

In one or more exemplary embodiment(s) the produced HMO is LNT. In one or more further exemplary embodiment(s) the produced HMO is LNnT.

LNT, LNnT and LNFP-I comprise an LNT-II core and may be synthesized in a suitably genetically engineered cell by selecting a series of glycosyltransferases, that have the capability of attaching specific saccharides, such as galactose (Gal) or N-Acetylglucosamine (GlcNAc) to a lactose backbone.

### A Genetically Engineered Cell

A genetically engineered cell of the present disclosure is a host cell that has been genetically engineered so that it is for HMO production.

In particular, the present disclosure relates to a engineered cell, which overexpresses one or more lactose permease(s) in combination with a heterologous MFS transporter protein, selected from the group consisting of Vag, Nec, Fred, Marc, YberC, Bad and a functional homologue of any one of Vag, Nec, Fred, Marc, YberC or Bad, having an amino acid sequence which is 70% identical to the amino acid sequence of any one of Vag, Nec, Fred, Marc, YberC or Bad, and which further expresses one or more glycosyltransferase(s). The one or more lactose permease(s) can be native to the host cell.

A genetically engineered cell can in general contain one or more genes that are not present in the native (not genetically engineered) form of the cell. Techniques for introducing exogenous nucleic acid molecules/sequences and/or inserting exogenous nucleic acid molecules/sequences (recombinant, heterologous) into a cell's hereditary information for inserting, deleting or altering the nucleic acid sequence of a cell's genetic information are known to the skilled artisan.

A genetically engineered cell according to the present disclosure can contain one or more genes that are present in the native form of the cell, wherein said genes are modified and re-introduced into the microbial cell by artificial means.

The term “genetically engineered cell” also encompasses a cell that contains a nucleic acid molecule being endogenous to the cell that has been modified without removing the nucleic acid molecule from the cell. Such modifications include those obtained by gene replacement, site-specific mutations, and related techniques.

A genetically engineered cell of the invention can be provided using standard methods of the art e.g., those described in the manuals by Sambrook et al., Wilson & Walker, “Maniatis et al., and Ausubel et al.

The genetically engineered cell may be any cell useful for HMO production including mammalian cell lines. Preferably, the host cell is a unicellular microorganism of eucaryotic or prokaryotic origin. Appropriate microbial cells that may function as a host cell include yeast cells, bacterial cells, archaebacterial cells, algae cells, and fungal cells.

In one or more exemplary embodiment(s), the genetically engineered cell is a prokaryotic cell. In one or more preferred exemplary embodiment(s), the genetically engineered cell is a bacterial cell.

The genetically engineered cell of the present disclosure may be eubacteria (gram-positive or gram-negative) or archaebacteria, as long as they allow genetic manipulation for insertion of a gene of interest and can be cultivated on a manufacturing scale. Preferably, the host cell has the property to allow cultivation to high cell densities.

In embodiments of the invention the genetically engineered cell is a bacterial or yeast cell. In one preferred embodiment, the genetically engineered cell is preferably a prokaryotic cell, such as a bacterial cell.

Non-limiting examples of bacterial cells that are suitable for use in the present disclosure are selected from recipient organisms that do not naturally contain any of the mentioned MFS transporters Vag, Nec, Fred, Marc, YberC and Bad. These include, but are not limited to, E. coli K-12 strains (such as EMG2, MG1655, W3110, W3350, C600, and DH5a), which is the strain background for the majority of genetic engineering work done in E. coli, ATCC 8739 (E. coli C), ATCC 11303 (E. coli B), BL21 (see New England Biolabs catalog, 2007-2008) and derivatives of these strains.

In a presently preferred embodiment, the genetically engineered cell of the invention is an Escherichia coli cell. In a particularly preferred embodiment, the genetically engineered cell of the invention is an E. coli cell derived from the E. coli K-12 strain or DE3 strain.

In one or more exemplary embodiments, the genetically engineered cell is selected from the group consisting of E. coli, C. glutamicum, L. lactis, B. subtilis, S. lividans.

In one or more exemplary embodiments, the genetically engineered cell is selected from the group consisting of B. subtilis, and E. coli.

In one or more exemplary embodiments, the genetically engineered cell is selected from the group consisting of E. coli, C. glutamicum, L. lactis, B. subtilis, S. lividans.

In one or more exemplary embodiments, the genetically engineered cell is B. subtilis.

In one or more exemplary embodiments, the genetically engineered cell is Corynebacterium glutamicum.

In one or more exemplary embodiments, the genetically engineered cell is E. coli.

Non-limiting examples of fungal host cells that are suitable for recombinant industrial production of a HMO product could be yeast cells, such as Komagataella phaffii, Kluyveromyces lactis, Yarrowia lipolytica, Pichia pastoris, and Saccharomyces cerevisiae or filamentous fungi such as Aspergillus sp, Fusarium sp or Trichoderma sp, exemplary species are A. niger, A. nidulans, A. oryzae, F. solani, F. graminearum and T. reesei.

In one or more exemplary embodiments, the genetically engineered cell is selected from the group consisting of Yarrowia lipolytica, Pichia pastoris, and Saccharomyces cerevisiae.

In one or more exemplary embodiments, the genetically engineered cell is S. cerevisiae or P. pastoris.

In one or more exemplary embodiments, the genetically engineered cell is Pichia pastoris.

### Expression in the Genetically Engineered Cell

In relation to the invention, the term “expression” may refer to expression of a gene or a nucleic acid sequence which results in the production of a protein, or it may relate to expression of a protein i.e., it may relate directly to the production of a protein. In specific, the term “expression” may refer to the expression of a lactose permease gene, which results in the expression of a lactose permease protein, or to the expression of a lactose permease protein, as well as to the expression of an MFS transporter protein gene or an MFS transporter protein, respectively.

The use of the term “enhanced expression” is in the present context used interchangeably with the term “overexpression” e.g., of a lactose permease protein, and refers to an elevated level of a protein being expressed compared to the normal level of protein expression of a native protein in said cell. Enhanced expression of a protein or gene can be achieved in multiple ways, and may comprise, modification of the gene copy number, controlling the expression of any copy of a gene at the transcriptional or the translational level, e.g. by substituting the native promoter with a strong promoter, deleting of regulatory elements that repress the expression of a gene, or introduction of an episomal element, such as a plasmid, that bears and expresses the coding sequence of the gene of interest.

Increasing the gene copy number and/or the expression of one or more genes encoding the enzymes and/or transporter proteins that are directly or indirectly involved in the HMO biosynthetic pathways may be advantageous, as described herein and exemplified in examples 1-3.

In one or more exemplary embodiment(s), expression is controlled by increasing the copy number of the gene or nucleic acid sequence encoding a protein of interest.

In one or more exemplary embodiment(s), expression is controlled by using a strong promoter sequence to enhance expression of the gene or nucleic acid sequence encoding a protein of interest.

### Overexpressing One or More Lactose Permease(s)

The present disclosure relates to a genetically engineered cell, as well as to a method comprising said genetically engineered cell, which overexpresses one or more lactose permease(s). In a preferred embodiment, the present disclosure relates to a genetically engineered cell, as well as to a method comprising said genetically engineered cell, which overexpresses one or more native lactose permease(s).

The overexpression of one or more lactose permease(s) according to the present disclosure results in an enhanced production of one or more HMO(s), in particular LNT and/or LNnT, while in some cases also in a reduction of the amount of side products in the production, as described in examples 1-3.

By “overexpression” is in the present context meant that the expression level of protein is higher than what is obtained naturally by the cell of the invention. Thus, introduction of, and expression of a heterologous gene is not considered “overexpression” in relation to the invention. Overexpression may be determined by transcriptional or translational analysis, of for instance, quantitative determination of mRNA levels or protein levels in any of the methods known to the person skilled in the art, such as but not limited to, quantitative PCR or mass spectrometry. In the present disclosure, overexpression is preferably determined by experimental observation of an effect which may be related to an enhanced expression of one or more lactose permease(s), such as the experimental data provided in examples 1-3 and FIGS. 1-5.

### A Lactose Permease Gene

In one or more embodiment(s) of the present invention, a genetically engineered cell comprises one or more lactose permease genes which is/are overexpressed. A lactose permease gene can be a lacYgene.

The genetically engineered cell according to the invention may comprise least one, such as at least two, three, four, five, six, seven, eight, nine or ten nucleic acid sequence(s) encoding a lactose permease.

In one or more preferred exemplary embodiment(s) the genetically engineered cell comprises more than one nucleic acid sequence encoding one or more lactose permeases(s).

In one or more exemplary embodiment(s), the genetically engineered cell comprises one or more additional lactose permease(s), such as one or more heterologous lactose permease.

In one or more further exemplary embodiment(s) the one or more lactose permease(s) is/are encoded by a heterologous and/or recombinant nucleic acid sequence.

In one or more preferred exemplary embodiment(s) the nucleic acid sequence(s) encoding the one or more lactose permease(s) is a native gene to the genetically engineered cell.

### Native Lactose Permease Gene

In one or more exemplified embodiments, a genetically engineered cell of the present disclosure comprises a lactose permease gene native to the host cell which is overexpressed.

A native lactose permease of the present disclosure is a gene which is found endogenously in the host cell of the invention. E.g., a native lactose permease gene can be selected from the lacY gene of E coli K-12 MG1655 of SEQ ID NO: 2, encoding the lactose permease LacY of SEQ ID NO: 1, in an E coli K-12 MG1655 derived strain. Thus, the lacY gene native to E. coli BL-21(DE3) overexpressed in an E. coli K-12 MG1655 derived strain would not be native to the E. coli K-12 MG1655 derived strain, given that the gene encoding the E. coli BL-21(DE3) LacY is non-identical to the gene encoding the E. coli K-12 MG1655 LacY.

### LacY

In one embodiment, the one or more lactose permease(s) may be any one or more of the amino acid sequences selected from the group consisting of SEQ ID NOs 1, 2, 3, 4, 5, 6, 7, 8, 9, 10, 11, 12, 13, 14, 15, 16 [LacY variants] and functional homologues thereof, having an amino acid sequence which is at least 70% identical, such as at least 71, 72, 73, 74, 75, 76, 77, 78, 79, 80, 81, 82, 83, 84, 85, 86, 87, 88, 89, 90, 91, 92, 93, 94, 95, 96, 97, 98, 99 or 99.9% identical, to any one of the SEQ ID NOs 1, 2, 3, 4, 5, 6, 7, 8, 9, 10, 11, 12, 13, 14, 15 or 16 [LacY variants].

In one or more preferred exemplary embodiment(s), the one or more lactose permease(s) is/are identical to SEQ ID NO: 1.

In one or more embodiment(s), the lactose permease is a native lactose permease.

### MFS Transporter Protein

The genetically engineered cell of the present disclosure comprises at least one nucleic acid sequence encoding one or more heterologous MFS transporter protein(s). The genetically engineered cell of the present disclosure thus expresses a heterologous MFS transporter protein.

The transporters of the Major Facilitator Superfamily (MFS) facilitate the transport of molecules, such as but not limited to oligosaccharides, across the cellular membranes.

By the term “Major Facilitator Superfamily (MFS)” is meant a large and exceptionally diverse family of the secondary active transporter class, which members are responsible for transporting a range of different substrates, including sugars, drugs, hydrophobic molecules, peptides, organic ions, etc.

The term “MFS transporter” in the present context means a protein that facilitates transport of an oligosaccharide, preferably, an HMO, through or across the cell membrane, preferably of an HMO/oligosaccharide synthesized by the genetically engineered cell as described herein from the cell cytosol to the cell medium. Additionally, or alternatively, the MFS transporter may also facilitate efflux of molecules that are not considered HMO or oligosaccharides, such as lactose, glucose, cell metabolites and/or toxins. In a preferred embodiment the MFS transporter is capable of exposting LNT and/or LNnT from the cell cytosol to the cell medium.

In the context of the present invention the lactose permease is not considered to be a heterologous MFS transporter.

In example 1-3, it is shown how introduction of selected heterologous genes encoding sugar efflux transporter proteins in the genetic background of LacY expressing strains, or LacY overexpressing strains, can inverse the order of abundance of the HMO precursors and/or side products of the cellular HMO production and thus facilitate an improved production of the HMO(s) by the genetically modified cell.

In one or more exemplary embodiment(s), the MFS transporter is selected from the group consisting of Bad, Nec, YberC, Fred, Vag and Marc.

The genetically engineered cell of the present disclosure thus in one or more exemplary embodiment(s) expresses a heterologous MFS transporter protein selected from the group consisting of Vag, Nec, Fred, Marc, YberC, Bad and a functional homologue of any one of Vag, Nec, Fred, Marc, YberC or Bad having an amino acid sequence which is 70% identical to the amino acid sequence of any one of SEQ ID NOs: 62 [Vag], 63 [Fred], 64 [Marc], 65 [Bad], 66 [Nec] or 67 [YberC].

### Bad

The MFS transporter protein identified herein as “Bad protein” or “Bad transporter” or “Bad”, interchangeably, has the amino acid sequence of SEQ ID NO: 65; The amino acid sequence identified herein as SEQ ID NO: 65 is an amino acid sequence that has 100% identity with the amino acid sequence having the GenBank accession ID WP_017489914.1.

In one or more exemplary embodiment(s), the MFS transporter, expressed according to the present disclosure is Bad. The genetically engineered cell of the present disclosure thus in one or more exemplary embodiment(s) expresses a heterologous MFS transporter protein that is Bad.

In one or more embodiment(s) of the invention, the genetically engineered cell expresses the heterologous MFS transporter protein bad or a functional homologue thereof, having an amino acid sequence which is at least 80%, such as at least 90%, such as at least 95%, such as at least 99% identical to the amino acid sequence of bad [SEQ ID NO: 65].

In one or more embodiment(s) the expression of bad is regulated by a regulatory element comprising the Plac promoter element of SEQ ID NO: 68.

In one embodiment of the invention, the genetically engineered cell, which overexpresses one or more native lactose permease(s) in combination with the heterologous MFS transporter protein, Bad of SEQ ID NO: 65, or a functional homologue thereof, having an amino acid sequence which is 70% identical to the amino acid sequence of SEQ ID NO: 65, and which further expresses one or more glycosyltransferase(s).

### Nec

The MFS transporter protein identified herein as “Nec protein” or “Nec transporter” or “Nec”, interchangeably, has the amino acid sequence of SEQ ID NO: 66; The amino acid sequence identified herein as SEQ ID NO: 66 is an amino acid sequence that has 100% identity with the amino acid sequence having the GenBank accession ID WP_092672081.1.

In one or more exemplary embodiment(s), the MFS transporter, expressed according to the present disclosure is Nec. The genetically engineered cell of the present disclosure thus in one or more exemplary embodiment(s) expresses a heterologous MFS transporter protein that is Nec.

In one or more embodiment(s) of the invention, the genetically engineered cell expresses the heterologous MFS transporter protein Nec or a functional homologue thereof, having an amino acid sequence which is at least 80%, such as at least 90%, such as at least 95%, such as at least 99% identical to the amino acid sequence of Nec [SEQ ID NO: 66].

In one or more embodiment(s) the expression of Nec is regulated by a regulatory element comprising the Plac promoter element of SEQ ID NO: 68.

In one embodiment of the invention, the genetically engineered cell, which overexpresses one or more native lactose permease(s) in combination with the heterologous MFS transporter protein, nec of SEQ ID NO: 66, or a functional homologue thereof, having an amino acid sequence which is 70% identical to the amino acid sequence of SEQ ID NO: 66, and which further expresses one or more glycosyltransferase(s).

### YberC

The MFS transporter protein identified herein as “YberC protein” or “YberC transporter” or “YberC”, interchangeably, has the amino acid sequence of SEQ ID NO: 67; The amino acid sequence identified herein as SEQ ID NO: 67 is an amino acid sequence that has 100% identity with the amino acid sequence having the GenBank accession ID EEQ08298.1.

In one or more exemplary embodiment(s), the MFS transporter, expressed according to the present disclosure is YberC. The genetically engineered cell of the present disclosure thus in one or more exemplary embodiment(s) expresses a heterologous MFS transporter protein that is YberC.

In one or more embodiment(s) of the invention, the genetically engineered cell expresses the heterologous MFS transporter protein YberC or a functional homologue thereof, having an amino acid sequence which is at least 80%, such as at least 90%, such as at least 95%, such as at least 99% identical to the amino acid sequence of YberC [SEQ ID NO: 67].

In one or more embodiment(s) the expression of YberC is regulated by a regulatory element comprising the Plac promoter element of SEQ ID NO: 68.

In one embodiment of the invention, the genetically engineered cell, which overexpresses one or more native lactose permease(s) in combination with the heterologous MFS transporter protein, YberC of SEQ ID NO: 67, or a functional homologue thereof, having an amino acid sequence which is 70% identical to the amino acid sequence of SEQ ID NO: 67, and which further expresses one or more glycosyltransferase(s).

### Fred

The MFS transporter protein identified herein as “Fred protein” or “Fred transporter” or “Fred”, interchangeably, has the amino acid sequence of SEQ ID NO: 63; The amino acid sequence identified herein as SEQ ID NO: 63 is an amino acid sequence that has 100% identity with the amino acid sequence having the GenBank accession ID WP_087817556.1.

In one or more exemplary embodiment(s), the MFS transporter, expressed according to the present disclosure is Fred. The genetically engineered cell of the present disclosure thus in one or more exemplary embodiment(s) expresses a heterologous MFS transporter protein that is Fred.

In one or more embodiment(s) of the invention, the genetically engineered cell expresses the heterologous MFS transporter protein fred or a functional homologue thereof, having an amino acid sequence which is at least 80%, such as at least 90%, such as at least 95%, such as at least 99% identical to the amino acid sequence of fred [SEQ ID NO: 63].

In one or more embodiment(s) the expression of fred is regulated by a regulatory element comprising the Plac promoter element of SEQ ID NO: 68.

In one embodiment of the invention, the genetically engineered cell, which overexpresses one or more native lactose permease(s) in combination with the heterologous MFS transporter protein, Fred of SEQ ID NO: 63, or a functional homologue thereof, having an amino acid sequence which is 70% identical to the amino acid sequence of SEQ ID NO: 63, and which further expresses one or more glycosyltransferase(s).

### Vag

The MFS transporter protein identified herein as “Vag protein” or “Vag transporter” or “Vag”, interchangeably, has the amino acid sequence of SEQ ID NO: 62; The amino acid sequence identified herein as SEQ ID NO: 62 is an amino acid sequence that has 100% identity with the amino acid sequence having the GenBank accession ID WP_048785139.1.

In one or more exemplary embodiment(s), the MFS transporter, expressed according to the present disclosure is Vag. The genetically engineered cell of the present disclosure thus in one or more exemplary embodiment(s) expresses a heterologous MFS transporter protein that is Vag.

In one or more embodiment(s) of the invention, the genetically engineered cell expresses the heterologous MFS transporter protein vag or a functional homologue thereof, having an amino acid sequence which is at least 80%, such as at least 90%, such as at least 95%, such as at least 99% identical to the amino acid sequence of vag [SEQ ID NO: 62].

In one or more embodiment(s) the expression of vag is regulated by a regulatory element comprising the Plac promoter element of SEQ ID NO: 68.

In one embodiment of the invention, the genetically engineered cell, which overexpresses one or more native lactose permease(s) in combination with the heterologous MFS transporter protein, vag of SEQ ID NO: 62, or a functional homologue thereof, having an amino acid sequence which is 70% identical to the amino acid sequence of SEQ ID NO: 62, and which further expresses one or more glycosyltransferase(s).

### Marc

The MFS transporter protein identified herein as “Marc protein” or “Marc transporter” or “Marc”, interchangeably, has the amino acid sequence of SEQ ID NO: 64; The amino acid sequence identified herein as SEQ ID NO: 64 is an amino acid sequence that has 100% identity with the amino acid sequence having the GenBank accession WP_060448169.1.

In one or more exemplary embodiment(s), the MFS transporter, expressed according to the present disclosure is Marc. The genetically engineered cell of the present disclosure thus in one or more exemplary embodiment(s) expresses a heterologous MFS transporter protein that is Marc.

In one or more embodiment(s) of the invention, the genetically engineered cell expresses the heterologous MFS transporter protein marc or a functional homologue thereof, having an amino acid sequence which is at least 80%, such as at least 90%, such as at least 95%, such as at least 99% identical to the amino acid sequence of marc [SEQ ID NO: 64].

In one or more embodiment(s) the expression of marc is regulated by a regulatory element comprising the Plac promoter element of SEQ ID NO: 68.

In one embodiment of the invention, the genetically engineered cell, which overexpresses one or more native lactose permease(s) in combination with the heterologous MFS transporter protein, marc of SEQ ID NO: 64, or a functional homologue thereof, having an amino acid sequence which is 70% identical to the amino acid sequence of SEQ ID NO: 64, and which further expresses one or more glycosyltransferase(s).

The genetically engineered cell of the present disclosure thus in one or more exemplary embodiment(s) expresses a heterologous MFS transporter protein which is either Vag, Nec, Fred, Marc, YberC or Bad. In one or more further exemplary embodiment(s), the genetically engineered cell of the present disclosure expresses more than one heterologous MFS transporter protein selected from the group consisting of SEQ ID NOs: 62 [Vag], 63 [Fred], 64 [Marc], 65 [Bad], 66 [Nec], and 67 [YberC].

In one or more exemplary embodiment(s), the genetically engineered cell of the present disclosure expresses a functional homologue of Vag, Nec, Fred, Marc, YberC and/or Bad having an amino acid sequence which is at least 70%, 80%, 85%, 90%, 95% or at least 99% identical to any one of SEQ ID NOs: 62, 63, 64, 65, 66 or 67.

In a presently preferred embodiment, the MFS transporter expressed is Nec, Vag and/or YberC.

In an especially preferred embodiment, the MFS transporter expressed is YberC.

In an especially preferred embodiment, the MFS transporter expressed is Nec.

In an especially preferred embodiment, the MFS transporter expressed is Vag.

### Heterologous Expression

In the present disclosure, the term heterologous means that a protein is experimentally put into a cell that does not normally make (i.e., express) that protein. Thus, heterologous refers to the fact that often the transferred protein was initially cloned from or derived from a different cell type or a different species from the recipient. The protein itself may not necessarily be transferred, but instead the ‘correctly edited’ genetic material coding for the protein (the complementary DNA or cDNA) can be added to the recipient cell. The genetic material that is transferred typically must be within a format that encourages the recipient cell to express the cDNA as a protein (i.e., it is put in an expression vector). Methods for transferring foreign genetic material into a recipient cell include transfection and transduction. The choice of recipient cell type is often based on an experimental need to examine the protein's function in detail, and the most prevalent recipients, known as heterologous expression systems, are chosen usually because they are easy to transfer DNA into or because they allow for a simpler assessment of the protein's function.

### Glycosyltransferase

The genetically engineered cell of the present disclosure for use according to the present disclosure further expresses one or more glycosyltransferase(s). The nucleic acid sequence encoding the one or more expressed glycosyltransferase(s) may be integrated into the genome (by chromosomal integration) of the genetically engineered cell, or alternatively, it may be comprised in a plasmid and expressed as plasmid-borne, as described in the present disclosure.

If two or more glycosyltransferases are needed for the genetically engineered cell to be suitable of producing an HMO, e.g., LNT or LNnT, two or more heterologous nucleic acids encoding different enzymes with glycosyltransferase activity may be integrated into a nucleic acid construct or they may be provided as individual nucleic acid sequences, which may be integrated in the genome and/or expressed from a plasmid.

In one exemplary embodiment, a β-1,3-N-acetylglucosaminyltransferase (a first heterologous nucleic acid sequence encoding a first glycosyltransferase) is introduced into the host cell in combination with a β-1,3-galactosyltransferase (a second heterologous nucleic acid sequence encoding a second glycosyltransferase) for the production of LNT, where the first and second heterologous nucleic acid can independently from each other be integrated chromosomally or be expressed from a plasmid or they can be combined into a nucleic acid construct, optionally comprised in a nucleic acid construct also comprising the additional features of the present disclosure. Thus, in one or more exemplary embodiment(s), the genetically engineered cell of the invention comprises at least one nucleic acid sequence encoding one or more heterologous glycosyltransferase(s).

In another exemplary embodiment, a β-1,3-N-acetylglucosaminyltransferase (a first heterologous nucleic acid sequence encoding a first glycosyltransferase) is introduced into the host cell in combination with a β-1,4-Galactosyltransferase (a second heterologous nucleic acid sequence encoding a second glycosyltransferase) for the production of LNnT, where the first and second heterologous nucleic acid can independently from each other be integrated chromosomally or be expressed from a plasmid or they can be combined into a nucleic acid construct, optionally comprised in a nucleic acid construct also comprising the additional features of the present disclosure. Thus, in one or more exemplary embodiment(s), the genetically engineered cell of the invention comprises at least one nucleic acid sequence encoding one or more heterologous glycosyltransferase(s).

In one or more exemplary embodiment(s), both the first and second heterologous nucleic acid encoding one or more glycosyltransferase(s) is/are stably integrated into the chromosome of the genetically engineered cell; in another preferred embodiment, the first and second heterologous nucleic acid encoding one or more glycosyltransferases are integrated independently of the nucleic acid sequence encoding the native lactose permease and/or the MFS transporter, according to the invention. In one or more further exemplary embodiment(s), the first and second heterologous nucleic acids encoding one or more glycosyltransferases are integrated into a nucleic acid construct. In one or more further exemplary embodiment(s), at least one of the heterologous nucleic acid sequence(s) encoding the glycosyltransferase(s) are plasmid-borne.

A glycosyltransferase of the present disclosure is selected from the group of enzymes having the activity of an α-1,2-fucosyltransferase, α-1,3-fucosyltransferase, α-1,3/4-fucosyltransferase, α-1,4-fucosyltransferase α-2,3-sialyltransferase, α-2,6-sialyltransferase, β-1,3-N-acetylglucosaminyltransferase, β-1,6-N-acetylglucosaminyltransferase, β-1,3-galactosyltransferase and β-1,4-galactosyltransferase.

In a preferred embodiment the glycosyl transferase(s) are selected from the group consisting of β-1,3-GlcNAc-transferase,β-1,3-Gal-transferase and β-1,4-gal-transferase.

Table 1 shows some non-limiting embodiments of proteins having glycosyltransferase activity which can be encoded by the heterologous genes comprised in the production cell.

TABLE 1

| | GenBank or | | |
|---|---|---|---|
| | NCBI Reference | | |
| Gene | Sequence ID | Description | HMO example |
| IgtA_Nm | WP_002248149.1 | β-1,3-N- | LNT-II, LNT, LNnT, LNFP-I, |
| | | acetylglucosaminyl- | LNFP-II, LNFP-III, LNFP-V, |
| | | transferase | LNFP-VI, LNDFH-I, LNDFH-II, |
| | | | LNDFH-III, pLNH, F-pLNH I, |
| | | | pLNnH |
| IgtA_Nm_MC58 | AAF42258.1 | β-1,3-N- | LNT-II, LNT, LNnT, LNFP-I, |
| | | acetylglucosaminyl- | LNFP-II, LNFP-III, LNFP-V, |
| | | transferase | LNFP-VI, LNDFH-I, LNDFH-II, |
| | | | LNDFH-III, pLNH, F-pLNH I, |
| | | | pLNnH |
| IgtA_Hd | AAN05638.1 | β-1,3-N- | LNT-II, LNT, LNnT, LNFP-I, |
| | | acetylglucosaminyl- | LNFP-II, LNFP-III, LNFP-V, |
| | | transferase | LNFP-VI, LNDFH-I, LNDFH-II, |
| | | | LNDFH-III, pLNH, F-pLNH I, |
| | | | pLNnH |
| IgtA_Ng_PID2 | AAK70338.1 | β-1,3-N- | LNT-II, LNT, LNnT, LNFP-I, |
| | | acetylglucosaminyl- | LNFP-II, LNFP-III, LNFP-V, |
| | | transferase | LNFP-VI, LNDFH-I, LNDFH-II, |
| | | | LNDFH-III, pLNH, F-pLNH I, |
| | | | pLNnH |
| IgtA_Ng_NCCP | ACF31229.1 | β-1,3-N- | LNT-II, LNT, LNnT, LNFP-I, |
| 11945 | | acetylglucosaminyl- | LNFP-II, LNFP-III, LNFP-V, |
| | | transferase | LNFP-VI, LNDFH-I, LNDFH-II, |
| | | | LNDFH-III, pLNH, F-pLNH I, |
| | | | pLNnH |
| IgtA_Past | AAK02595.1 | β-1,3-N- | LNT-II, LNT, LNnT, LNFP-I, |
| | | acetylglucosaminyl- | LNFP-II, LNFP-III, LNFP-V, |
| | | transferase | LNFP-VI, LNDFH-I, LNDFH-II, |
| | | | LNDFH-III, pLNH, F-pLNH I, |
| | | | pLNnH |
| IgtA_Nc | EEZ72046.1 | β-1,3-N- | LNT-II, LNT, LNnT, LNFP-I, |
| | | acetylglucosaminyl- | LNFP-II, LNFP-III, LNFP-V, |
| | | transferase | LNFP-VI, LNDFH-I, LNDFH-II, |
| | | | LNDFH-III, pLNH, F-pLNH I, |
| | | | pLNnH |
| IgtA_Nm_87255 | ELK60643.1 | β-1,3-N- | LNT-II, LNT, LNnT, LNFP-I, |
| | | acetylglucosaminyl- | LNFP-II, LNFP-III, LNFP-V, |
| | | transferase | LNFP-VI, LNDFH-I, LNDFH-II, |
| | | | LNDFH-III, pLNH, F-pLNH I, |
| | | | pLNnH |
| galT_Hp/ | NP_207619.1 | β-1,4- | LNnT, LNFP-III, LNFP-VI, |
| HP0826 | | galactosyltransferase | pLNH I, F-pLNH I, pLNnH |
| galT_Nm/ | AAF42257.1 | β-1,4- | LNnT, LNFP-III, LNFP-VI, |
| IgtB | | galactosyltransferase | pLNH I, F-pLNH I, pLNnH |
| wbgO | WP_000582563.1 | β-1,3- | LNT, LNFP-I, LNFP-II, LNFP-V, |
| | | galactosyltransferase | LNDFH-I, LNDFH-II, pLNH, F- |
| | | | pLNH I |
| cpsIBJ | AB050723.1 | β-1,3- | LNT, LNFP-I, LNFP-II, LNFP-V, |
| | | galactosyltransferase | LNDFH-I, LNDFH-II, pLNH, F- |
| | | | pLNH I |
| jhp0563 | AEZ55696.1 | β-1,3- | LNT, LNFP-I, LNFP-II, LNFP-V, |
| | | galactosyltransferase | LNDFH-I, LNDFH-II, pLNH, F- |
| | | | pLNH I |
| galTK | SEQ ID NO: 18 | β-1,3- | LNT, LNFP-I, LNFP-II, LNFP-V, |
| | | galactosyltransferase | LNDFH-I, LNDFH-II, pLNH, F- |
| | | | pLNH I |
| futC | SEQ ID NO: 23 | α-1,2-fucosyl- | 2′-FL, DFL, LNFP-I, LNDFH-I |
| | | transferase | |
| FucT2_HpUA802 | AAC99764.1 | α-1,2-fucosyl- | 2′-FL, DFL, LNFP-I, LNDFH-I |
| | | transferase | |
| FucT2_EcO126t | ABE98421.1 | α-1,2-fucosyl- | 2′-FL, DFL, LNFP-I, LNDFH-I |
| | | transferase | |
| FucT2_Hm12198 | CBG40460.1 | α-1,2-fucosyl- | 2′-FL, DFL, LNFP-I, LNDFH-I |
| | | transferase | |
| FucT2_Pm9515 | ABM71599.1 | α-1,2-fucosyl- | 2′-FL, DFL, LNFP-I, LNDFH-I |
| | | transferase | |
| FucT2_HpF57 | BAJ59215.1 | α-1,2-fucosyl- | 2′-FL, DFL, LNFP-I, LNDFH-I |
| | | transferase | |
| FucT6_3_Bf | CAH09151.1 | α-1,3-fucosyl- | 2′-FL, 3-FL, DFL, LNFP-I, |
| | | transferase | LNFP-III, LNFP-V, LNFP-VI, |
| | | | LNDFH-II, F-pLNH I |
| FucT7_3_Bf | CAH09495.1 | α-1,3-fucosyl- | 2′-FL, 3-FL, DFL, LNFP-I, |
| | | transferase | LNFP-III, LNFP-V, LNFP-VI, |
| | | | LNDFH-II, F-pLNH I |
| FucT_3_Am | ACD04596.1 | α-1,3-fucosyl- | 2′-FL, 3-FL, DFL, LNFP-I, |
| | | transferase | LNFP-III, LNFP-V, LNFP-VI, |
| | | | LNDFH-II, F-pLNH I |
| MAMA_R764 | AGC02224.1 | α-1,3-fucosyl- | 2′-FL, 3-FL, DFL, LNFP-I, |
| | | transferase | LNFP-III, LNFP-V, LNFP-VI, |
| | | | LNDFH-II, F-pLNH I |
| Mg791 | AEQ33441.1 | α-1,3-fucosyl- | 2′-FL, 3-FL, DFL, LNFP-I, |
| | | transferase | LNFP-III, LNFP-V, LNFP-VI, |
| | | | LNDFH-II, F-pLNH I |
| Moumou_00703 | YP_007354660 | α-1,3-fucosyl- | 2′-FL, 3-FL, DFL, LNFP-I, |
| | | transferase | LNFP-III, LNFP-V, LNFP-VI, |
| | | | LNDFH-II, F-pLNH I |
| futA | WP_000487428, | α-1,3-fucosyl- | 2′-FL, 3-FL, DFL, LNFP-I, |
| | SEQ ID NO: 26 | transferase | LNFP-III, LNFP-V, LNFP-VI, |
| | | | LNDFH-II, LNDFH-III, F-pLNH I |
| fucT | AAB81031.1, SEQ | α-1,3-fucosyl- | 2′-FL, 3-FL, DFL, LNFP-I, |
| | ID NO: 27 | transferase | LNFP-III, LNFP-V, LNFP-VI, |
| | | | LNDFH-II, LNDFH-III, F-pLNH I |
| fucTIII | AAR88243.1 | α-1,4-fucosyl- | LNDFH-I, LNDFH-II |
| | | transferase | |
| fucTa | AF194963.1 | α-1,3/4-fucosyl- | LNFP-II, LNDFH-I, LNDFH-II |
| | | transferase | |
| Pd2, 6ST | SEQ ID NO: 29 | α-2,6- | 6′-SL |
| | | sialyltransferase | |
| PspST6 | BAF92026.1 | α-2,6- | 6′-SL |
| | | sialyltransferase | |
| PiST6_145 | BAF91416.1 | α-2,6- | 6′-SL |
| | | sialyltransferase | |
| PiST6_119 | BAI49484.1 | α-2,6- | 6′-SL |
| | | sialyltransferase | |
| NST | SEQ ID NO: 30 | α-2,3- | 3′-SL |
| | | sialyltransferase | |
| Smob | WP_126455392.1, | α-1,2- | 2′-FL, LNFP-I |
| | SEQ ID NO: 24 | fucosyltransferase | |
| HD0466 | WP_010944479.1, | β-1,3-N- | LNT-II, LNT, LNnT, LNFP-I, |
| | SEQ ID NO: 22 | acetylglucosaminyl- | LNFP-II, LNFP-III, LNFP-V, |
| | | transferase | LNFP-VI, LNDFH-I, LNDFH-II, |
| | | | pLNH, F-pLNH I, pLNnH |
| PmnagT | WP_014390683.1, | β-1,3-N- | LNT-II, LNT, LNnT, LNFP-I, |
| | SEQ ID NO: 21 | acetylglucosaminyl- | LNFP-II, LNFP-III, LNFP-V, |
| | | transferase | LNFP-VI, LNDFH-I, LNDFH-II, |
| | | | pLNH, F-pLNH I, pLNnH |
| Cvb3galT | WP_080969100.1, | β-1,3- | LNT, LNFP-I, LNFP-II, LNFP-V, |
| | SEQ ID NO: 17 | galactosyltransferase | LNDFH-I, LNDFH-II, pLNH, F- |
| | | | PLNH I |
| Mtun | WP_031437198.1, | α-1,2- | 2′-FL, LNFP-I, LNDFH-I |
| | SEQ ID NO: 25 | fucosyltransferase | |

The above-mentioned enzymes enable the biosynthetic production of HMO core structures as well as fucosylated and/or sialylated HMO(s) and their glycosidic derivatives.

Production of neutral N-acetylglucosamine-containing HMOs in modified bacteria is known in the art (see e.g., Gebus C et al. (2012) Carbohydrate Research 363 83-90).

In one or more exemplary embodiment(s), the β-1,3-Gal-transferase(s) or β-1,4-gal-transferase(s) is/are selected from the group consisting of CvB3galT and GalTK, or GalT, respectively, and a functional homologue thereof, having an amino acid sequence which is at least 70% identical, such as at least 71, 72, 73, 74, 75, 76, 77, 78, 79, 80, 81, 82, 83, 84, 85, 86, 87, 88, 89, 90, 91, 92, 93, 94, 95, 96, 97, 98, 99 or 99.9% identical, to any one of SEQ ID NOs: 17, 18 or 19.

In one or more preferred exemplary embodiment(s), the β-1,3-Gal-transferase is CvB3galT with the amino acid sequence of SEQ ID NO: 17.

In one or more preferred exemplary embodiment(s) the β-1,3-Gal-transferase is GalTK with the amino acid sequence of SEQ ID NO: 18.

In another presently preferred embodiment, the β-1,4-gal-transferase is GalT with the amino acid sequence of SEQ ID NO: 19.

In one or more further exemplary embodiment(s), the β-1,3-GlcNAc-transferase(s) is/are selected from the group consisting of LgtA, PmnagT, HD0466 and a functional homologue thereof having an amino acid sequence which is at least 70% identical, such as at least 71, 72, 73, 74, 75, 76, 77, 78, 79, 80, 81, 82, 83, 84, 85, 86, 87, 88, 89, 90, 91, 92, 93, 94, 95, 96, 97, 98, 99 or 99.9% identical, to any one of SEQ ID NOs: 20, 21 or 22.

In one or more yet further exemplary embodiment(s), the β-1,3-GlcNAc-transferase is LgtA with the amino acid sequence of SEQ ID NO: 20.

In another one or more further exemplary disclosure(s), the α-1,2-fucosyl-transferase(s) is/are selected from the group consisting of FutC, Smob, Mtun and a functional homologue thereof having an amino acid sequence which is at least 70% identical, such as at least 71, 72, 73, 74, 75, 76, 77, 78, 79, 80, 81, 82, 83, 84, 85, 86, 87, 88, 89, 90, 91, 92, 93, 94, 95, 96, 97, 98, 99 or 99.9% identical, to any one of SEQ ID NOs: 23, 24 or 25.

In one or more further exemplary disclosure, the α-1,3-fucosyl-transferase(s) is/are selected from the group consisting of FutA, FucT, FucTIII and a functional homologue thereof having an amino acid sequence which is at least 70% identical, such as such as at least 71, 72, 73, 74, 75, 76, 77, 78, 79, 80, 81, 82, 83, 84, 85, 86, 87, 88, 89, 90, 91, 92, 93, 94, 95, 96, 97, 98, 99 or 99.9% identical, to any one of SEQ ID NO: 26, 27 or 28.

In a one or more yet further exemplary disclosure (s), the α-2,3-sialyl-transferase(s) is/are Pd2 and/or Nst and/or a functional homologue thereof having an amino acid sequence which is at least 70% identical, such as at least 71, 72, 73, 74, 75, 76, 77, 78, 79, 80, 81, 82, 83, 84, 85, 86, 87, 88, 89, 90, 91, 92, 93, 94, 95, 96, 97, 98, 99 or 99.9% identical to any one of SEQ ID NO: 29 or 30.

### Sucrose Fermentation

In the production of one or more HMOs, the starting material can have a great influence on the produced product, in terms of quality, purity and amount of product. The utilization of glucose as a carbon and energy source can in some cases be problematic due to difficulties in the handling and sterilization of glucose, which often leads to glucose degradation, formation of derivates and caramelization. Thus, the ability to utilize another sugar, such as but not limited to sucrose, with simpler handling parameters, is highly advantageous in the production of one or more HMOs.

In large scale manufacturing, the cost price and purity of starting materials is of vast importance for the viability of the production. Therefore, the utilization of the more resilient sugar sucrose, which comprises a glucose and a fructose moiety, is largely beneficial in large scale manufacturing. However, many manufacturing and/or production strains are incapable of utilizing sucrose as a carbon source due to lack of an enzyme capable of hydrolysing the sucrose. This has in some cases been overcome by addition of sucrose and a sucrose hydrolysing enzyme, such as an invertase, to the fermentation media, thus allowing the hydrolysis of sucrose into glucose and fructose.

In one or more preferred exemplary embodiment(s), the genetically engineered cell expresses a sucrose utilization system. Such a system can be endogenous to the cell, but it may also be heterologous if the cell is not capable of utilizing sucrose.

In one or more preferred exemplary embodiment(s), the genetically engineered cell comprises one or more heterologous nucleic acid sequence encoding one or more heterologous polypeptide(s), which enables utilization of sucrose as sole carbon and energy source of said genetically engineered cell.

In one or more preferred exemplary embodiment(s), the genetically engineered cell expresses a polypeptide capable of hydrolysing sucrose into glucose and fructose. Preferably, polypeptide capable of hydrolysing sucrose into glucose and fructose is a single heterologous enzyme.

Accordingly, in one or more exemplary embodiment(s), the heterologous polypeptide capable of hydrolysing sucrose into fructose and glucose expressed by the genetically modified cell of the present disclosure is selected from the group consisting of SEQ ID NO: 86 [SacC_Agal, glycoside hydrolase family 32 protein, WP_103853210.1] and SEQ ID NO: 87 [Bff, beta-fructofuranosidase protein, BAD18121.1], and a functional homologue of any one of SEQ ID NOs: 86 or 87, having an amino acid sequence which is at least 70% identical, such as at least 71, 72, 73, 74, 75, 76, 77, 78, 79, 80, 81, 82, 83, 84, 85, 86, 87, 88, 89, 90, 91, 92, 93, 94, 95, 96, 97, 98, 99 or 99.9% identical, to any one of SEQ ID NOs: 86 or 87.

Another approach is to express a heterologous sucrose utilization system, such as but not limited to expression of a PTS-dependent system or to express an invertase, enabling the cell to utilize sucrose as a carbon and energy source, which is described in examples 2 and 3 of the present disclosure.

A heterologous PTS-dependent sucrose utilization transport system containing a sucrose specific porin, a sucrose transport protein and a sucrose-6-phosphate hydrolase is e.g., described in WO2015197082. The oxidation of glucose-6-phosphate and fructose therein provides a biological energy source by the organism's own metabolic system. Also, glucose-6-phosphate and fructose serve as carbon source for producing sugar nucleotides in the cell's natural biosynthetic pathway. The so-produced sugar nucleotides are donors for glycosylating carbohydrate acceptors (e.g., lactose), internalized through a specific permease by the cell, and thereby manufacturing oligosaccharides of interest. The glycosylation is mediated by one or more glycosyl transferases which are directly produced by expressing heterologous genes. The organism lacks any enzyme degrading either the acceptor or the oligosaccharide product in the cell.

Thus, in one or more exemplary embodiment(s), the genetically engineered cell of the present disclosure comprises one or more heterologous nucleic acid sequence encoding one or more heterologous polypeptide(s) which enables utilization of sucrose as sole carbon and energy source of said genetically engineered cell. In one or more preferred exemplary embodiment(s), the genetically engineered cell comprises a PTS-dependent sucrose utilization system, further comprising the scrYA and scrBR operons.

Thus, in one or more exemplary embodiment(s), the genetically engineered cell according to the present disclosure comprises a PTS-dependent sucrose utilization transport system and/or a recombinant nucleic acid sequence encoding a heterologous polypeptide capable of hydrolysing sucrose into fructose and glucose.

In one or more exemplary embodiment(s), the polypeptide encoded by the scrYA operon are polypeptides with an amino acid sequence according to SEQ ID NOs: 88 and 89 [scrY and scrA] or a functional homologue of any one of SEQ ID NOs: 88 and 89 [scrY and scrA], having an amino acid sequence which is at least 80% identical, such as at least 90% identical such as at least 95% identical to any one of SEQ ID NO: 88 and 89 [scrY and scrA].

In one or more exemplary embodiment(s) the polypeptide encoded by the scrBR operon are polypeptides with an amino acid sequence according to SEQ ID NOs: 90 and 91 [scrB and scrR] or a functional homologue of any one of SEQ ID NOs: 90 and 91 [scrB and scrR], having an amino acid sequence which is at least 80% identical such as at least 90% identical such as at least 95% identical to any one of SEQ ID NOs: 90 and 91 [scrB and scrR].

Such cells as described above are capable of utilizing sucrose as carbon and energy source.

Further, the culturing step of the method(s) disclosed herein may comprise a two-step sucrose feeding, with a second feeding phase by continuously adding to the culture an amount of sucrose that is less than that added continuously in a first feeding phase, so as to slow the cell growth and increase the content of product produced in a high cell density culture.

The feeding rate of sucrose added continuously to the cell culture during the second feeding phase may be around 30-40% less than that of sucrose added continuously during the first feeding phase.

During both feeding phases, lactose can be added continuously, preferably with sucrose in the same feeding solution, or sequentially.

Optionally, the culturing further comprises a third feeding phase when considerable amount of unused acceptor remained after the second phase in the extracellular fraction.

Then the addition of sucrose is continued without adding the acceptor, preferably with around the same feeding rate set for the second feeding phase until consumption of the acceptor.

### Sugar Nucleotide Synthesis

The biosynthesis of activated sugar nucleotides can influence the production of one or more HMOs in several ways, firstly an enhanced production of specific sugar nucleotides that act as substrates in the HMO synthesis may enhance the overall HMO production or reduce the side product formation, secondly a reduction in specific sugar nucleotides may reduce the amount of side products produced while maintaining the level of produced target HMO. Thus, in one or more exemplary embodiment(s) of the present disclosure, the genetically engineered cell comprises at least one nucleic acid sequence encoding one or more heterologous polypeptides involved in the biosynthesis of activated sugars.

The term “activated sugars” or “activated sugar nucleotide” or “sugar nucleotide” are used interchangeably, and in the present context relates to modified sugars that act as precursors in the HMO synthesis, such as nucleotide sugars (e.g., UDP-galactose). In general monosaccharides are activated through conjugation to a nucleotide or phosphorylation, to be utilized in the glycosyltransferase mediated reactions, synthesizing the HMOs. Thus, modification of the sugar nucleotide synthesis is highly advantageous in the production of one or more HMOs.

An activated sugar nucleotide generally has a phosphorylated glycosyl residue attached to a nucleoside. A specific glycosyl transferase enzyme accepts only a specific activated sugar nucleotide. Thus, preferably the following activated sugar nucleotides are involved in the glycosyl transfer: glucose-UDP-GlcNAc, UDP-galactose, UDP-glucose, UDP-N-acetylglucosamine, UDP-N-acetylgalactosamine (GlcNAc) and CMP-N-acetylneuraminic acid. The genetically modified cell according to the present disclosure can comprise one or more pathways to produce a nucleotide-activated sugar selected from the group consisting of glucose-UDP-GlcNAc, GDP-fucose, UDP-galactose, UDP-glucose, UDP-N-acetylglucosamine, UDP-N-acetylgalactosamine and CMP-N-acetylneuraminic acid (CMP-Neu5Ac). In table 7 below are non-limiting examples of glycosyl-doners and the HMO products they can be used to produce, the list may not be exhaustive.

TABLE 7
glycosyl-donor HMO product list

| Glycosyl-donor | HMO product |
|---|---|
| UDP-GlcNAc | LNT, LNnT, LNFP-I, LNFP-II, LNFP-III, LNFP-V, |
| | LNFP-VI, LNDFH-I, LNDFH-II, LNDFH-III, LNH, |
| | LNnH, pLNH, pLNnH, F-pLNH-I, F-pLNH-II, |
| | F-pLNH-I, F-pLNnH-II, FLSTa, FLSTb, |
| | FLSTc, FLSTd, LSTa, LSTb, LSTc, LSTd, |
| | DSLNT, SLNH, SLNH-II |
| UDP-Gal | LNT, LNnT, LNFP-I, LNFP-II, LNFP-III, LNFP-V, |
| | LNFP-VI, LNDFH-I, LNDFH-II, LNDFH-III, LNH, |
| | LNnH, pLNH, pLNnH, F-pLNH-I, F-pLNH-II, |
| | F-pLNH-I, F-pLNnH-II, FLSTa, FLSTb, |
| | FLSTc, FLSTd, LSTa, |
| | LSTb, LSTc, LSTd, DSLNT, SLNH-I, SLNH-II |
| GDP-fucose | 2′FL, 3FL, DFL, LNFP-I, LNFP-II, LNFP-III, |
| | LNFP-V, LNFP-VI, LNDFH-I, LNDFH-II, LNDFH-III, |
| | F-LNH, F-LNnH, F-pLNH-I, F-pLNH-II, F-pLNH-I, |
| | F-pLNnH-II, FSL, FLSTa, FLSTb, FLSTc, FLSTd |
| CMP-Neu5Ac | 3′SL, 6′SL, FSL, FLSTa, FLSTb, FLSTc, |
| | FLSTd, LSTa, LSTb, LSTc, LSTd, DSLNT, |
| | SLNH-I, SLNH-II |

The present disclosure one or more exemplary embodiment(s) relates to a genetically modified cell which has a modified sugar nucleotide synthesis. In particular, an increased synthesis of the sugar nucleotide synthesis of UDP-GlcNAc and/or UDP-Gal may be advantageous in the production of LNT and/or LNnT.

In one or more exemplary disclosures(s), the genetically engineered cell expresses one or more polypeptides involved in the biosynthesis of activated sugar nucleotides. The one or more polypeptide involved in the biosynthesis of activated sugar nucleotides can be selected from the group consisting of Gmad, WcaG, WcaH, WcaJ, ManC, ManB, Pgm, GalU, GalE, GlmM, GlmU, GlmS, NeuA, NeuB, NeuC and a functional homologue thereof having an amino acid sequence which is at least 70% identical to any one of SEQ ID NOs: 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 and 45.

In one or more exemplary embodiment(s) of the present disclosure, the genetically engineered cell expresses one or more polypeptides involved in the biosynthesis of activated sugar nucleotides wherein the one or more polypeptide involved in the biosynthesis of activated sugar nucleotides is selected from the group consisting of Pgm [SEQ ID NO: 43 or 44], GalU [SEQ ID NO: 45 or 46], GalE [SEQ ID NO: 47 or 48], GlmM [SEQ ID NO: 49 or 50], GlmU [SEQ ID NO: 51 or 52], GlmS [SEQ ID NO: 33 or 54], and a functional homologue thereof having an amino acid sequence which is at least 70% identical to any one of SEQ ID NOs: 43, 44, 45, 46, 47, 48, 49, 50, 51, 52, 53, or 54.

### Colanic Acid Gene Cluster

The colanic acid gene cluster of Escherichia coli K-12 is responsible for the production of the extracellular polysaccharide colanic acid, a major oligosaccharide of the bacterial cell wall.

Since the colanic acid gene cluster is typically introduced in the genetically engineered cells as described in the examples, in the form of PglpF-driven expression cassettes, the deletion of the glpR gene (which codes the DNA-binding transcriptional repressor GlpR) eliminates the GlpR-imposed repression of transcription from all PglpF promoters in the cell and in this manner enhances gene expression from all PglpF-based cassettes, including the colanic acid gene cluster.

In one or more exemplary disclosure(s), the colanic acid gene cluster may be expressed from its native genomic locus. The expression may be actively modulated. The expression can be modulated by swapping the native promoter with a promoter of interest, and/or increasing the copy number of the colanic acid genes coding said protein(s) by expressing the gene cluster from another genomic locus than the native, or episomally expressing the colanic acid gene cluster or specific genes thereof.

Thus, in one or more exemplary disclosure (s), the expression of the colanic acid gene cluster is modulated by swapping the native promoter with a promoter of interest, and/or increasing the copy number of the colanic acid genes coding said protein(s) by expressing the gene cluster from another genomic locus than the native, or episomally expressing the colanic acid gene cluster.

In relation to the present disclosure, the term “native genomic locus”, in relation to the colanic acid gene cluster, relates to the original and natural position of the gene cluster in the genome of the genetically engineered cell.

### Gmd

The gmd gene encodes the protein GDP-mannose-4,6-dehydratase, which catalyzes the conversion of GDP-D-mannose to GDP-4-dehydro-6-deoxy-D-mannose. The protein is involved in the reaction that synthesizes GDP-L-fucose from GDP-alphα-D-mannose. In one or more exemplary disclosure (s), the gmd gene is over-expressed.

wcaG

The wcaG gene, also known as fel, encodes the protein GDP-L-fucose synthase (EC 1.1.1.271), which catalyses the two-step NADP-dependent conversion of GDP-4-dehydro-6-deoxy-D-mannose to GDP-fucose, involving an epimerase and a reductase reaction. In one or more exemplary disclosure (s), the wcaG gene is over-expressed.

wcaH

The wcaH gene encodes the protein GDP-mannose mannosyl hydrolase (EC 3.6.1.-), that hydrolyzes both GDP-mannose and GDP-glucose. In one or more exemplary disclosure (s), the wcaH gene is over-expressed.

wcaI

The wcaI gene encodes the colanic acid biosynthesis glycosyltransferase WcaI, and it catalyses the transfer of unmodified fucose to UPP-Glc (α-D-glucopyranosyl-diphosphoundecaprenol-glucose). In one or more exemplary disclosure (s), the wcaI gene is over-expressed.

manB

The manB gene encodes the protein phosphomannomutase (EC 5.4.2.8), which is involved in the biosynthesis of GDP-mannose by catalysing conversion α-D-mannose-1-phosphate into D-mannose-6-phosphate. Thus, the expression level of manB regulates the formation of GDP-mannose. In one or more exemplary disclosure (s), the manB gene is over-expressed.

manC

The manC gene encodes the protein mannose-l-phosphate guanylyltransferase (EC 2.7.7.13), that is involved in the biosynthesis of GDP-mannose through synthesis of GDP-mannose from GTP and α-D-mannose-1-phosphate. In one or more exemplary disclosure (s), the manC gene is over-expressed.

### Sialic Acid Catabolic Pathway

Furthermore, the genetically engineered cell may also comprise a sialic acid synthetic capability. For example, the cell comprises a sialic acid synthetic capability through provision of an exogenous UDP-GlcNAc 2-epimerase (e.g., neuC of Campylobacter jejuni (GenBank AAK91727.1) or equivalent (e.g., (GenBank CAR04561.1), a Neu5Ac synthase (e.g., neuB of C. jejuni (GenBank AAK91726.1) or equivalent, (e.g., Flavobacterium limnosediminis sialic acid synthase, NCBI reference sequence WP_023580510.1), and/or a CMP-Neu5Ac synthetase (e.g., neuA of C. jejuni (GenBank AAK91728.1) or equivalent, (e.g., Vibrio brasiliensis CMP-sialic acid synthase, NCBI reference sequence WP_006881452.1).

In one or more exemplary disclosure (s), the genetically engineered cell, contains a deficient sialic acid catabolic pathway. By “sialic acid catabolic pathway” is meant a sequence of reactions, usually controlled and catalyzed by enzymes, which results in the degradation of sialic acid. An exemplary sialic acid catabolic pathway described herein is the E. coli pathway. In this pathway, sialic acid (Neu5Ac; N-acetylneuraminic acid) is degraded by the enzymes NanA (N-acetylneuraminic acid lyase) and NanK (N-acetylmannosamine kinase) and NanE (N-acetylmannosamine-6-phosphate epimerase), all encoded from the nanATEK-yhcH operon, and repressed by NanR (http://ecocyc.org/ECOLI). A deficient sialic acid catabolic pathway is rendered in the E. coli host by introducing a mutation in the endogenous nanA (N-acetylneuraminate lyase) (e.g., GenBank Accession Number D00067.1(GL216588)) and/or nanK (N-acetylmannosamine kinase) genes (e.g., GenBank reference (amino acid) BAE77265.1 (GL85676015)), and/or nanE (N-acetylmannosamine-6-phosphate epimerase, GeneID: 947745, incorporated herein by reference). Optionally, the nanT (N-acetylneuraminate transporter) gene is also inactivated or mutated. Other intermediates of sialic acid metabolism include: (ManNAc-6-P) N-acetylmannosamine-6-phosphate; (GlcNAc-6-P) N-acetylglucosamine-6-phosphate; (GlcN-6-P) Glucosamine-6-phosphate, and (Fruc-6-P) Fructose-6-phosphate.

In one or more preferred exemplary disclosure (s), nanA is mutated. In other preferred embodiment(s), nanA and nanK are mutated, while nanE remains functional. In another preferred embodiment, nanA and nanE are mutated, while nanK has not been mutated, inactivated or deleted. A mutation in the present context refers to one or more changes in the nucleic acid sequence coding the gene product of nanA, nanK, nanE, and/or nanT. For example, the mutation may be 1, 2, up to 5, up to 10, up to 25, up to 50 or up to 100 changes in the nucleic acid sequence. For example, the nanA, nanK, nanE, and/or nanT genes are mutated by a null mutation. Null mutations as described herein encompass amino acid substitutions, additions, deletions, or insertions, which either cause a loss of function of the enzyme (i.e., reduced or no activity) or loss of the enzyme (i.e., no gene product). By “deleted” is meant that the coding region is removed completely or in part such that no (functional) gene product is produced. By inactivated is meant that the coding sequence has been altered such that the resulting gene product is functionally inactive or encodes for a gene product with less than 100%, e.g., 90%, 80%, 70%, 60%, 50%, 40%, 30% or 20% of the activity of the native, naturally occurring, endogenous gene product. A “not mutated” gene or protein does not differ from a native, naturally occurring, or endogenous coding sequence by 1, 2, up to 5, up to 10, up to 20, up to 50, up to 100, up to 200 or up to 500 or more codons, or to the corresponding encoded amino acid sequence.

### Repressors

In one or more exemplary embodiment(s), the genetically engineered cell disclosed herein comprises a non-functional or absent gene product that normally binds to a regulatory element and represses the expression of any of the proteins of the present disclosure regulated by said regulatory element.

The term a non-functional (or absent) gene product that normally binds to and represses the expression driven by the regulatory element in the present context relates to DNA binding sites upstream of the coding sequence of a gene of interest and specifically at the promoter region of said gene.

In one or more exemplary embodiments, the cell may have a non-functional (or absent) gene product(s) that would normally bind to and repress the expression of the lacY gene and/or any of the proteins having an amino acid sequence of SEQ ID NOs: 1, 2, 3, 4, 5, 6, 7, 8, 9, 10, 11, 12, 13, 14, 15 or 16, or regions upstream of the regulatory element for controlling the expression of the lacY gene and/or any of the proteins having an amino acid sequence of SEQ ID NOs: 1, 2, 3, 4, 5, 6, 7, 8, 9, 10, 11, 12, 13, 14, 15 or 16.

In one or more exemplary embodiments, said gene product is the DNA-binding transcriptional repressor GlpR.

### GlpR

GlpR belongs to the DeoR family of transcriptional regulators and acts as the repressor of the glycerol-3-phosphate regulon, which is organized in different operons. This regulator is part of the glpEGR operon, yet it can also be constitutively expressed as an independent (glpR) transcription unit. In addition, the operons regulated are induced when Escherichia coli is grown in the presence of inducer, glycerol, or glycerol-3-phosphate (G3P), and the absence of glucose. In the absence of inducer, this repressor binds in tandem to inverted repeat sequences that consist of 20-nucleic acid-long DNA target sites.

The term “non-functional or absent” in relation to the glpR gene refers to the inactivation of the glpR gene by complete or partial deletion of the corresponding nucleic acid sequence from the bacterial genome (e.g. SEQ ID NO: 92 or variants thereof encoding glpR capable of downregulating glpF derived promoters). The glpR gene encodes the DNA-binding transcriptional repressor GlpR. In this way promoter sequences of the PglpF family are upregulated in the genetically engineered cell, due to deletion of the repressor gene that would otherwise downregulate the PglpF promoters.

In one or more exemplary embodiment(s), the glpR gene is deleted.

### Activators

In one or more exemplary embodiment(s), the genetically engineered cell disclosed herein comprises an over-expressed gene product that enhances the expression of any of the genes and/or proteins of the present disclosure regulated by said regulatory element.

In one or more exemplary embodiments, the cell of the present disclosure may comprise an over-expressed gene product that enhances the expression of the gene(s) encoding the lactose permease LacY and/or any of the proteins having an amino acid sequence of SEQ ID NOs: 1-16.

In one or more exemplary embodiments, said gene product is the cAMP DNA-binding transcriptional dual regulator CRP.

### CRP

CRP belongs to the CRP-FNR superfamily of transcription factors. CRP regulates the expression of several of the E. coli genes, many of which are involved in catabolism of secondary carbon sources. Upon activation by cyclic-AMP, (cAMP) CRP binds directly to specific promoter sequences, the binding recruits the RNA polymerase through direct interaction, which in turn activates the transcription of the nucleic acid sequence following the promoter sequence leading to expression of the gene of interest. Thus, over-expression of CRP may lead to an enhanced expression of a gene/nucleic acid sequence of interest. Amongst other functions, CRP exerts its function on the PglpF promoters, where it contrary to the repressor GlpR, activates promoter sequences of the PglpF family. In this way, over-expression of CRP in the genetically engineered cell of the present disclosure, promotes expression of genes that are regulated by promoters of the PglpF family.

Thus, in one or more exemplary embodiments, the crp gene is over-expressed.

Genetic engineering of GlpR and/or CRP, as suggested in the present disclosure, in 2′-FL producing strains is beneficial for the overall production of 2′-FL by these strains.

### Regulator Element

The genetically engineered cell according to present disclosure may comprise regulatory elements enabling the controlled overexpression of endogenous, heterologous and/or synthetic nucleic acid sequences.

The term “regulatory element”, comprises promoter sequences, signal sequence, and/or arrays of transcription factor binding sites, which sequences affect transcription and/or translation of a nucleic acid sequence operably linked to the regulatory element.

Regulatory elements are found at transcriptional and post-transcriptional levels and further enable molecular networks at those levels. For example, at the post-transcriptional level, the biochemical signals controlling mRNA stability, translation and subcellular localization are processed by regulatory elements. RNA binding proteins are another class of post-transcriptional regulatory elements and are further classified as sequence elements or structural elements. Specific sequence motifs that may serve as regulatory elements are also associated with mRNA modifications. A variety of DNA regulatory elements are involved in the regulation of gene expression and rely on the biochemical interactions involving DNA, the cellular proteins that make up chromatin, and transcription factors.

In general, the transcriptional and translational regulatory sequences include, but are not limited to, promoter sequences, ribosomal binding sites, transcriptional start and stop sequences, translational start and stop sequences, and enhancer or activator sequences.

Promoters and enhancers are the primary genomic regulatory components of gene expression. Promoters are DNA regions within 1-2 kilobases (kb) of a gene's transcription start site (TSS); they contain short regulatory elements (DNA motifs) necessary to assemble RNA polymerase transcriptional machinery. However, transcription is often minimal without the contribution of DNA regulatory elements located more distal to the TSS. Such regions, often termed enhancers, are position-independent DNA regulatory elements that interact with site-specific transcription factors to establish cell type identity and regulate gene expression. Enhancers may act independently of their sequence context and at distances of several to many hundreds of kb from their target genes through a process known as looping. As a consequence of these features, it is difficult to identify suitable enhancers and link them to their target genes on the basis of DNA sequence alone.

The promoter, together with other transcriptional and translational regulatory nucleic acid sequences (also termed “control sequences”) is necessary to express a given gene or group of genes (an operon).

Identification of suitable promoter sequences that promotes expression of the specific gene of interest is a tedious task, which in many cases require laborious efforts. In relation to the present disclosure regulator elements may or may not be post-translational regulators or it may or may not be translational regulators.

In one or more exemplary embodiment(s) of the invention, the expression of any one or more nucleic acid sequence(s) and/or genes is/are regulated by one or more regulatory element(s).

In one or more exemplary embodiment(s), the regulatory element comprises one or more elements capable of enhancing the expression, i.e., overexpression of any one or more nucleic acid sequence(s) according to the present disclosure.

In that regard, the regulatory element may be endogenous or heterologous, and/or recombinant and/or synthetic nucleic acid sequences. In the present context, the term “heterologous regulatory element” is to be understood as a regulatory element that is not endogenous to the original, genetically engineered cell described herein. The heterologous regulatory element may also be a recombinant regulatory element, wherein two or more non-operably linked native regulatory element(s) are recombined into a heterologous and/or synthetic regulatory element. The heterologous regulatory element, may be introduced into the genetically engineered cell using methods known to the person skilled in the art.

The regulatory element may be a native regulatory element, such as e.g., the Plac promoter, promoting the expression of the lac operon native to E. coli.

### Promoter Sequences

The present disclosure relates to a genetically engineered cell and/or a method comprising providing said genetically engineered cell, wherein the expression of any one or more of said nucleic acid sequence(s) is/are regulated by one or more regulatory element(s). Said regulatory element can comprise one or more promoter sequence(s).

The regulatory element or elements regulating the expression of the genes and/or nucleic acid sequence(s), may comprise one or more promoter sequence(s), wherein the promoter sequence, is operably linked to the nucleic acid sequence of the gene of interest in that sense regulating the expression of the nucleic acid sequence of the gene of interest.

In general, a promoter may comprise native, heterologous and/or synthetic nucleic acid sequences, and may be a recombinant nucleic acid sequence, recombining two or more nucleic acid sequences or same or different origin as described above, thereby generating a homologous, heterologous, or synthetic nucleic promoter sequence, and/or a homologous, heterologous, or synthetic nucleic regulatory element. A wide selection of promoter sequences derived from the PglpF, PlgpA, PlgpT, PgatY, PmglB and Plac promoter systems are described in detail WO2019123324A1 and WO2020255054A1.

In one or more exemplary embodiment(s), the regulatory element of the genes and/or heterologous nucleic acid sequences of the genetically engineered cell comprises more than one native or heterologous promoter sequence.

In one or more exemplary embodiment(s), the regulatory element of the genes and/or heterologous nucleic acid sequences of the genetically engineered cell comprises two or more regulatory elements with identical promoter sequences.

In one or more exemplary embodiment(s), the regulatory element of the genes and/or heterologous nucleic acid sequences of the genetically engineered cell comprises two or more regulatory elements with non-identical promoter sequences.

The regulatory architectures i.e., gene-by-gene distributions of transcription-factor-binding sites and identities of the transcription factors that bind those sites can be used in multiple different growth conditions and there are more than 100 genes from across the E. coli genome, which act as regulatory elements. Thus, any promoter sequence enabling transcription and/or regulation of the level of transcription, of one or more heterologous or native nucleic acid sequences that encode one or more proteins as described herein may be suitable.

One way to increase the production of a product may be to regulate the production of the desired enzyme activity used to produce the product or precursor/substrate import, such as the glycosyltransferases or the MFS transporter or the lactose permease.

Increasing the promoter strength driving the expression of the desired enzyme may be one way of doing this. The strength of a promoter can be assed using a lacZ enzyme assay where β-galactosidase activity is assayed as described previously (see e.g. Miller J. H. Experiments in molecular genetics, Cold spring Harbor Laboratory Press, NY, 1972). Briefly the cells are diluted in Z-buffer and permeabilized with sodium dodecyl sulfate (0.1%) and chloroform. The LacZ assay is performed at 30° C. Samples are preheated, the assay initiated by addition of 200 μl ortho-nitro-phenyl-β-galactosidase (4 mg/ml) and stopped by addition of 500 μl of 1 M Na₂CO₃ when the sample had turned slightly yellow. The release of ortho-nitrophenol is subsequently determined as the change in optical density at 420 nm. The specific activities are reported in Miller Units (MU) [A420/(min*ml*A600)]. A regulatory element with an activity above 10,000 MU is considered strong and a regulatory element with an activity below 3,000 MU is considered weak, what is in between has intermediate strength. An example of a strong regulatory element is the PglpF promoter with an activity of approximately 14.000 MU and an example of a weak promoter is Plac which when induced with IPTG has an activity of approximately 2300 MU.

Alternatively, if there is a need for balancing the expression level of one or more proteins to optimize the production it may be beneficial to use a promoter with the desired strength, e.g., middle or low strength. Table 8 below lists a series of wildtype and recombinant promoters according to their strength relative to the PglpF promoter.

TABLE 8
Promoter sequences according to strength

| | % activity | | |
|---|---|---|---|
| | relative to | | Seq ID |
| Promoter name | PglpF* | Strength | in appl. |
| PmglB_70UTR_SD8 | 291% | high | 93 |
| PmglB_70UTR_SD10 | 233-281% | high | 94 |
| PmglB_54UTR | 197% | high | 95 |
| Plac_70UTR | 182-220% | high | 96 |
| PmglB_70UTR_SD9 | 180-226% | high | 97 |
| PmglB_70UTR_SD4 | 153%-353% | high | 83 |
| PmglB_70UTR_SD5 | 146-152% | high | 98 |
| PglpF_SD4 | 140-161% | high | 75 |
| PmglB_70UTR_SD7 | 127-173% | high | 99 |
| PmglB_70UTR | 124-234% | high | 82 |
| PglpA_70UTR | 102-179% | high | 100 |
| PglpT_70UTR | 102-240% | high | 101 |
| pgatY_70UTR | 112% | high | 69 |
| PglpF | 100% | high | 70 |
| PglpF_SD10 | 88-96% | high | 72 |
| PglpF_SD5 | 82-91% | high | 76 |
| PglpF_SD8 | 81-82% | high | 79 |
| PmglB_16UTR | 78-171% | high | 102 |
| PglpF_SD9 | 73-93% | middle | 80 |
| PglpF_SD7 | 47-57% | middle | 78 |
| PglpF_SD6 | 46-47% | middle | 77 |
| PglpA_16UTR | 38-64% | middle | 103 |
| Plac | 15-28% | low | 68 |
| PglpF_SD3 | 9% | low | 74 |
| PglpF_SD1 | 5% | low | 71 |
| PglpF_SD2 | na | | 73 |
| Plac_16UTR | | | 81 |
| Cp6 | na | | 84 |
| PosmY | na | | 85 |

na = not accessed
*The promoter activity is assessed in the LacZ assay described below with the PglpF promoter run as positive reference in the same assay. To compare across assays the activity is calculated relative to the PglpF promoter, a range indicates results from multiple assays

The promoter may be of heterologous origin, native to the genetically modified cell or it may be a recombinant promoter, combining heterologous and/or native elements.

In one or more exemplary embodiment(s), the regulatory element comprises a promoter sequence selected from the group consisting of PBAD, Pxyl, Plac, PsacB, PxylA, PrpR, PnitA, PT7, Ptac, PL, PR, PnisA, Pb, PgatY_70UTR, PglpF, PglpF_SD1, PglpF_SD10, PglpF_SD2, PglpF_SD3, PglpF_SD4, PglpF_SD5, PglpF_SD6, PglpF_SD7, PglpF_SD8, PglpF_SD9, Plac_16UTR, PmglB_70UTR, PmglB_70UTR_SD4, CP6, PosmY, Pspc, Pbla, Prrn1 and Prrn2.

In a currently preferred embodiment, the promoter sequence is selected from the group consisting of PgatY_70UTR, PglpF, PglpF_SD1, PglpF_SD10, PglpF_SD2, PglpF_SD3, PglpF_SD4, PglpF_SD5, PglpF_SD6, PglpF_SD7, PglpF_SD8, PglpF_SD9, Plac_16UTR, and Plac.

In one or more exemplary embodiments, the regulatory element is a promoter selected from the group consisting of PglpF (SEQ ID NO: 70), PglpT 70UTR (SEQ ID NO: 101), SEQ ID NO: 69 (PgatY_70UTR), Plac (SEQ ID NO: 68, PmglB_70UTR (SEQ ID NO: 82), PglpA_70UTR (SEQ ID NO: 100), Cp6 (SEQ ID NO: 84), Posmy (SEQ ID NO: 85) or variants of these, and variants thereof. Specifically, the variants disclosed in table 8 are preferred.

In one or more exemplary embodiments, the regulatory element is a promoter with high or middle strength, such as a promoter sequence selected from the group consisting of PmglB_70UTR_SD8, PmglB_70UTR_SD10, PmglB_54UTR, Plac_70UTR, PmglB_70UTR_SD9, PmglB_70UTR_SD4, PmglB_70UTR_SD5, PglpF_SD4, PmglB_70UTR_SD7, PmglB_70UTR, PglpA_70UTR, PglpT_70UTR, pgatY_70UTR, PglpF, PglpF_SD10, PglpF_SD5, PglpF_SD8, PglpF_B28, PglpF_B29, PmglB_16UTR, PglpF_SD9, PglpF_SD7, PglpF_SD6 and PglpA_16UTR.

In one preferred embodiment the promoter is a strong promoter selected from the group consisting of PmglB_70UTR_SD8, PmglB_70UTR_SD10, PmglB_54UTR, Plac_70UTR, PmglB_70UTR_SD9, PmglB_70UTR_SD4, PmglB_70UTR_SD5, PglpF_SD4, PmglB_70UTR_SD7, PmglB_70UTR, PglpA_70UTR, PglpT_70UTR, pgatY_70UTR, PglpF, PglpF_SD10, PglpF_SD5, PglpF_SD8, and PmglB_16UTR. This may in particular be advantageous for the expression the heterologous glycosyltransferase and or MFS transporter.

In another embodiment the promoter is selected from the group consisting of promoters with middle strength, such as PglpF_SD9, PglpF_SD7, PglpF_SD6 and PglpA_16UTR, This may in particular be advantageous for the expression the heterologous MFS transporter, if there is a need to balance the expression towards the expression of the lactose permease.

In another embodiment the promoter is selected from the group consisting of promoters with low strength, such as Plac, PglpF_SD3 and PglpF_SD1. This may in particular be advantageous for the expression the heterologous MFS transporter, if there is a need to balance the expression towards the expression of the lactose permease.

In a currently preferred embodiment, the promoter sequence is PglpF as shown in SEQ ID NO: 70. In a preferred embodiment of the invention, a promoter element of middle or low strength, such as the Plac promoter (SEQ ID NO: 68) is comprised in the regulatory element regulating the heterologous MFS transporter and a promoter of high strength, such as the PglpF (SEQ ID NO: 70) is comprised in the regulatory element regulating LacY.

In a currently preferred embodiment, the Plac promoter element of SEQ ID NO: 68 is comprised in the regulatory element regulating YberC and PglpF as shown in SEQ ID NO: 70 is comprised in the regulatory element regulating LacY

An example of another type of regulatory elements is a Shine-Dalgarno sequence, which is a ribosomal binding site in bacterial and archaeal messenger RNA, generally located around 8 bases upstream of the start codon AUG, which helps recruiting the ribosome to the mRNA and initiate protein synthesis. Thus, modification of the Shine-Dalgarno sequence upstream of the coding nucleic acid sequence may modify the expression level of the any one or more of the genes and/or nucleic acid sequences and/or polypeptides of the invention.

Accordingly, in one or more exemplary embodiment(s), the genetically engineered cell according to the present disclosure comprises one or more endogenous, heterologous, synthetic and/or optimized Shine-Dalgarno sequence(s).

### Sequence Identity

The term “sequence identity of [a certain]%” in the context of two or more nucleic acid or amino acid sequences means that the two or more sequences have nucleic acids or amino acid residues in common in the given percent, when compared and aligned for maximum correspondence over a comparison window or designated sequences of nucleic acids or amino acids (i.e., the sequences have at least 90 percent (%) identity). Percent identity of nucleic acid or amino acid sequences can be measured using a BLAST 2.0 sequence comparison algorithm with default parameters, or by manual alignment and visual inspection (see e.g., http://www.ncbi.nlm.nih.gov/BLAST/). This definition also applies to the complement of a test sequence and to sequences that have deletions and/or additions, as well as those that have substitutions. An example of an algorithm that is suitable for determining percent identity, sequence similarity and for alignment is the BLAST 2.2.20+ algorithm, which is described in Altschul et al. Nucl. Acids Res. 25, 3389 (1997). BLAST 2.2.20+ is used to determine percent sequence identity for the nucleic acids and proteins of the disclosure. Software for performing BLAST analyses is publicly available through the National Center for Biotechnology Information (http://www.ncbi.nlm.nih.gov/). Examples of commonly used sequence alignment algorithms are

CLUSTAL Omega (http://www.ebi.ac.uk/Tools/msa/clustalo/),
EMBOSS Needle (http://www.ebi.ac.uk/Tools/psa/emboss_needle/),
MAFFT (http://mafft.cbrc.jp/alignment/server/), or
MUSCLE (http://www.ebi.ac.uk/Tools/msa/muscle/).

For purposes of the present invention, the sequence identity between two amino acid sequences is preferably determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277,), preferably version 5.0.0 or later (available at https://www.ebi.ac.uk/Tools/psa/emboss needle/). The parameters used are gap open penalty of 10, gap extension penalty of 0.5, and the EBLOSUM62 (EMBOSS version of 30 BLOSUM62) substitution matrix. The output of Needle labeled “longest identity” (obtained using the—nobrief option) is used as the percent identity and is calculated as follows: (Identical Residues×100)/(Length of Alignment−Total Number of Gaps in Alignment).

For purposes of the present invention, the sequence identity between two nucleotide sequences is preferably determined using the Needleman-Wunsch algorithm (Needleman and Wunsch, 1 970, supra) as implemented in the Needle program of the EMBOSS package (EMBOSS: The European Molecular Biology Open Software Suite, Rice et al., 2000, Trends Genet. 16: 276-277), 10 preferably version 5.0.0 or later. The parameters used are gap open penalty of 10, gap extension penalty of 0.5, and the DNAFULL (EMBOSS version of NCBI NUC4.4) substitution matrix. The output of Needle labeled “longest identity” (obtained using the—nobrief option) is used as the percent identity and is calculated as follows: (Identical Deoxyribonucleotides×100)/(Length of Alignment Total Number of Gaps in Alignment).

### Functional Homologues

A functional homologue of a protein/nucleotide as described herein is a protein/nucleotide with alterations in the genetic code, which retains its original functionality. A functional homologue may be obtained by mutagenesis. The functional homologue should have a remaining functionality of at least 50%, such as 60%, 70%, 80%, 90% or 100% compared to the functionality of the protein/nucleotide.

A functional homologue of any one of the disclosed amino acid sequences can also have a higher functionality. A functional homologue of any one of the herein disclosed polypeptides, should ideally be able to participate in the HMO production, in terms of HMO yield, purity, reduction in biomass formation, viability of the genetically engineered cell, robustness of the genetically engineered cell according to the disclosure, or reduction in consumables.

Integrated into the Genome

The nucleic acid sequence(s) expressed in the genetically engineered cell according to the present disclosure may be integrated into the genome of the genetically engineered cell.

Integration of the heterologous nucleic acid of interest, potentially comprised in a construct (expression cassette), into the genome can be achieved by conventional methods, e.g., by using linear cartridges that contain flanking sequences homologous to a specific site on the chromosome, as described for the attTn7-site (Waddell C. S. and Craig N. L., Genes Dev. (1988) Feb;2(2):137-49.); methods for genomic integration of nucleic acid sequences in which recombination is mediated by the Red recombinase function of the phage, or the RecE/RecT recombinase function of the Rac prophage (Murphy, J Bacteriol. (1998);180(8):2063-7; Zhang et al., Nature Genetics (1998) 20: 123-128; Muyrers et al., EMBO Rep. (2000) 1(3): 239-243); methods based on Red/ET recombination (Wenzel et al., Chem Biol. (2005), 12(3):349-56; Vetcher et al., Appl Environ Microbiol. (2005);71(4):1829-35); or positive clones, i.e., clones that carry the expression cassette, can be selected e.g., by means of a marker gene, or loss or gain of gene function.

Expression and/or overexpression of the native or heterologous genes may one or more exemplary embodiment(s) be obtained from episomal integration of said genes, i.e., the nucleic acid encoding the heterologous polypeptide may be inserted into a nucleic acid construct, which is then subsequently comprised in a plasmid or in another chromosomal independent expression vector, wherein the expression vector at some instances is capable of integrating into the chromosome, however not be required to do so in order to obtain expression of the heterologous nucleic acid encoding a heterologous polypeptide of the invention. Examples of episomes are for instance transposons and insertion sequences.

In the scope of the invention as disclosed, the term “genomically integrated” refers to the integration of one or more native or heterologous nucleic acid sequences into the chromosome or into an endogenous plasmid, thus being integrated into the genome of the genetically engineered cell of the invention.

Thus, in one or more exemplary preferred embodiment, the invention relates to a genetically engineered cell as described above, which comprises one or more heterologous nucleic acid sequence(s) encoding one or more episomal and/or genomically integrated copies of e.g., one or more lactose permease(s) and/or a heterologous MFS transporter gene and/or one or more glycosyltransferases as described above.

In one or more exemplary embodiment(s), the one or more native and/or heterologous nucleic acid sequence(s) encoding e.g., one or more lactose permease(s) and/or a heterologous MFS transporter and/or one or more glycosyltransferases according to the invention, is/are integrated into the genome of the genetically engineered cell in a stable manner.

In one or more further exemplary embodiment(s), the one or more native and/or heterologous nucleic acid sequence(s) encoding e.g., one or more lactose permease(s) and/or a heterologous MFS transporter and/or one or more glycosyltransferases according to the invention, is/are integrated into the genome of the genetically engineered cell in a transiently manner.

Expressed from a Plasmid

In one or more further exemplary embodiment(s), the one or more nucleic acid sequence(s) is/are expressed from a plasmid.

In the present disclosure, one or more nucleic acid sequences encoding e.g., one or more lactose permease(s) and/or a heterologous MFS transporter and/or one or more glycosyltransferases, according to the invention, can be introduced to the genetically engineered cell in a transient manner.

Thus, in one or more exemplary embodiment(s) one or more heterologous nucleic acid sequence(s) which e.g., encodes one or one or more lactose permease(s) and/or a heterologous MFS transporter gene and/or one or more glycosyltransferases is/are plasmid-borne, such as but not limited to a low, medium, or high copy number plasmid.

In the present disclosure, low/medium/high copy number plasmid relates to the number of plasmid copies per bacterial cell. In one or more exemplary embodiment(s) of the present disclosure, a low copy number plasmid is a plasmid wherein the cell comprises on average 1-20 copies of said plasmid, such as on average 1, 2, 3, 4, 5, 6, 7, 8, 9, 10, 11, 12, 13, 14, 15, 16, 17, 18, 19 or 20 copies per cell. Accordingly, in one or more exemplary embodiment(s) of the present disclosure, a medium copy number plasmid is a plasmid wherein the cell comprises on average 21-100 copies of said plasmid, such as on average 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 copies per cell. Further, in one or more exemplary embodiment(s) of the present disclosure, a high copy number plasmid is a plasmid wherein the cell comprises at least on average 101 copies of said plasmid, such as on average at least 200, 300, 400, 500, 600, 700, 800, 900 or 1000 copies per cell.

### Cell Culture Medium

In the present context, a growth medium or culture medium is a liquid or gel designed to support the growth of microorganisms, cells, or small plants. The medium comprises an appropriate source of energy and may comprise compounds which regulate the cell cycle. The culture medium may be semi-defined, i.e., containing complex media compounds (e.g., yeast extract, soy peptone, casamino acids, etc.), or it may be chemically defined, without any complex compounds. Exemplary suitable media are provided in the experimental examples.

In one or more exemplary embodiment(s), the culturing media is minimal media.

In one or more exemplary embodiment(s), the culturing media is supplemented with one or more energy and carbon sources selected form the group containing glycerol, sucrose, glucose, and fructose.

In one or more exemplary embodiment(s), the culturing media is supplemented with one or more energy and carbon sources selected form the group containing glycerol, sucrose, and glucose.

In one or more exemplary embodiment(s), the culturing media is supplemented with glycerol, sucrose and/or glucose.

In one or more exemplary embodiment(s), the culturing media is supplemented with glycerol and/or glucose.

In one or more exemplary embodiment(s), the culturing media is supplemented with sucrose and/or glucose.

In one or more exemplary embodiment(s), the culturing media is supplemented with glycerol and/or sucrose.

In one or more exemplary embodiment(s), the culturing media is supplemented only with sucrose.

In one or more exemplary embodiment(s), the culturing media contains sucrose as the sole carbon and energy source.

In one or more exemplary embodiment(s), the culturing media is supplemented with lactose as a suitable acceptor for the glycosyl transferases of the invention.

### Culturing of Said Cell

The present disclosure relates to a method for producing HMO in a bio-synthetic process, comprising culturing a genetically engineered cell according to the present disclosure in a suitable media and harvesting the one or more HMO(s). Preferably the medium contains lactose.

According to the invention, the term “culturing” (or “cultivating” or “cultivation”, also termed “fermentation”) relates to the propagation of bacterial expression cells in a controlled bioreactor according to methods known in the industry.

According to said method the genetically engineered cell, preferably a bacterium, more preferably an E. coli, is a strain that is optimized for an industrially profitable transformation like HMO production. Such an optimization may comprise the steps described in the present disclosure.

To produce one or more HMO(s), the HMO-producing microorganism as described herein are cultivated according to the procedures known in the art and the produced HMO is harvested from the cultivation media and the biomass formed during the cultivation process. Thereafter, the HMO(s) are purified according to the procedures known in the art, e.g., such as described in WO2015188834, WO2017182965 or WO2017152918, and the purified HMO(s) are used as nutraceuticals, pharmaceuticals, or for any other purpose, e.g., for research.

During culturing of the genetically engineered cell, disclosed herein, the oligosaccharide-producing cell is fed with a carbon and energy source, such as but not limited to sucrose, that provides energy via glycolysis for growing, reproducing, and maintaining its structure. In addition, the energy and carbon source taken up by the cell provides or acts as precursors for the synthesis of the activated sugar nucleotide(s) necessary for the glycosylation process that takes place in the cell, as described above. Additionally, a lactose solution is added to the media.

As is demonstrated in example 1, lactose can be provided as a bolus injection of 20% lactose solution (0.1 ml) to a basal minimal medium supplemented with magnesium sulphate and thiamine. Alternatively, or in addition, lactose can be added continuously, preferably with sucrose in the same feeding solution, or sequentially.

### Level of Lactose During Culturing

In one or more exemplary embodiment(s), the level of lactose in the culturing media is modulated.

In one or more exemplary embodiment(s), the level of lactose during the culturing of the genetically engineered cell is modulated from low to high. In the present context this results in the following lactose concentration ranges: high lactose process 30-80 g/L, low lactose process 0-15 g/L, such as 0.5-15 g/L.

In one or more exemplary embodiment(s), a high level of lactose level relates to 30-80 g/L, such as but not limited to 30-40 g/L, 30-50 g/L, 30-60 g/L, 30-70 g/L, 40-50 g/L, 40-60 g/L, 40-70 g/L, 40-80 g/L, 50-60 g/L, 50-70 g/L, 50-80 g/L, 60-70 g/L, 60-80 g/L, 35-50 g/L, 35-60 g/L, 35-70 g/L, 35-75 g/L, 35-80 g/L, 45-55 g/L, 45-75 g/L, 55-65 g/L, 55-75 g/L, 55-80 g/L, 65-75 g/L, or 65-80 g/L.

In one or more exemplary embodiment(s), a low level of lactose level relates to 0-15 g/L, such as but not limited to 0-5 g/L, 0-7.5 g/L, 0-10 g/L, 0-12.5 g/L, 0.5-5 g/L, 0.5-7.5 g/L, 0.5-10 g/L, 0.5-12.5, 0.5-15 g/L g/L, 2.5-5 g/L, 2.5-7.5 g/L, 2.5-10 g/L, 2.5-12.5 g/L, 2.5-15 g/L, 5-7.5 g/L, 5-10 g/L, 5-12.5 g/L, 5-15 g/L, 7.5-10 g/L, 7.5-12.5 g/L, 7.5-15 g/L, 10-12.5 g/L, 10-15 g/L, or 12.5-15 g/L.

The internalized HMO backbone precursor, such as lactose, participates in the glycosyltransferase induced glycosylation reaction, in which a glycosyl residue of an activated nucleotide donor produced by the cell is transferred so that the acceptor is glycosylated. Optionally, when more than one glycosyltransferase is/are expressed by the cell, additional glycosylation reactions can occur resulting in the formation of the target oligosaccharide and/or side-products. The cell preferably lacks any enzyme activity which would degrade the oligosaccharide derivatives produced in the cell.

### Harvesting

The term “harvesting” in the context in the invention relates to collecting the produced HMO(s) following the termination of fermentation. In different embodiment(s) it may include collecting the HMO(s) included in both the biomass (i.e., the genetically engineered cell) and cultivation media, i.e., before/without separation of the fermentation broth from the biomass. In other embodiment(s) the produced HMO(s) may be collected separately from the biomass and fermentation broth, i.e., after/following the separation of biomass from cultivation media (i.e., fermentation broth).

The separation of cells from the medium can be carried out with any of the methods well known to the skilled person in the art, such as any suitable type of centrifugation or filtration. The separation of cells from the medium can follow immediately after harvesting the fermentation broth or be carried out at a later stage after storing the fermentation broth at appropriate conditions. Recovery of the produced HMO(s) from the remaining biomass (or total fermentation) include extraction thereof from the biomass (the production cells).

It can be done by any suitable methods of the art, e.g., by sonication, boiling, homogenization, enzymatic lysis using lysozyme, or freezing and grinding.

After recovery from fermentation, HMO(s) are available for further processing and purification.

Use of the Method or the Genetically Engineered Cell for the Production and/or Manufacturing

The disclosure also relates to any commercial use of the method, or the genetically engineered cell described herein.

Thus, in one or more exemplary disclosures(s), the method or genetically engineered cell according to the invention is used in the manufacturing of one or more HMOs. The one or more HMOs can be selected from the group consisting of LNT-II, pLNnH, LNT, LNnT, LNFP-I, LNFP-II, LNFP-III, LNFP-V, LNFP-VI, LNDFH-I, LNDFH-II, LNDFH-III, 2′-FL, DFL, 3-FL, LST-a, 3′-SL, 6′-SL, LST-b, LST-c, FSL, FLST-a, DSLNT, LNnH and LNH. In a presently preferred embodiment, the one or more HMOs has an LNT-II core such as one or more HMO(s) is selected from the group consisting of LNT, LNnT and LNFP-I.

In one or more exemplary embodiment(s), the method and/or the genetically engineered cell is used in the manufacturing of more than one HMO(s), wherein the one or more HMOs is/are selected from the group consisting of LNT, LNnT and LNFP-I.

In one or more further exemplary embodiment(s), the method and/or the genetically engineered cell according to the present disclosure, is used in the manufacturing of more than one HMO(s), wherein the HMOs are LNT and/or LNnT.

### Manufacturing of HMOs

To produce one or more HMOs, the genetically engineered cells as described herein are cultivated according to the procedures known in the art in the presence of a suitable carbon and energy source, e.g., glucose, glycerol or sucrose, and a suitable acceptor, e.g., lactose or any HMO, and the produced HMO blend is harvested from the cultivation media and the microbial biomass formed during the cultivation process. Thereafter, the HMOs are purified according to the procedures known in the art, e.g., such as described in WO2016095924, WO2015188834, WO2017152918, WO2017182965, US20190119314, and the purified HMOs are used as nutraceuticals, pharmaceuticals, or for any other purpose, e.g., for research.

Manufacturing of HMOs is typically accomplished by performing cultivation in larger volumes. The term “manufacturing” and “manufacturing scale” in the meaning of the invention defines a fermentation with a minimum volume of 5 L culture broth. Usually, a “manufacturing scale” process is defined by being capable of processing large volumes of a preparation containing the product of interest and yielding amounts of the HMO product of interest that meet, e.g., in the case of a therapeutic compound or composition, the demands for clinical trials as well as for market supply. In addition to the large volume, a manufacturing scale method, as opposed to simple lab scale methods like shake flask cultivation, is characterized by the use of the technical system of a bioreactor (fermenter) which is equipped with devices for agitation, aeration, nutrient feeding, monitoring and control of process parameters (pH, temperature, dissolved oxygen tension, back pressure, etc.). To a large extent, the behavior of an expression system in a lab scale method, such as shake flasks, benchtop bioreactors or the deep well format described in the examples of the disclosure, does allow to predict the behavior of that system in the complex environment of a bioreactor.

### Manufactured Product

The term “manufactured product” according to the use of the genetically engineered cell or the nucleic acid construct refer to the one or more HMOs indented as the one or more product HMO. The various products are described above.

Advantageously, the methods disclosed herein provides both a decreased ratio of by-product to product and an increased overall yield of the product (and/or HMOs in total). This, less by-product formation in relation to product formation facilitates an elevated product production and increases efficiency of both the production and product recovery process, providing superior manufacturing procedure of HMOs.

The manufactured product may be a powder, a composition, a suspension, or a gel comprising one or more HMOs.

### General

The terms Lacto-N-triose, LNT-II, LNT II, LNT2 and LNT 2, are used interchangeably.

### BRIEF DESCRIPTION OF THE FIGURES

FIG. 1 The effect of lacY over-expression on the relative titers of LNT-II, LNT, pLNH2 and the total HMO content for (a) the yberC-expressing strains MP1 and MP2 and (b) the nec-expressing strains MP3 and MP4, as revealed by the analysis of total samples.

FIG. 2 The effect of lacY over-expression on (a) the LNT fraction in % of the final HMO blend and (b) the final optical density reached by the yberC-expressing strains MP1 and MP2 and the nec-expressing strains MP3 and MP4.

FIG. 3 The effect of lacY over-expression on the fraction of LNT-II, LNT and pLNH2 detected in the supernatant of cultures of (a) the yberC-expressing strains MP1 and MP2 and (b) the nec-expressing strains MP3 and MP4, as revealed by the analysis of supernatant and cell pellet samples.

FIG. 4 The effect of lacY over-expression on the relative titers of LNT-II, LNT, pLNnH and the total HMO content for the vag-expressing strains MP5 and MP6, as revealed by the analysis of total samples.

FIG. 5 The effect of lacY over-expression on (a) the LNnT fraction in % of the final HMO blend and (b) the final optical density reached by the vag-expressing strains MP5 and MP6.

FIG. 6 Performance of LNT producing strains MP7 and MP8 in fermentation runs using a high lactose process. Time course profiles shown for: (a) lactose concentration, (b) biomass concentration, (c) relative accumulated LNT yields on sucrose, (d) ratio of LNT-II to LNT, and (e) ratio of pLNH2 to LNT.

FIG. 7 Performance of LNnT producing strains MP5 and MP6 in fermentation runs using a high lactose process. Time course profiles shown for: (a) lactose concentration, (b) biomass concentration, (c) relative accumulated LNnT yields on sucrose, (d) ratio of LNT-II to LNnT, and (e) ratio of pLNnH to LNnT.

FIG. 8 3′SL production as % of the respective MFS transporter strain set to 100%. Strains containing the MFS transporter nec are presented as the dotted bars, yberC strains are cross stiped and fred strains are horizontally striped.

FIG. 9 2′FL production as % of the control nec transporter strain (MP18) set to 100%. The 2′FL titers of strains containing the MFS transporter nec in combination with an additional copy of lacY expressed from either PglpF_SD7 (MP19) or PglpF (MP20) are shown relative to the nec transporter strain.

### SEQUENCE ID'S

The current application contains a sequence listing in text format and electronical format which is hereby incorporated by reference. Table 9 provides a summary of the sequences in the present application.

TABLE 9
sequences

| Protein/gene/promoter | | | |
|---|---|---|---|
| Abbreviation | Function | Public reference | SEQ ID NO: |
| LacY | Lactose permease | NP_414877.1 | 1 |
| | | WP_042094275.1 | 3 |
| | | WP_000291549.1 | 4 |
| | | WP_089607162.1 | 5 |
| | | WP_152280604.1 | 6 |
| | | EGT4952364.1 | 7 |
| | | WP_134216118.1 | 8 |
| | | EDI1749185.1 | 9 |
| | | WP_084912833.1 | 10 |
| | | WP_103826752.1 | 11 |
| | | WP_021804673.1 | 12 |
| | | WP_084984472.1 | 13 |
| | | WP_199428647.1 | 14 |
| | | WP_046596210.1 | 15 |
| | | XP_452193.1 | 16 |
| lacY | E. Coli K-12 lactose | NC_000913.3 | 2 |
| | permease gene | | |
| CvB3galT | β-1,3-Gal-transferase | WP_080969100.1 | 17 |
| GalTK | β-1,3-Gal-transferase | homologous to BD182026.1 | 18 |
| GalT | β-1,4-gal-transferase | WP_001262061.1 | 19 |
| LgtA | β-1,3-GlcNAc- | WP_033911473.1 | 20 |
| | transferase | | |
| PmnagT | β-1,3-GlcNAc- | WP_014390683.1 | 21 |
| | transferase | | |
| HD0466 | β-1,3-GlcNAc- | WP_010944479.1 | 22 |
| | transferase | | |
| FutC | α-1,2-fucosyl-transferase | WP_080473865.1 (with two | 23 |
| | | additional amino acids (LG) | |
| | | at the C-terminus) | |
| Smob | α-1,2-fucosyl-transferase | WP_126455392.1 | 24 |
| Mtun | α-1,2-fucosyl-transferase | WP_031437198.1 | 25 |
| FutA | α-1,3-fucosyl-transferase | NP_207177.1 | 26 |
| FucT | α-1,3-fucosyl-transferase | AAB81031.1 | 27 |
| FucTIII | α-1,3-fucosyl-transferase | AAR88243.1 | 28 |
| Pd2 | α-2,3-sialyl-transferase | — | 29 |
| Nst | α-2,3-sialyl-transferase | — | 30 |
| Gmd | GDP-mannose 4,6- | NP_416557.1 | 31 |
| | dehydratas | WP_000048190.1 | 32 |
| WcaG | GDP-L-fucose synthase | NP_416556.1 | 33 |
| | | WP_000043654.1 | 34 |
| WcaH | GDP-mannose | NP_416555.2 | 35 |
| | mannosyl hydrolase | WP_001393539.1 | 36 |
| Wcal | colanic acid biosynthesis | NP_416554.1 | 37 |
| | fucosyltransferase Wcal | WP_000699693.1 | 38 |
| ManC | mannose-1-phosphate | NP_416553.1 | 39 |
| | guanylyltransferase | WP_000079274.1 | 40 |
| ManB | Phosphomannomutase | NP_416552.1 | 41 |
| | | WP_001350528.1 | 42 |
| Pgm | Phosphoglucomutase | NP_415214.1 | 43 |
| | | WP_001396326.1 | 44 |
| GalU | UTP-glucose-1- | NP_415752.1 | 45 |
| | phosphate | WP_000718995.1 | 46 |
| | uridylyltransferase | | |
| GalE | UDP-glucose 4- | NP_415280.3 | 47 |
| | epimerase | WP_001265438.1 | 48 |
| GlmM | Phosphoglucosamine | NP_417643.1 | 49 |
| | mutase | WP_000071134.1 | 50 |
| GlmU | N-acetylglucosamine-1- | NP_418186.1 | 51 |
| | phosphate | WP_000933736.1 | 52 |
| | uridyltransferase and | | |
| | glucosamine-1- | | |
| | phosphate | | |
| | acetyltransferase | | |
| GlmS | L-glutamine-D-fructose- | NP_418185.1 | 53 |
| | 6-phosphate | WP_000334099.1 | 54 |
| | aminotransferase | | |
| NeuA | CMP-Neu5Ac | AAK91728.1 | 55 |
| | synthetase | WP_006881452.1 | 56 |
| NeuB | Neu5 Ac synthase | AAK91726.1 | 57 |
| | | WP_023580510.1 | 58 |
| NeuC | UDP-GlcNAc 2- | AAK91727.1 | 59 |
| | epimerase | WP_000723250.1 | 60 |
| | | CAR04561.1 | 61 |
| Vag | MFS transporter | WP_048785139.1 | 62 |
| Fred | MFS transporter | WP_087817556.1 | 63 |
| Marc | MFS transporter | WP_060448169.1 | 64 |
| Bad | MFS transporter | WP_017489914.1 | 65 |
| Nec | MFS transporter | WP_092672081.1 | 66 |
| YberC | MFS transporter | EEQ08298.1 | 67 |
| Plac | promoter | — | 68 |
| PgatY_70UTR | promoter | WO2020255054A1 | 69 |
| PglpF | promoter | WO2019123324A1 | 70 |
| PglpF_SD1 | promoter | WO2019123324A1 | 71 |
| PglpF_SD10 | promoter | WO2019123324A1 | 72 |
| PglpF_SD2 | promoter | WO2019123324A1 | 73 |
| PglpF_SD3 | promoter | WO2019123324A1 | 74 |
| PglpF_SD4 | promoter | WO2019123324A1 | 75 |
| PglpF_SD5 | promoter | WO2019123324A1 | 76 |
| PglpF_SD6 | promoter | WO2019123324A1 | 77 |
| PglpF_SD7 | promoter | WO2019123324A1 | 78 |
| PglpF_SD8 | promoter | WO2019123324A1 | 79 |
| PglpF_SD9 | promoter | WO2019123324A1 | 80 |
| Plac_16UTR | promoter | WO2020255054A1 | 81 |
| PmglB_70UTR | promoter | WO2020255054A1 | 82 |
| PmglB_70UTR_SD4 | promoter | WO2020255054A1 | 83 |
| CP6 | promoter | — | 84 |
| PosmY | promoter | — | 85 |
| SacC_AgaI | glycoside hydrolase | WP_103853210.1 | 86 |
| | family 32 protein | | |
| Bff | beta-fructofuranosidase | BAD18121.1 | 87 |
| | protein | | |
| scrY | Sucrose porin | WO2015197082 | 88 |
| scrA | PTS system sucrose- | WO2015197082 | 89 |
| | specific EIIBC | | |
| | component | | |
| scrB | Sucrose-6-phosphate | WO2015197082 | 90 |
| | hydrolase | | |
| scrR | Sucrose operon | WO2015197082 | 91 |
| | repressor | | |
| glpR | Regulator of the Glp | | 92 |
| | Operon | | |
| PmglB_70UTR_SD8 | promoter | WO2020255054 | 93 |
| PmglB_70UTR_SD10 | promoter | WO2020255054 | 94 |
| PmglB_54UTR | promoter | WO2020255054 | 95 |
| Plac_70UTR | promoter | WO2019123324 | 96 |
| PmglB_70UTR_SD9 | promoter | WO2020255054 | 97 |
| PmglB_70UTR_SD5 | promoter | WO2020255054 | 98 |
| PmglB_70UTR_SD7 | promoter | WO2020255054 | 99 |
| PglpA_70UTR | promoter | WO2019123324 | 100 |
| PglpT_70UTR | promoter | WO2019123324 | 101 |
| PmglB_16UTR | promoter | WO2020255054 | 102 |
| PglpA_16UTR | promoter | WO2019123324 | 103 |

### EXAMPLES

### Example 1—Improvement of LNT Production Systems by Increasing the Expression of the lacY Gene Encoding Lactose Permease in Cells Expressing a Heterologous MFS Transporter

### Genotype of Strains MP1, MP2, MP3 and MP4

The strains (genetically engineered cells) constructed in the present application were based on Escherichia coli K-12 DH1 with the genotype: F⁻, λ⁻, gyrA96, recA1, relA1, endA1, thi-1, hsdR17, supE44. Additional modifications were made to the E. coli K-12 DH1 strain to generate the platform strain “MDO” with the following modifications: lacZ: deletion of 1.5 kbp, lacA: deletion of 0.5 kbp, nanKETA: deletion of 3.3 kbp, melA: deletion of 0.9 kbp, wcaJ: deletion of 0.5 kbp, mdoH: deletion of 0.5 kbp, and insertion of Plac promoter upstream of the gmd gene.

Methods of inserting or deleting gene(s) of interest into the genome of E. coli are well known to the person skilled in the art. Insertion of genetic cassettes into the E. coli chromosome can be done using gene gorging (see e.g., Herring and Blattner 2004 J. Bacteriol. 186: 2673-81 and Warming et al 2005 Nucleic Acids Res. 33(4): e36) with specific selection marker genes and screening methods.

Based on the platform strain “MDO” (e.g., also reported in WO2020255054A1 or WO2019123324A1), the modifications summarised in the table below, were made to obtain the fully chromosomal strains MP1, MP2, MP3 and MP4. The strains can produce the tetrasaccharide HMO LNT. The glycosyltransferase enzymes LgtA (a beta-1,3-N-acetyloglucosamine transferase) from N. meningitidis and GalTK (a beta-1,3-galactosyltransferase) from H. pylori are present in all four strains. Moreover, MP1 and MP2 express the heterologous transporter YberC from Yersinia bercovieri, while the strains MP3 and MP4 express the heterologous transporter Nec from Rosenbergiella nectarea. Moreover, the strains MP2 and MP4 over-express the lacY gene from an additional PglpF-driven genomic copy, while the strains MP1 and MP3 do not.

In the present example, it is demonstrated how the over-expression of the lacY gene coding lactose permease is used as a genetic tool to enhance LNT production in strains that already express the heterologous transporter YberC or Nec. This invention also demonstrates how the over-expression of the lacY gene can be advantageously used to increase the total HMO content of the broth, and simultaneously reduce the formation of other HMOs, such as LNT-II, and increase the LNT content in the final HMO blend. As shown in table 2, the only difference among each strain of the two strain pairs, namely MP1-MP2 and MP3-MP4, is the presence of an additional lacY expression cassette at a genomic locus that is different from the native lacY locus.

TABLE 2
Genotypes of the strains MP1, MP2, MP3 and MP4

| Strain ID | Genotype | Plasmid-free |
|---|---|---|
| MP1 | MDO x3 GlcNACT* x2 GalTK**, Plac-yberC | LNT |
| MP2 | MDO x3 GlcNACT* x2 GalTK**, | LNT |
| | Plac-yberC, PglpF-lacY | |
| MP3 | MDO x3 GlcNACT* x2 GalTK**, PglpF-nec | LNT |
| MP4 | MDO x3 GlcNACT* x2 GalTK**, | LNT |
| | PglpF-nec, PglpF-lacY | |

*GlcNAcT: beta-1,3-N-acetyloglucosamine transferase
**GalTK: beta-1,3-galactosyltransferase

### Deep Well Assay

The strains disclosed in the present example were screened in 96 deep well plates using a 4-day protocol. During the first 24 hours, precultures were grown to high densities and subsequently transferred to a medium that allowed induction of gene expression and product formation. More specifically, during day 1, fresh precultures were prepared using a basal minimal medium (BMM) supplemented with magnesium sulphate, thiamine and glucose. The precultures were incubated for 24 hours at 34° C. and 1000 rpm shaking and then further transferred to a new BMM (pH 7.5) in order to start the main culture. The new BMM was supplemented with magnesium sulphate, thiamine, a bolus of 20% glucose solution (0.5 μL per mL) and a bolus of 20% lactose solution (0.1 μL per μL).

Moreover, a 20% stock solution of a specific polysaccharide was provided as carbon source, accompanied by the addition of a specific hydrolytic enzyme, so that glucose was released at a rate suitable for carbon-limited growth and similar to that of a typical fed-batch fermentation process. The main cultures were incubated for 72 hours at 28° C. and 1000 rpm shaking. For the analysis of total broth, the 96 well plates were boiled at 100° C., subsequently centrifuged, and finally the supernatants were analyzed by HPLC.

### Results

Strains were characterized in deep well assays and samples were collected from the total broth, the supernatant, and the cell pellet. All samples were analysed for HMO content by HPLC following the 72-hour protocol described above.

The concentration of the detected HMOs (in g/L) in each sample was used to calculate the % quantitative differences in the HMO content of the strains tested, i.e., the % differences in the HMO concentrations of lacY-expressing cells relative to the ones expressing lacY at physiological levels. Moreover, the absolute fraction (%) of LNT in the final HMO blend was calculated by considering the HMO concentrations detected by HPLC, i.e., LNT-II and LNT concentrations. The final optical density at 600 nm was also measured for all strains at the end of the experiment, i.e., after 72 hours in the production phase. Finally, the HPLC measurements for the supernatant and pellet samples were used to calculate the absolute sugar ratio (%) of the supernatant (S) fraction to the sum of the supernatant and pellet fractions (total, T).

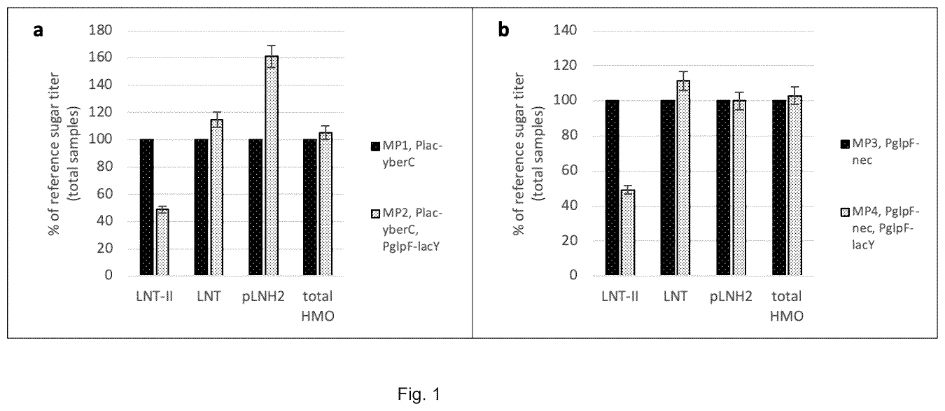

As revealed by the analysis of total samples in deep-well cultures, some gains in LNT and total HMO titers can be obtained by over-expressing the lacY gene both in nec- and yberC-expressing cells. Specifically, the strain expressing the Nec transporter and over-expressing the lacY gene, MP2, produced approximately 15% more LNT and provided approximately 10% more total HMO content than the nec-expressing strain MP1 that has wild-type expression levels of the lacY gene (FIG. 1a). Similarly, gains up to 15% both in LNT titers and the total HMO content can be obtained by over-expressing the lacY gene in the yberC-expressing strain MP3 to generate the strain MP4748 (FIG. 1b). Moreover, the increase in the intracellular lactose concentration mediated by lacY over-expression in both nec- and yberC-expressing cells (strains MP2 and MP4) results in markedly lower by-product (LNT-II) formation compared to cells that express lacY at physiological levels (strains MP1 and MP3) (FIGS. 1a-1b).

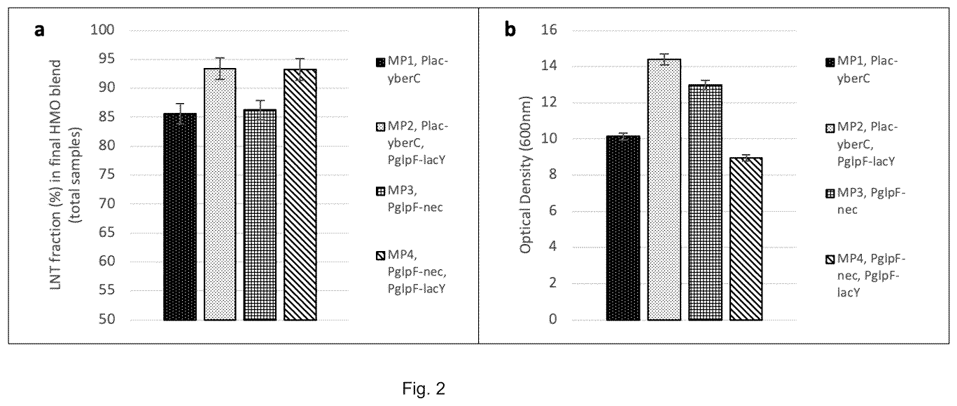

The facts mentioned above are directly reflected to the LNT content (%) in the final HMO blend of both nec- and yberC-expressing cells that over-express the lacY gene. Specifically, the LNT fraction of the final HMO blend generated by cells over-expressing the lacY and nec genes (strain MP3) or the lacY and yberC genes (strain MP2) is approximately 10% higher than for cells that express the nec (strain MP4) or yberC (strain MP1) gene alone (FIG. 2a). Interestingly, the effect of lacY over-expression on biomass formation for heterologous MFS-expressing strains can vary depending on the heterologous MFS being expressed. In detail, the strain MP4 that over-expresses both the lacY and nec genes reaches significantly lower optical density values after 72 hours of cultivation compared to the similar strain MP3 that does not over-express the lacY gene (FIG. 2b). The opposite is true for the strain MP2 that over-expresses both the lacY and yberC genes, i.e., MP2 forms higher biomass than the strain MP1 that expresses the yberC gene alone (FIG. 2b)

In conclusion, the over-expression of the lacY gene in transporter expressing LNT production strains seems to change the sugar transport dynamics across the cell membrane in such a manner that the final LNT and total HMO titer in these strains is much higher when the lacY gene is over-expressed (strains MP2 and MP4) than when it is expressed at physiological levels (strains MP1 and MP3).

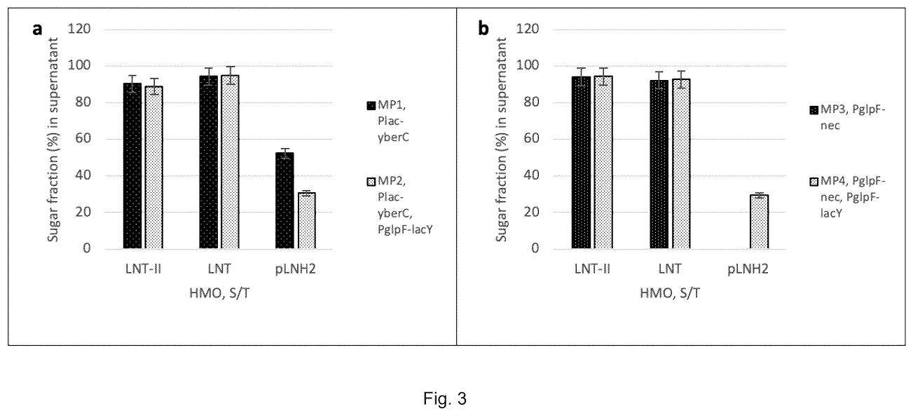

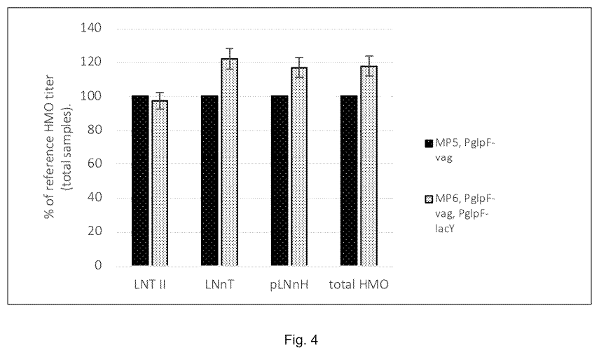

As it is apparent from the analysis of the supernatant and pellet fractions of the broth, the % fraction of LNT and LNT-II in the supernatant is not affected by the over-expression of the lacY gene in yberC-expressing cells (FIG. 3a). Approximately 25% less pLNH2 is, however, detected in the culture medium when yberC-expressing cells over-express lacY (strain MP2) rather than when lacY is solely expressed from the Plac promoter of the lac operon of E. coli (strain MP1) (FIG. 3a).

As for yberC-expressing cells, the fraction of LNT and LNT-II detected in the supernatant is not affected by the over-expression of the lacY gene in nec-expressing cells (FIG. 3b). Contrary to yberC-expressing cells (strain MP3), however, the over-expression of the lacY gene in nec-expressing cells (strain MP4) results in higher pLNH2 in the culture medium (FIG. 3b).

### Example 2—Improvement of LNnT Production Systems by Increasing the Expression of the lacY Gene Encoding Lactose Permease in Cells Expressing a Heterologous MFS Transporter

### Genotype of Strains MP5 and MP6

Based on the platform strain “MDO” (e.g., see example 1 and also reported in WO2020255054A1 or WO2019123324A1), the modifications summarised in the table below, were made to obtain the fully chromosomal strains MP5 and MP6. The strains can produce the tetrasaccharide HMO LNnT. The glycosyltransferase enzymes LgtA (a beta-1,3-N-acetyloglucosamine transferase) from N. meningitidis and GalT (a beta-1,4-galactosyltransferase) from H. pylori are present in both strains. Moreover, MP5 and MP6 express the heterologous transporter Vag from Pantoea vagans, and the strain MP6, but not MP5, over-expresses the lacY gene from an additional genomic copy under the control of the PglpF promoter.

In the present Example, it is demonstrated how the over-expression of the lacY gene coding lactose permease is used as a genetic tool to enhance LNnT production in strains that already express the heterologous transporter Vag. This invention also demonstrates how the over-expression of the lacY gene can be advantageously used to increase the pLNnH and total HMO content of the broth. As shown in table 3 below, the only difference between the two vag-expressing strains, MP5 and MP6, is the presence of an additional lacY expression cassette at a genomic locus that is different from the native lacY locus.

TABLE 3
Genotypes of the strains MP5 and MP6

| Strain ID | Genotype | Product |
|---|---|---|
| MP5 | MDO x2 GlcNACT* x1 GalT**, | LNnT |
| | PglpF-vag, scrBRYA*** | |
| MP6 | MDO x2 GlcNACT* x1 GalT**, PglpF-vag, | LNnT |
| | PglpF-lacY, scrBRYA*** | |

*GlcNACT: beta-1,3-N-acetyloglucosamine transferase
**GalT: beta-1,4-galactosyltransferase
***scrBRYA: sucrose utilization genes

### Deep Well Assay

The strains disclosed in the present example were screened in 96 deep well plates using a 4-day protocol. During the first 24 hours, precultures were grown to high densities and subsequently transferred to a medium that allowed induction of gene expression and product formation. More specifically, during day 1, fresh precultures were prepared using a basal minimal medium (BMM) supplemented with magnesium sulphate, thiamine and glucose. The precultures were incubated for 24 hours at 34° C. and 1000 rpm shaking and then further transferred to a new BMM (pH 7.5) in order to start the main culture. The new BMM was supplemented with magnesium sulphate, thiamine, a bolus of 20% glucose solution (0.5 μL per mL) and a bolus of 20% lactose solution (0.1 μL per μL).

Moreover, a 20% stock solution of a specific polysaccharide was provided as carbon source, accompanied by the addition of a specific hydrolytic enzyme, so that glucose was released at a rate suitable for carbon-limited growth and similar to that of a typical fed-batch fermentation process. The main cultures were incubated for 72 hours at 28° C. and 1000 rpm shaking. For the analysis of total broth, the 96 well plates were boiled at 100° C., subsequently centrifuged, and finally the supernatants were analysed by HPLC.

### Results

Strains were characterized in deep well assays and samples were collected from the total broth and analysed for HMO content by HPLC following the 72-hour protocol described above. The concentration of the detected HMOs (in g/L) in each sample was used to calculate the % quantitative differences in the HMO content of the strains tested, i.e., the % differences in the HMO concentrations of lacY-expressing cells (strain MP6) relative to the ones expressing lacY at physiological levels (strain MP5). Moreover, the absolute fraction (%) of LNnT in the final HMO blend was calculated by considering the HMO concentrations detected by HPLC, i.e., LNT-II, LNnT and pLNnH concentrations. The final optical density at 600 nm was also measured for all strains at the end of the experiment, i.e., after 72 hours in the production phase.

As revealed by the analysis of total samples in deep-well cultures, marked gains in LNnT, pLNnH and total HMO titers can be obtained by over-expressing the lacY gene in vag-expressing cells. Specifically, the strain expressing the Vag transporter and over-expressing the lacY gene, MP6, produced approximately 20% more LNnT, 20% more pLNnH and had 20% more total HMO content than the vag-expressing strain MP5 that expresses the lacY gene at wild-type levels (FIG. 4). No significant differences in the LNT-II content among the two strains could be detected (FIG. 4).

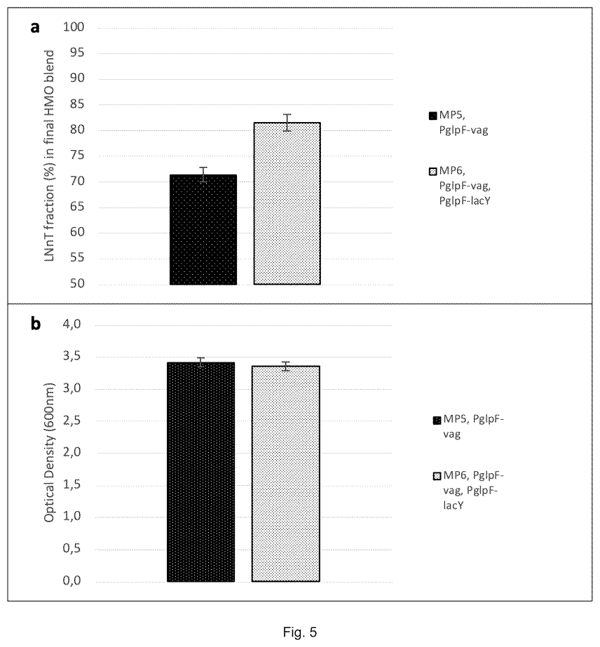

The facts mentioned above are directly reflected to the LNnT content (%) in the final HMO blend of vag-expressing cells that over-express the lacY gene. Specifically, the LNnT fraction of the final HMO blend generated by the strain MP6 that over-expresses the lacY gene and the vag gene is approximately 10% higher than for the strain MP5 that expresses the vag gene alone (FIG. 5a). Contrary to the marked impact of lacY over-expression on biomass formation for the LNT production strains presented in example 1, the impact of lacY over-expression on biomass formation for the heterologous MFS-expressing LNnT strain MP6 is negligible. In detail, the final optical density of cells over-expressing both the lacY and vag genes (strain MP6) is very similar to the one of cells expressing the vag gene alone (strain MP5) (FIG. 5b).

### Example 3—Fermentation Performance of LNT and LNnT Strains Over-Expressing the lacY Gene and Expressing a Gene Encoding a Heterologous Sugar Exporter

### Genotype of Strains MP7, MP8, MP5 and MP6

Based platform strain “MDO” (e.g., see Example 1 and also reported in WO2020255054A1 or WO2019123324A1), the modifications summarised in table 4, were made to obtain the fully chromosomal strains MP7, MP8, MP5 and MP6. The strains can produce the tetrasaccharide HMO LNT (MP7 and MP8) or LNnT (MP5 and MP6). All four strains can grow on sucrose. The glycosyltransferase enzymes LgtA (a beta-1,3-N-acetyloglucosamine transferase) from N. meningitidis is present in all four strains, while the GalTK (a beta-1,3-galactosyltransferase) or the GalT (a beta-1,4-galactosyltransferase) from H. pylori is introduced in strains MP7-MP8 and MP5-MP6, respectively. Moreover, the strains MP7 and MP8 express the heterologous transporter YberC from Yersinia bercovieri, while the strains MP5 and MP6 express the heterologous transporter Vag from Pantoea vagans. Moreover, the strains MP8 and MP6 over-express the lacY gene from an additional PglpF-driven genomic copy, while the strains MP7 and MP5 do not.

In the present Example, it is demonstrated how the over-expression of the lacY gene coding lactose permease is used as a strain engineering tool to enhance LNT or LNnT production in fed-batch fermentations using strains that already express the heterologous transporter YberC or Vag, respectively. This invention also demonstrates how the over-expression of the lacY gene can be advantageously used to obtain higher total HMO content in the fermentation broth, and simultaneously modulate the formation of HMOs other than LNT and LNnT, e.g., LNT-II, pLNnH and pLNH2. As shown in table 4, the only difference between the two pairs of strains, MP7-MP8 and MP5-MP6, is the presence of an additional lacY expression cassette at a genomic locus that is different from the native lacY locus.

TABLE 4
Genotypes of the strains MP7, MP8, MP5 and MP6

| Strain ID | Genotype | Product |
|---|---|---|
| MP7 | MDO x3 GlcNACT* x2 GalTK**, | LNT |
| | Plac-yberC, scrBRYA**** | |
| MP8 | MDO x3 GlcNACT* x2 GalTK**, Plac-yberC, | LNT |
| | PglpF-lacY, scrBRYA**** | |
| MP5 | MDO x2 GlcNACT* x1 GalT***, | LNnT |
| | PglpF-vag, scrBRYA**** | |
| MP6 | MDO x2 GlcNAcT* x1 GalT***, PglpF-vag, | LNnT |
| | PglpF-lacY, scrBRYA**** | |

*GlcNAcT: beta-1,3-N-acetyloglucosamine transferase
**GalTK: beta-1,3-galactosyltransferase
***GalT: beta-1,4-galactosyltransferase
****scrBRYA: sucrose utilization genes

### Fermentation Processes

The fermentations were carried out in 200 mL DasBox bioreactors (Eppendorf, Germany), starting with 100 mL of defined mineral culture medium, consisting of a suitable concentration of a carbon source (sucrose or glucose), MgSO4×7H2O, KOH, NaOH, NH4H2PO4, KH2PO4, trace metal solution, citric acid, antifoam and thiamine. The trace metal solution (TMS) contained Mn, Cu, Fe, Zn as sulfate salts and citric acid. Fermentations were started by inoculation with 2% (v/v) of pre-cultures grown in a defined minimal medium. After depletion of the carbon source present in the batch medium, a sterile feed solution containing sucrose (or glucose), NH₄SO₄, and TMS was fed continuously in a carbon-limited manner using a predetermined, linear profile.

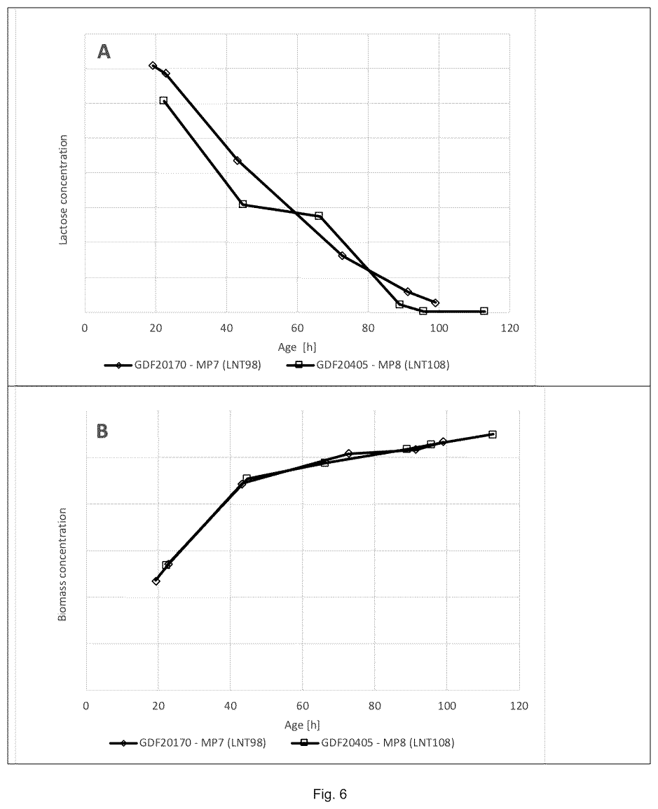

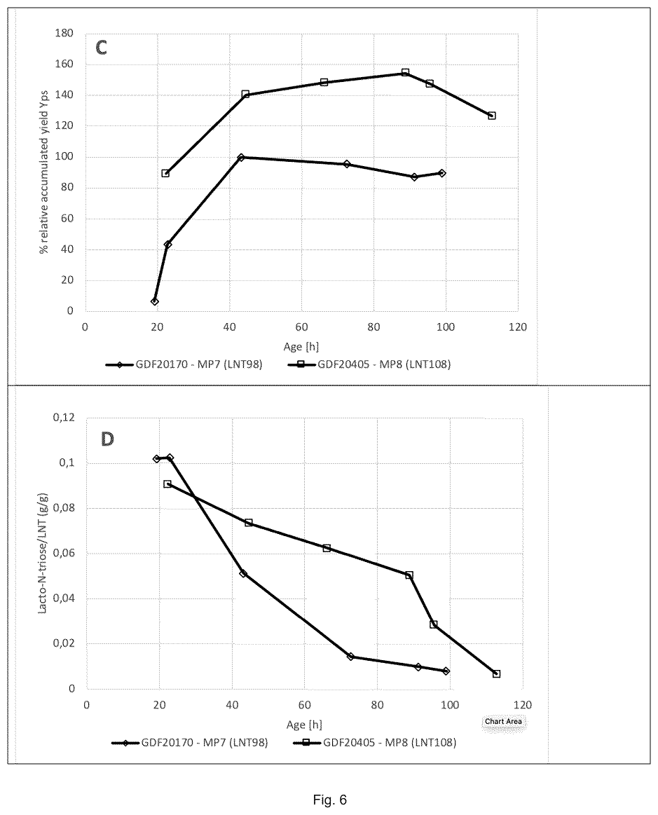

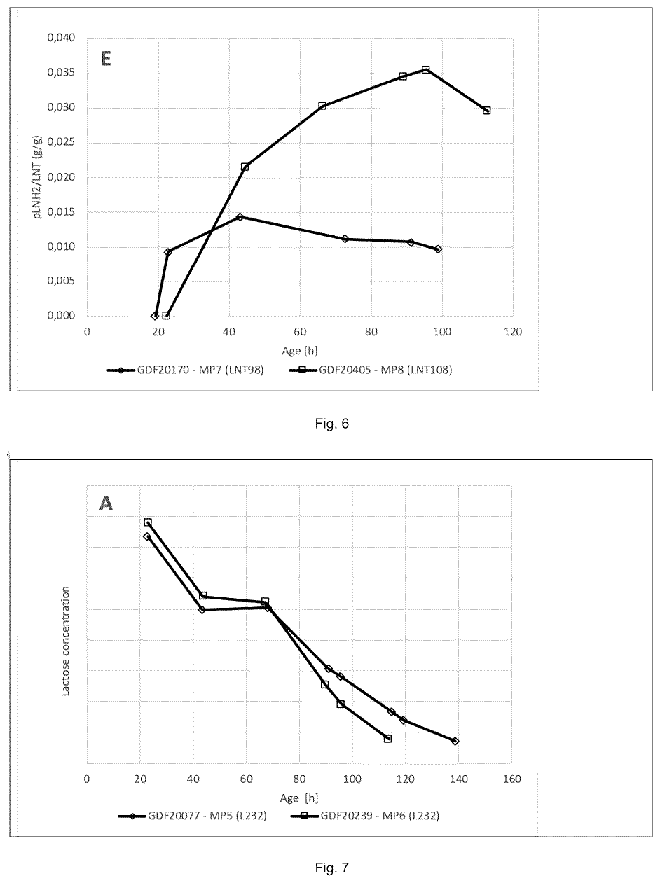

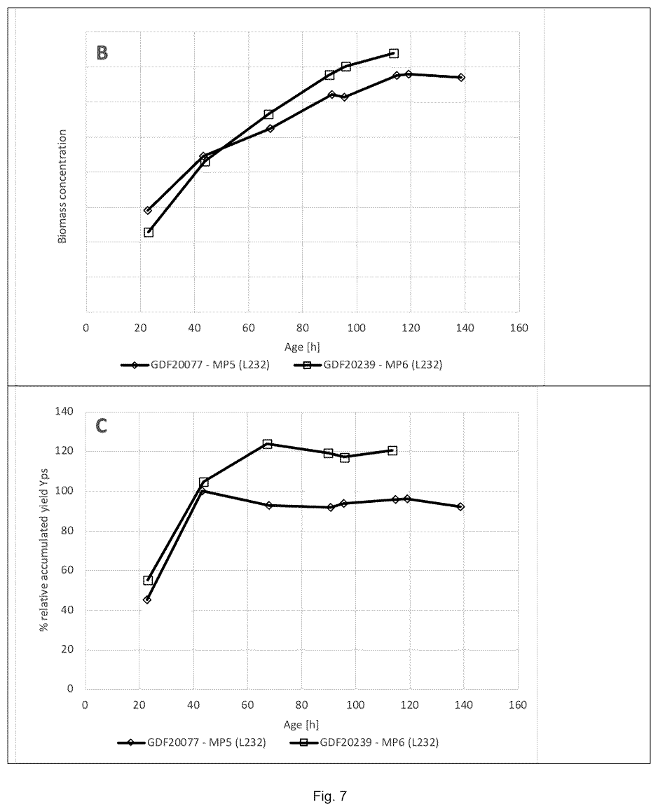

Lactose addition was done by a “high” lactose process (“LNT98” and “LNT108” for LNT fermentations, and “L232” for LNnT fermentations), where lactose monohydrate solution was added by two bolus additions, the first one at approx. 10 hours after feed start, the second one at approx. 70 hours EFT. For LNT fermentations, the processes “LNT98” and “LNT108” differ only in the fact that the second lactose pulse was performed approx. 20 h earlier for LNT108 than for LNT98. In this manner, lactose was ensured not to be the limiting factor for HMO formation at least until 90 hours EFT, as shown in FIGS. 6a and 7a. The process L232 is identical with LNT98.

The pH throughout fermentation was controlled at 6.8 by titration with 14% NH4OH solution. Aeration was controlled at 1 vvm using air, and dissolved oxygen was kept above 23% of air saturation, controlled by the stirrer rate. At 15 min after sucrose feed start, the fermentation temperature setpoint was lowered from 33° C. to 28° C. This temperature drop was conducted without a ramp. The total duration of the fermentations was 4-5 days.

Throughout the fermentations, samples were taken to determine the concentration of LNT or LNnT, LNT-II, lactose, pLNnH or pLNH2 and other minor by-products using HPLC. Total broth samples were diluted three-fold in deionized water and boiled for 20 minutes. This was followed by centrifugation at 17000 g for 3 minutes, where after the resulting supernatant was analysed by HPLC. The above measurements were used along with data on carbon source utilization to accurately calculate product yields on sucrose as well as the ratios of LNT-II and hexasaccharide (pLNnH or pLNH2) relative to the main product (respectively, LNnT or LNT).

### Results

All four fermentations ran in a stable manner for at least 4 days (FIGS. 6 and 7).

As shown in FIG. 6c, the performance of the LNT strain that bears an extra copy of the lacY gene (strain MP8) is superior to the one observed by the strain that expresses lacY at physiological levels (strain MP7), with the former showing an approximately 50% higher LNT yield on sucrose (FIG. 6c), while retaining the biomass formation at a comparable level (FIG. 6B). Moreover, as revealed by the calculations of ratios of by-products to LNT, the strains MP8 and MP7 show similar LNT-II levels (FIG. 6d), while the over-expression of the lacY gene in strain MP8 results in increased pLNH2 formation (FIG. 6e).

As shown in FIG. 7c, the performance of the LNnT strain that bears an extra copy of the lacY gene (strain MP6) is superior to the one observed by the strain that expresses lacY at physiological levels (strain MP5), with the former showing an approximately 25% higher LNnT yield on sucrose (FIG. 7c), while retaining the biomass formation at a comparable level (FIG. 7b). As mentioned above for the LNT strains, the strains MP8 and MP7 show similar LNT-II levels (FIG. 7d), and the over-expression of the lacY gene in strain MP6 results in higher pLNnH formation than in strain MP5 (FIG. 7e).

### Example 4—Increased Expression of lacY in Combination with an MFS Transporter does not Improve 3′SL Expression

In Examples 1 to 3 it has been illustrated that LacY overexpression in combination with certain MFS transporters has a positive effect on LNT and LNnT production.

In the following example the MFS transporters nec, yberC or fred (1 copy genomically integrated) have been tested in a 3′-SL producing strain without (MP9, MP12 and MP15) or with overexpression of LacY, either as an extra copy from the chromosome (MP10, MP13 and MP16) or from a medium copy nr plasmid (pSU2719-lacY-chr) (MP11, MP14 and MP17).

All strains were based on the platform strain “MDO” (e.g., see example 1 and also reported in WO2020255054A1 or WO2019123324A1) and contained a truncated version (29 aa n-terminal deletion) of the α-2,3-sialyltransferase from Neisseria meningitidis, nst (GenBank assession nr. AAC44541.1), heterologous CMP-Neu5Ac synthetase, neuA (GenBank assession nr. AAK91728.1), heterologous sialic acid synthase, neuB (GenBank assession nr. AAK91726.1) and heterologous GlcNAc-6-phosphate 2 epimerase, neuC (GenBank assession nr. AAK91727.1) incorporated with a single copy at different loci in the genome of the MDO strain. The genotypes of the tested strains are given in table 5.

TABLE 5
Genotypes of the strains 3′-SL producing strains with different MFS transporters

| Strain ID | Genotype | Transporter |
|---|---|---|
| MP9 | MDO x1 PglpF-nst, PglpF-nec, 1xPglpF-neuB, 1xPglpF- | nec |
| | neuC, 1xPglpF-neuA | |
| MP10 | MDO x1 PglpF-nst, PglpF-nec, 1xPglpF-neuB, 1xPglpF- | nec |
| | neuC, 1xPglpF-neuA, x1 PglpF-LacY | |
| MP11 | MDO x1 PglpF-nst, PglpF-nec, 1xPglpF-neuB, 1xPglpF- | nec |
| | neuC, 1xPglpF-neuA, pSU2719-lacY-chr | |
| MP12 | MDO x1 PglpF-nst, PglpF-yberC, 1xPglpF-neuB, 1xPglpF- | yberC |
| | neuC, 1xPglpF-neuA | |
| MP13 | MDO x1 PglpF-nst, PglpF-yberC, 1xPglpF-neuB, 1xPglpF- | yberC |
| | neuC, 1xPglpF-neuA, x1 PglpF-LacY | |
| MP14 | MDO x1 PglpF-nst, PglpF-yberC, 1xPglpF-neuB, 1xPglpF- | yberC |
| | neuC, 1xPglpF-neuA, pSU2719-lacY-chr | |
| MP15 | MDO x1 PglpF-nst, PglpF-fred, 1xPglpF-neuB, 1xPglpF- | fred |
| | neuC, 1xPglpF-neuA, x1 PglpF-LacY | |
| MP16 | MDO x1 PglpF-nst, PglpF-fred, 1xPglpF-neuB, 1xPglpF- | fred |
| | neuC, 1xPglpF-neuA, | |
| MP17 | MDO x1 PglpF-nst, PglpF-fred, 1xPglpF-neuB, 1xPglpF- | fred |
| | neuC, 1xPglpF-neuA, pSU2719-lacY-chr | |

### Deep-Well Assay

The strains disclosed in the present example were screened for 3′SL production in a 96-deep well plate assay. Three replicates per strain were tested. More specifically, during day 1, fresh precultures were prepared using a basal minimal medium (BMM) supplemented with magnesium sulphate, thiamine and glucose. The precultures were incubated at 34° C. with 1000 rpm for 24 h and then transferred to a new BMM (pH 7.5) in order to start the main culture. The new BMM was supplemented with magnesium sulphate, thiamine, a bolus of 20% glucose solution (0.5 μL/mL) and a bolus of 20% lactose solution (0.1 μL/μL).

To the main culture 50% sucrose solution as carbon source (52.5 μl/ml) was provided to the cells accompanied by the addition of sucrose hydrolase (invertase 0.1 g/L) and 50 mg/ml IPTG, so that glucose was released at a rate suitable for C-limited growth allowing for gene expression and 3′SL production. Antibiotic (chloramphenicol 20 mg/ml) was added in wells when required for plasmid maintenance. The main cultures were incubated at 34° C. with 1000 rpm for 96 h. For the analysis of total broth, the 96 well plates were boiled at 100° C., subsequently centrifuged, and finally the supernatants were analysed by HPLC.

### Results

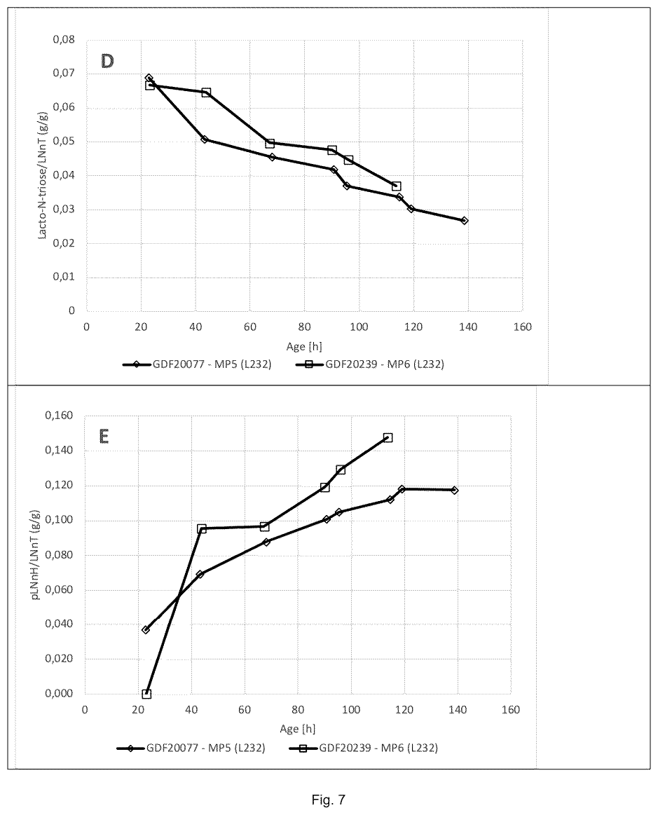

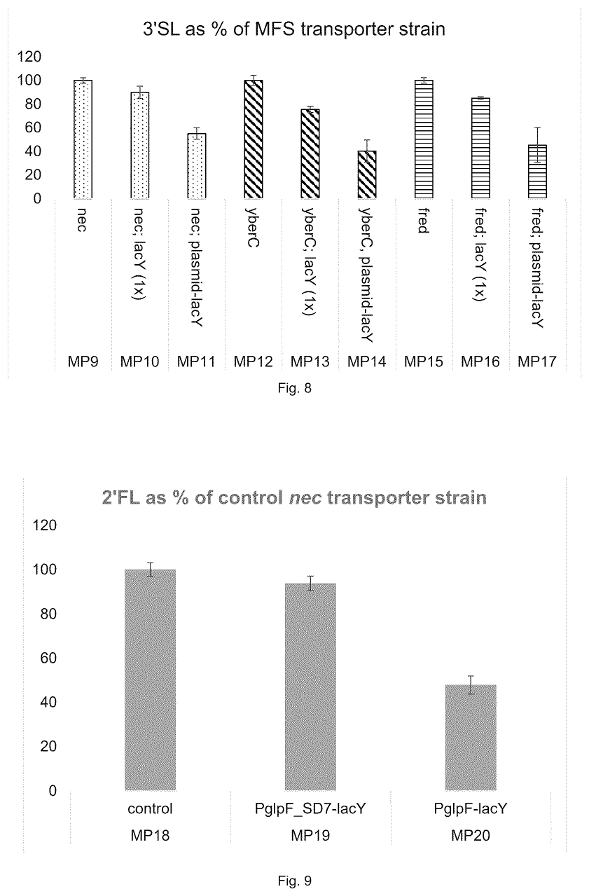

The strains of the present example only produce 3′SL. The results from deep-well assays are shown in FIG. 8. The 3′SL production for the strain expressing the MFS transporter was set to 100 and the corresponding strains with either one extra copy of LacY on the chromosome or LacY expressed from a multicopy plasmid was normalized to the respective MFS transporter strain.

From FIG. 8 it can be seen that in a 3′SL producing strain, overexpression of LacY does not increase the total 3′SL production. In fact, the production seems to be reduced with increased overexpression of lacY as indicated by the strains expressing lacY from a medium copy nr plasmid (MP11, MP14 and MP17, respectively) which have further reduced 3′SL levels compared to strain with a single additional copy of LacY (MP10, MP13 and MP16, respectively).

### Example 5—Increased Expression of lacY in Combination with an MFS Transporter does not Improve 2 FL Expression

In Examples 1 to 3 it has been illustrated that lacY overexpression in combination with certain MFS transports has a positive effect on LNT and LNnT production.

In the following example the MFS transporter Nec (1 copy genomically integrated) was tested in 2′FL producing strains with (MP19 and MP20) or without (MP18) overexpression of lacY. In the strains overexpressing lacy the additional genomic copy was placed under control of a weak promoter (MP19) or a strong promoter (MP20) to assess if the lacy expression level affected the 2′FL production.

All strains were based on the platform strain “MDO” (e.g., see example 1 and also reported in WO2020255054A1 or WO2019123324A1) and contained the α-1,2-fucolyltransferase from Helicobacter pylori, futC (GenBank assession nr. CP003904), incorporated in a single copy at two different loci in the genome of the MDO strain. The genotypes of the tested strains are given in table 6.

TABLE 6
Genotypes of the 2′FL producing strains
with different expression levels of lacY

| Strain ID | Genotype | Transporter |
|---|---|---|
| MP18 | MDO x2 PglpF-futC, PglpF-nec, 1xCA* | nec |
| MP19 | MDO x2 PglpF-futC, PglpF-nec, | nec |
| | PglpF_SD7-lacY, 1xCA* | |
| MP20 | MDO x2 PglpF-futC, PglpF-nec, | nec |
| | PglpF-lacY, 1xCA* | |

*CA: extra colanic acid gene cluster (gmd-wcaG-wcaH-wcal-manC-manB) under the control of a PglpF promoter at a locus that is different than the native locus.

### Deep-Well Assay

The strains disclosed in the present example were screened for 2′FL production in a 96-deep well plate assay. Three replicates per strain were tested. More specifically, during day 1, fresh precultures were prepared using a basal minimal medium (BMM) supplemented with magnesium sulphate, thiamine and glucose. The precultures were incubated at 34° C. with 700 rpm for 24 h and then transferred to a new BMM (pH 7.5) in order to start the main culture. The new BMM was supplemented with magnesium sulphate, thiamine, a bolus of 20% glucose solution (0.5 L/mL) and a bolus of 10% lactose solution (0.1 μL/μL).

To the main culture 50% (52.5 μl/ml) was provided to the cells accompanied by the addition of sucrose hydrolase (invertase 0.1 g/L), so that glucose was released at a rate suitable for C-limited growth allowing for gene expression and 2′FL production. The main cultures were incubated at 28° C. with 700 rpm for 48 h. For the analysis of total broth, the 96 well plates were boiled at 100° C., subsequently centrifuged, and finally the supernatants were analysed by HPLC.

### Results

The strains of the present example produce 2′FL and very low levels of DFL. The results from deep-well assays are shown in FIG. 9. The 2′FL production for the strain expressing the MFS transporter nec was set to 100 and the corresponding strains with one additional copy of lacY on the chromosome expressed from either PglpF or PglpF_SD7 promoter was normalized to the respective nec transporter strain.

From FIG. 9 it can be seen that in a 2′FL producing strain, overexpression of lacY does not increase the total 2′FL production. In fact, the production seems to be reduced with increased overexpression of lacY as indicated by the strains expressing lacY from a weak promoter (PglpF_SD7 in strain MP19) and from a strong promoter, (PglpF in strain MP20).

1. A genetically engineered cell capable of producing LNT, LNnT, or LNT and LNnT, and wherein the cell
a) overexpresses one or more lactose permease genes,
b) expresses a heterologous MFS transporter protein YberC or a functional homologue thereof having an amino acid sequence which is 95% identical to the amino acid sequence of SEQ ID NO: 67,
c) expresses two or more glycosyltransferases selected from the group consisting of β-1,3-GlcNAc-transferases, β-1,3-Gal-transferases and β-1,4-gal-transferases, and
d) expresses one or more polypeptides involved in the biosynthesis of activated sugars.

2. The engineered cell according to claim 1, wherein the cell further expresses a sucrose utilisation system.

3. The engineered cell according to claim 1, wherein the genetically engineered cell is Escherichia coli.

4. The engineered cell according to claim 1, wherein the cell comprises,
i) one or more lactose permeases selected from the group consisting of SEQ ID NO: 1, 2, 3, 4, 5, 6, 7, 8, 9, 10, 11, 12, 13, 14, 15, and 16; or a functional homologue thereof, having an amino acid sequence which is at least 95% identical to any one of SEQ ID NO: 1, 2, 3, 4, 5, 6, 7, 8, 9, 10, 11, 12, 13, 14, 15, or 16,
ii) two or more glycosyltransferases selected from the group consisting of CvB3galT, GalTK, GalT, LgtA, PmnagT, HD0466, and a functional homologue thereof, having an amino acid sequence which is at least 95% identical to any one of SEQ ID NO: 17 (CvB3galT), SEQ ID NO: 19 (GalTK), SEQ ID NO: 19 (GalT), SEQ ID NO: 20 (LgtA), SEQ ID NO: 21 (PmnagT), or SEQ ID NO: 22 (HD0466), and
iii) one or more polypeptides involved in the biosynthesis of activated sugars selected from the group consisting of SEQ ID NO: 43 or 44 (Pgm), SEQ ID NO: 45 or 46 (GalU), SEQ ID NO: 47 or 48 (GalE), SEQ ID NO: 49 or 50 (GlmM), SEQ ID NO: 51 or 52 (GlmU), and SEQ ID NO: 53 or 541 (GlmS).

5. A method for producing one or more human milk oligosaccharides (HMOs) comprising:
a) providing a genetically engineered cell of claim 1,
b) culturing the cell in a suitable media with added lactose, and
c) harvesting the one or more HMOs,
wherein the one or more HMOs are LNT, LNnT, or both.

6. The method according to claim 4, wherein the genetically engineered cell is of claim 2.

7. The method according to claim 6, wherein the sucrose utilization system comprises a polypeptide capable of hydrolysing sucrose into glucose and fructose, selected from the group consisting of SEQ ID NOs: 86 and 87, or a functional homologue of any one of SEQ ID NOs: 86 and 87, having an amino acid sequence which is at least 95% identical to any one of SEQ ID NOs: 86 or 87.

8. The method according to claim 5, wherein the amino acid sequence of the one or more lactose permeases is selected from the group consisting of SEQ ID NO: 1, 2, 3, 4, 5, 6, 7, 8, 9, 10, 11, 12, 13, 14, 15, and 16, or an amino acid sequence which is at least 95% identical to SEQ ID NO: 1, 2, 3, 4, 5, 6, 7, 8, 9, 10, 11, 12, 13, 14, 15, and 16 and which encodes a functional homologue.

9. The method according to claim 5, wherein
the β-1,3-Gal-transferase is selected from the group consisting of CvB3galT and GalTK, or a functional homologue of CvB3galT or GalTK, having an amino acid sequence which is at least 95% identical to any one of the amino acid sequences of SEQ ID NO: 17 (CvB3galT), SEQ ID NO: 18 (GalTK), or
the β-1,4-gal-transferase is GalT, or a functional homologue of GalT, having an amino acid sequence which is at least 95% identical to SEQ ID NO: 19 (GalT).

10. The method according to claim 5, wherein the β-1,3-GlcNAc-transferase is selected from the group consisting of LgtA, PmnagT, HD0466 and a functional homologue of any one of LgtA, PmnagT or HD0466, having an amino acid sequence which is at least 95% identical to any one of SEQ ID NO: 20 (LgtA), SEQ ID NO: 21 (PmnagT), or SEQ ID NO: 22 (HD0466).

11. The method according to claim 5, wherein the genetically engineered cell further comprises one or more heterologous nucleic acid sequence encoding one or more heterologous polypeptides, which enables utilization of sucrose as sole carbon and energy source of said genetically engineered cell.

12. The method according to claim 5, wherein the genetically engineered cell expresses one or more polypeptides involved in the biosynthesis of activated sugar nucleotides selected from the group consisting of Pgm, GalU, GalE, GlmM, GlmU and GlmS, or a functional homologue thereof having an amino acid sequence which is at least 95% identical to any one of SEQ ID NO: 43 or 44 (Pgm), SEQ ID NO: 45 or 46(GalU), SEQ ID NO: 47 or 48(GalE), SEQ ID NO: 49 or 50(GlmM), SEQ ID NO: 51 or 52 (GlmU), SEQ ID NO: 53 or 54.

13. The method according to claim 5, wherein the genetically engineered cell comprises more than one nucleic acid sequence encoding one or more lactose permeases.

14. The method according to claim 13, wherein at least one of the nucleic acid sequences encoding lactose permease is regulated by one or more promoter sequences selected from the group consisting of Plac, PgatY 70_UTR, PglpF, PglpF_SD1, PglpF_SD10, PglpF_SD2, PglpF_SD3, PglpF_SD4, PglpF_SD5, PglpF_SD6, PglpF_SD7, PglpF_SD8, PglpF_SD9, Plac 16UTR, PmglB 70_UTR, PmglB 70_UTR_SD4, CP6 and PosmY.

15. The method according to claim 5, wherein at least one nucleic acid sequence encoding the one or more lactose permeases is integrated into the genome of the genetically engineered cell.

16. The method according to claim 5, wherein the genetically engineered cell comprises at least one nucleic acid sequence encoding one or more heterologous polypeptides involved in the biosynthesis of activated sugars.

17. The method according to claim 16, wherein the at least one nucleic acid sequence encoding one or more heterologous polypeptides involved in the biosynthesis of activated sugars is integrated into the genome of the genetically engineered cell.

18. The method according to claim 5, wherein at least one nucleic acid sequence encoding the heterologous MFS transporter is integrated into the genome of the genetically engineered cell.

19. The method according to claim 5, wherein the genetically engineered cell is Escherichia coli.

20. The method according to claim 5, wherein the genetically engineered cell is Escherichia coli K-12.
